(19) United States
(12) Patent Application Publication
(10) Pub. No.: US 2004/0072319 A1
(43) Pub. Date: Apr. 15, 2004

(54) Molecules that modulate ubiquintin-dependent proteolysis and methods for identifying same

(76) Inventors: Piers Nash, Ontario (CA); Anthony Pawson, Ontario (CA); Xiaojing Tang, Ontario (CA); Michael Tyers, Ontario (CA)

Correspondence Address: Ropes & Gray, One International Place, Boston, MA, 02110-2624, US

(21) Appl. No.: 10/275,427

(22) PCT Filed: May 4, 2001
(86) PCT No.: PCT/CA01/00632

(51) Int. Cl. C12N 9/64; C12N 15/00

(52) U.S. Cl. 435/226; 435/320.1; 435/325

(57) ABSTRACT

The invention relates to methods for identifying compounds that modulate ubiquitin-dependent proteolysis, and compounds identified using the methods. The invention also relates to a novel peptide motif referred to as the “CPD motif”, molecules derived from the CPD motif, and uses of the CPD motif and molecules.

### FIELD OF THE INVENTION

The invention relates to methods for identifying compounds that modulate ubiquitin-dependent proteolysis, and compounds identified using the methods. The invention also relates to a novel sequence motif referred to as the “CPD motif”, molecules derived from the CPD motif, and uses of the CPD motif and molecules.

### BACKGROUND OF THE INVENTION

Numerous regulatory proteins are degraded in a precisely programmed manner by the ubiquitin system, in which the small protein ubiquitin is covalently conjugated to substrate proteins by the action of an enzymatic cascade, E1->E2->E31. E3 enzymes catalyze the terminal step of ubiquitin transfer to substrates, and as such are the crucial determinants of substrate specificity. Substrate recognition depends on often ill-defined sequence elements, referred to as degrons, that are the binding sites for cognate E3 enzymes (1,2). The E3-substrate interaction can be regulated at several levels. In some instances, limiting cofactors determine E3 activity, as in the case of the Anaphase Promoting Complex/Cyclosome (APC/C), the multisubunit E3 that targets mitotic cyclins and other proteins for degradation during mitosis (3). In other cases, E3 recognition depends on the regulated formation of specific epitopes on substrates. In particular, phosphorylation is often used to direct regulatory proteins to a recently described class of E3 enzymes termed Skp1-Cdc53/cullin-F-box protein (SCF) complexes (4,5). SCF complexes target a broad spectrum of substrates via a large repertoire of substrate-specific adapter subunits called F-box proteins (6). The 40 amino acid F-box motif is a binding site for Skp1, which in turn links F-box proteins to a core ubiquitination complex composed of the scaffold protein Cdc53/Cull, the R[NG-H2 domain protein Rbx1 (a.k.a. Roc1 or Hrt1) and, usually, the E2 enzyme Cdc34 (5). F-box proteins capture phosphorylated substrates via C-terminal protein-protein interaction regions, such as WD40 repeat domains or leucine rich repeat (LRR) domains (7). Phosphorylation-dependent recognition by SCF complexes thus connects kinase-based signalling networks to the ubiquitin system. The mechanisms that account for specific binding of phosphoprotein substrates by F-box proteins are largely unexplored, but might be expected to depend on superposition of recognition sites for the cognate kinases and F-box proteins.

Cell cycle progression depends on the precisely ordered elimination of cyclins and cyclin-dependent kinase (CDK) inhibitors by the ubiquitin system (5,8). In yeast, commitment to division, an event called Start, requires a threshold level of G1 cyclins, which serve to activate Cdc28 (a.k.a. Cdk1) in late G1 phase. As cells pass Start, B-type cyclin Clb-Cdc28 is activated, a necessary step for initiation of DNA replication (9). The primary function of Cln-Cdc28 activity is to phosphorylate an inhibitor of the Clb-Cdc28 kinases called Sic1, thereby targeting it for degradation (9-11). Phospho-Sic1 is specifically recognized by the F-box protein Cdc4, which recruits Sic1 for ubiquitination by the Cdc34-SCF complex (6,7,12). The importance of timely Sic1 degradation is illustrated by the fact that stable forms of Sic1 lacking Cdc28 phosphorylation sites cause a G1 phase arrest (13), whereas deletion of SIC1 causes premature DNA replication and rampant genome instability (14). Cdc4 recruits several other substrates to the SCF core complex in a phosphorylation dependent manner, including the Cln-Cdc28 inhibitor/cytoskeletal scaffold protein Far1, the replication protein Cdc6 and the transcription factor Gcn4 (4).

SCF pathways play analogous roles in the mammalian cell cycle. The LRR-containing F-box protein Skp2 recruits phosphorylated forms of the CDK inhibitor p₂₇^{Kip1} and probably cyclin E, a G1 cyclin, to an SCF complex based on the Cdc53 ortholog Cull (15,16). The crucial role of SCF pathways in mammalian cell division is illustrated by the G1 phase arrest conferred by non-phosphorylatable forms of p27 (17), and by the genome instability caused by expression of stabilized forms of cyclin E (18). SCF-dependent proteolysis also regulates numerous signaling pathways. Most notably, the WD40 repeat containing F-box protein β-TrCP recruits the NFκB inhibitor 1κBα, as well as the growth-regulated transcription factor β-catenin (19). Substrate recognition by β-TrCP depends on phosphorylation of two closely spaced serine residues within a consensus sequence present in both 1κBα and β-catenin. Mutation of these sites renders substrates resistant to degradation, and in the case of β-catenin, the stabilized protein has potent transformation activity. As hundreds of F-box proteins are extant in sequence databases, it seems likely that many cellular pathways will prove to be controlled by SCF mediated protein degradation (4-6).

In spite of the well documented requirement for substrate level phosphorylation in SCF-dependent ubiquitination, the mechanism by which phosphorylation drives substrate binding is not well understood.

### SUMMARY OF THE INVENTION

The SCFcdc4 complex has been implicated in the targeted phosphorylation-dependent ubiquitination of key cell cycle regulatory proteins Sic1, Gcn4, Far1 and Ash1. Cdc4 binds to, and allows SCF-mediated ubiquitination of, mammalian cyclin E1 phosphoprotein. Applicants found that this binding is competed by a cyclin E phosphopeptide corresponding to the region around Thr₃₈₀. This peptide binds to Cdc4 with a K_{D} of 0.8 μM, and is specific for pThr, providing evidence of WD40 domain phosphopeptide recognition. This represents the binding site for multiple Cdc4 target proteins as cyclin E1 peptide competes for the interaction between Cdc4 and its substrates (e.g. Sic1, Far1 and Ash1). Examination of the key determinants of binding in this peptide revealed a conserved phosphorylation-dependent degradation consensus, which is referred to as the “Cdc4 Phospho-Degron motif”, “CPD motif”, or “CPD-box” (the motif is also referred to herein as a “Phosphorylation-Dependent Degradation Signal Box” or “PD-box”). The present inventors demonstrated that the addition of a CPD motif was sufficient to target mutant Sic1 for ubiquitination in vitro and degradation in vivo. The CPD motif exists in the sequence of Gcn4 and Pc17, and acts to target these proteins for ubiquitination by SCFCdc4. Moreover, this is the first demonstration that a small molecule can disrupt interaction of Cdc4 with substrates for ubiquitination.

In carrying out their investigations Applicants generally found that stable binding of F-box proteins to their substrates is achieved through recognition of multiple low affinity phosphoprotein binding sites. This finding has enabled Applicants to develop a method for identifying modulators of ubiquitination of key regulatory proteins. The method involves identifying an amino acid sequence motif on a substrate of an F-box protein that interacts with low affinity with the F-box protein; and optimizing the motif so that it interacts with the F-box protein with high affinity. Such optimized motifs interact with high affinity with the F-box protein and compete with the substrate for binding to the F-box protein. The optimized motifs or peptides derived from the motifs may be used to disrupt degradation of regulatory proteins. Accordingly, they can be used as therapeutic agents to treat cell cycle diseases and other diseases or conditions, for example cancers in which a regulatory protein is being prematurely degraded as a result of an overabundance of its F-box protein binding partner.

Therefore, the present invention provides a method for identifying agents to be tested for their ability to modulate ubiquitin-dependent proteolysis of a regulatory protein, involving interaction of multiple low affinity binding sites on the protein with an F-box protein comprising:

(a) selecting a sequence motif of a low affinity binding site;

(b) optimizing the sequence motif so that a peptide comprising the sequence motif or a peptide mimetic thereof is capable of interacting with the F-box protein with high affinity; and

(c) synthesizing an agent comprising or consisting essentially of a peptide comprising the optimized motif, or peptide mimetic thereof;

(d) optionally testing the peptide or mimetic thereof to ascertain if the peptide or peptide mimetic modulates ubiquitin-dependent proteolysis of the protein, preferably testing the activity of the peptide or peptide mimetic in cellular assays and animal model assays.

Another aspect of the invention provides a peptide or peptidomimetic, e.g., wherein one or more backbone bonds is replaced or one or more sidechains of a naturally occurring amino acid are replaced with sterically and/or electronically similar functional groups.

In other embodiments, the invention provides a method for identifying inhibitors of the F-box interaction, comprising

(a) providing a reaction mixture including the regulatory protein and an F-box protein, or at least a portion of each which interact;

(b) contacting the reaction mixture with one or more test compounds;

(c) identifying compounds which inhibit the interaction of the regulatory and F-box proteins.

In certain preferred embodiments, the reaction mixture is a whole cell. In other embodiments, the reaction mixture is a cell lysate or purified protein composition. The subject method can be carried out using libraries of test compounds. Such agents can be proteins, peptides, nucleic acids, carbohydrates, small organic molecules, and natural product extract libraries, such as isolated from animals, plants, fungus and/or microbes.

Still another aspect of the present invention provides a method of conducting a drug discovery business comprising:

(a) providing one or more assay systems for identifying agents by their ability to inhibit or potentiate the interaction of a regulatory protein and an F-box protein;

(b) conducting therapeutic profiling of agents identified in step (a), or further analogs thereof, for efficacy and toxicity in animals; and

(c) formulating a pharmaceutical preparation including one or more agents identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

Yet another aspect of the invention provides a method of conducting a target discovery business comprising:

(a) providing one or more assay systems for identifying agents by their ability to inhibit or potentiate the interaction of a regulatory protein and an F-box protein;

(b) (optionally) conducting therapeutic profiling of agents identified in step (a) for efficacy and toxicity in animals; and

(c) licensing, to a third party, the rights for further drug development and/or sales for agents identified in step (a), or analogs thereof.

In one embodiment of the subject assay, the target regulatory protein is the tumor suppressor p53, and the assay is used to identify inhibitors of ubiquitin-mediated destruction of p53. Many lines of evidence point to the importance of p53 in human carcinogenesis. For instance, mutations within the p53 gene are the most frequent genetic aberration thus far associated with human cancer. Although p53 can block the progression of the cell cycle when artificially expressed at high levels, it appears to be dispensable for normal development. Thus, for mice containing homozygous deletions and humans harboring germline mutations of p53, development is normal and p53 protein is expressed at very low levels in most cell types. Emerging evidence, however, suggests that p53 is a checkpoint protein that plays an important role in sensing DNA damage or regulating cellular response to stress. Under normal conditions, p53 is an unstable protein and is present at very low levels in the cell, and the level of p53 in a cell appears to be controlled at least in party by degradation involving the ubiquitin system. Treating cells with UV light or X rays dramatically reduces the rate of p53 degradation, leading to a rapid increase in its concentration in the cell and presumably inducing the transcription of genes that block passage through the restriction point. However, while normal cell lines irradiated in G1 fail to enter S phase, many tumor lines do not. In fact, there is a perfect correlation between cell lines that lack this feedback control and cells that have mutations in the p53 gene. These mutations are of two sorts: recessive mutations that inactivate the gene, and dominant mutations that produce abnormal proteins. An inhibitor developed using the subject assay could be used therapeutically to enhance the function of the p53 checkpoint by increasing the steady state concentration of p53 in the treated cell. The anti-proliferative activity of such an inhibitor can be employed in the treatment of hyperplasias or neoplasias by increasing the fortitude of the checkpoint in transformed cells which contain wild-type p53 (i.e. can induce apoptosis in cells overexpressing c-myc), or by offsetting a diminishment in p53 activity by increasing the level of (mutant) p53. Moreover, such agents can also be used prophylactically to increase p53 levels and thereby enhance the protection against DNA damaging agents when it is known that exposere to damaging agents, such as radiation, is imminent.

In other embodiments, the targeted regulatory protein is the p27^{kip1} protein. The CDK complex activity is regulated by mechanisms such as stimulatory or inhibitory phosphorylations as well as the synthesis and degradation of the kinase and cyclin subunit themselves. Recently, a link has been established between the regulation of the activity of cyclin-dependent kinases and cancer by the discovery of a group of CDK inhibitors including the p27^{kip1} protein. The inhibitory activity of p27^{kip1} is induced by the negative growth factor TGF-β and by contact inhibition (Polyak et al., Cell 78:66-69, 1994). These proteins, when bound to CDK complexes, inhibit their kinase activity, thereby inhibiting progression through the cell cycle. Loss of p27^{kip1} protein, e.g., by ubiquitin-mediated degradation, is a prognostic indicator for aggressiveness of certain tumors.

In still other embodiments, the targeted regulatory protein is the IkB protein. NF-κB is a member of the Rel family of proteins; it binds to specific DNA sequences (kB sites) and functions as a transcriptional activator in the nucleus. IkB-α forms a complex with NF-kB that is maintained in the cytoplasm. When NF-kB is activated (for example, in response to cytokines, cellular stress, and reactive oxygen intermediates), IkB's becomes phosphorylated and undergo ubiqutination (Adcock et al. (1994) Biochem. Biophys. Res. Commun. 199:1518; Miyamoto et al. (1994) PNAS 91:12740). The unbound NF-kB then translocates to the nucleus, where it activates transcription.

In another embodiment, the targeted regulatory protein is the myc oncoprotein. The myc regulatory protein is activated by translocation or mutation in many B-cell lymphomas or by amplification in tumor types, such as small cell lung cancer and breast cancer. The c-myc gene is the cellular homolog of the viral oncogene v-myc, which is found in a number of avian and feline retroviruses which induce leukemia and carcinomas. Myc has been implicated in the control of normal cell proliferation by many studies. In particular, it is one of the immediate early growth response genes that are rapidly induced in quiescent cells upon mitogenic induction, suggesting that it plays some role in mediating the transition from quiescence to proliferation. However, increased levels of myc itself is not sufficient to cause proliferation. In fact, in normal cells the opposite happens and the cell undergoes apoptosis. Therefore, inhibitors identified in the present assay can be used to effectively induce apoptosis in cells which do not normally overexpress myc. For example, specific delivery of these agents to lymphocytes can be used to inhibit proliferation of B- and/or T-cells in order to induce clonal deletion and generate tolerance to particular antigens.

In tumor cells, on the other hand, elevated or deregulated expression of c-myc is so widespread as to suggest a critical role for myc gene activation in multi-stage carcinomas (Field et all. (1990) Anticancer Res 10:1-22; and Spencer et al. (1991) Adv Cancer Res 56:1-48). However, such overexpression of myc in these cells is typically believed to be accompanied by expression of other cellular proteins, such as bcl-2. Interestingly, however, almost all tumor cells tested that overexpress myc readily undergo apoptosis in the presence of cytotoxic and growth-inhibitory drugs (Cotter et al. (1990) Anticancer Res 10: 1153-1159; and Lennon et al. (1990) Biochem Soc Trans 18:343-345). Therefore, inhibitors of the ubiquitin-mediated degradation of myc can be used to further deregulate the expression of myc in order to render the cells even more sensitive to a chemotherapeutic treatment, or to possibly upset the careful balance of the transformed cell and cause apoptosis to occur even in the absence of a second cytotoxic drug.

Cyclin degradation is a key step governing exit from mitosis and progression into the next cell-cycle. For example, the transition from metaphase to anaphase which marks the end of mitosis in induced by the degradation of cyclin by a ubiquitin-mediated pathway, which in turn leads to the inactivation of cyclin-dependent kinases (cdk) operational at that cycle-cycle stage. As cells enter interphase, cyclin degradation ceases, cyclin accumulates and, as a result of a complex series of post-translational modifications, cyclin/cdk complexes are activated as kinases which drive the cell through mutosis. Cyclin degradation is thus one of the crucial events in exiting mitosis. Indeed, cyclin mutants that retain the ability to activate the cdk complexes, but which cannot be degraded, arrest the cell-cycle in mitosis. Similar cyclin-dependence exists at other points of the cell-cycle as well. Thus, inhibitors of ubiquitin-mediated degradation of a cyclin (such as where the cyclin is chosen from cyclin A, B, C, D1, D2, D3, E or F) can be used as antiproliterative agents. In one aspect of the invention, an inhibitor of ubiquitin-mediated cyclin degradation can be generated for use as fungal antiproliterative agents. For instance, genetic screens have identified three yeast cyclins, CLN1, CLN2, and CLN3, in S. cerevisiae that cooperate with cdc28 at start. The cdc34 gene has been identiified in S. cerevisiae to encode a ubiquitin-conjugating enzyme which involved in ubiquitination of CLN3. Inhibitors of cdc34 identifed in the present invention can therefore be of potential use in treating, for example, mycotic infections.

The fos oncogene product, which can undergo ubiquitin-mediated degradation in a cell, has been implicated in neoplastic transformation as well as in mediating the action of a variety of extracellular stimuli. The control of gene expression by c-fos is believed to play a critical role in cellular proliferation and developmental responses, and alterations in the normal pattern of c-fos can lead to oncogenesis. Given the prominence of c-fos as an early response gone, apparent over-expression and prolonged lifetime of c-fos, as may be caused by an inhibitor of the ubiquitin-mediated degradation of c-fos, might sufficiently unbalance the cell-cycle and cause cell death. Alternatively, such inhibitors can be used to mimic the effects of an external stimulus on the cell, such as treatment with a cytokine.

Another regulatory protein that is short-lived due to ubiquitin-mediated degradation is for the yeast MATα2 transcriptional regulator of S. cervesiae, which governs the cell identity between the haploid forms, a and α, and the a/α diploid. Mutants deficient in the degradation of MATα2 have been found to have a number of defects, including inhibition of growth (Hochstrasser et al. (1990). Cell 61:697-708; and Chen et al. (1993) Cell 74: 357-369). Thus, the subject method can be used to identify inhibitors of ubiquitin-mediated degradation of MATα2. Such inhibitors can be useful in, for example, the treatment of mycotic infections, as well as the preservation of foodstuff.

The method may further comprise the steps of preparing a quantity of the agent and/or preparing a pharmaceutical composition comprising the agent.

The invention also contemplates the agents (e.g. motifs, peptides comprising the motifs, and peptide mimetics thereof) identified using this method of the invention. The agents may be used to disrupt ubiquitin-dependent proteolysis of a regulatory protein (ie. stabilize a regulatory protein), or they may be used to selectively degrade a target protein. In certain preferred embodiments, the subject method can be used to identify ubiquitination inhibitors having molecular weights less than 5000 amu, more preferably less than 2500 amu, and most preferably less than 1000 amu, e.g, to identify small organic molecule inhibitors. In an embodiment of the invention a CPD motif that targets molecules for ubiquitin-dependent proteolysis is provided. Preferably, the CPD motif is an isolated CPD motif. A “CPD motif” may comprise the consensus sequence X²-X³-pThr-Pro-X⁴, more particularly X²-X³-pThr-Pro-X⁴-X⁵-X⁶-X⁷ where X² to X⁷ inclusive are as described herein. A CPD motif may comprise the consensus sequence X¹-Leu/Gly/Tyr-Pro-pThr-Pro-X⁹ where X¹ and X⁹ are as described herein. A CPD motif may be from any species, particularly a mammalian species, including bovine, ovine, porcine, murine, equine, preferably the human species, and from any source, whether natural, synthetic, semi-synthetic, or recombinant. Preferably the CPD motif is a Cyclin E1, Gcn4, Far1, Ash1, Sic1, Cdc16, or Pc17 CPD motif. The term “CPD motif” also includes polypeptides that are homologous to a CPD motif.

The present invention also relates to molecules derived from a CPD motif, or a CPD motif binding partner.

In an embodiment, the invention relates to a molecule derived or optimized from a CPD motif of cyclin E. In particular, the invention provides a CPD peptide of the formula:

X¹-X²-X³-pThr-Pro-X⁴-X⁸

wherein X¹ represents 0 to 100 amino acids, preferably 0 to 50, more preferably 0 to 20, most preferably 0 to 10 amino acids, X² represents Leu, Pro, or Ile, preferably Leu or Ile; X³ represents Leu, Ile, Val, or Pro, preferably Ile, Leu, or Pro; X⁴ represents any amino acid except basic and bulky hydrophobic amino acids, preferably X⁴ is any amino acid except Arg, Lys, or Tyr more preferably X4 is Ile, Val, Pro, or Gln, and X⁸ represents 0 to 100 amino acids, preferably 0 to 50, more preferably 0 to 20, most preferably 0 to 10 amino acids.

In another embodiment of the invention a CPD peptide is provided of the formula:

X¹-X²-X³-pThr-Pro-X⁴-X⁵-X⁶-X⁷-X⁸

wherein X¹ represents 0 to 100 amino acids, preferably 0 to 50, more preferably 0 to 20, most preferably 0 to 10 amino acids; X² represents Leu, Pro, or Ile, preferably Leu or Ile; X³ represents Leu, Ile, Val, or Pro, preferably Ile, Leu, or Pro; X⁴, X⁵ and X⁶ represent any amino acid except basic and bulky hydrophobic amino acids, preferably X⁴ is any amino acid except Arg, Lys, Tyr, or Trp, more preferably X⁴ is Ile, Val, Pro, or Gln, preferably X⁵ and X⁶ are any amino acid except Arg, Lys, or Tyr and more preferably X⁵ is Gln, Leu, Met, Thr, or Glu, and X⁶ is Gln, Ala, Thr, Glu, or Ser; X⁷ is any amino acid, preferably not a basic or bulky hydrophobic amino acid, more preferably X⁷ is any amino acid except Arg, Lys, or Tyr, most preferably X⁷ is Leu, Trp, Asp, Pro, or Gly; and Xs represents 0 to 100 amino acids, preferably 0 to 50, more preferably 0 to 20, most preferably 0 to 10 amino acids.

A CPD peptide or peptide mimetic of the invention preferably binds to a CPD motif binding partner (e.g. Cdc4) with a K_{d} of less than 25 μM, and more preferably less than 1 μM, 100 nM or even 10 nM, and is capable of disrupting or promoting the interaction of a CPD motif and a CPD motif binding partner, or mediating ubiquitin-dependent proteolysis.

The invention also encompasses molecules derived from a CPD peptide of the invention.

The molecules and CPD peptides of the invention may disrupt or promote the interaction of a CPD motif and a CPD motif binding partner. In a preferred embodiment, the molecules or CPD peptides bind to, or alter the function of an SCF complex, preferably a mammalian SCF complex.

The invention also relates to novel chimeric proteins, and DNA constructs encoding them. The chimeric proteins contain at least one CPD motif or molecule derived from a CPD motif (e.g. a peptide of the invention) fused to a target protein and/or a targeting domain capable of directing the chimeric protein to a desired cellular component or specific cell type or tissue. The chimeric proteins may also contain additional amino acid sequences or domains.

The invention contemplates a complex comprising a CPD motif and a substance that binds to a CPD motif (i.e. CPD motif binding partner) including an F-box Protein.

The invention also provides nucleic acid molecules that encode a CPD motif, CPD peptide, CPD binding partner, or chimeric protein of the invention. These molecules may be used for the genetic engineering of host cells in vivo or in vitro. Also provided are methods and compositions for producing and using the modified cells. In an embodiment of the invention, DNA vectors are contemplated containing a nucleic acid molecule of the invention whether for introduction of the nucleic acid molecule into host cells in vitro or for administration to whole organisms for introduction into cells in vivo. Accordingly, vectors may be constructed which comprise a nucleic acid molecule of the invention, and where appropriate one or more transcription and translation elements linked to the nucleic acid molecule.

A CPD motif, CPD peptide, CPD binding partner, or chimeric protein of the invention can be produced by recombinant procedures. In one aspect the invention provides a method for preparing a CPD motif, CPD peptide, CPD binding partner, or chimeric protein of the invention utilizing an isolated nucleic acid molecule of the invention. In an embodiment, a method for preparing a CPD motif, CPD peptide, CPD binding partner, or chimeric protein of the invention is provided comprising: (a) transferring a vector of the invention into a host cell; (b) selecting transformed host cells from untransformed host cells; (c) culturing a selected transformed host cell under conditions which allow expression of the CPD motif, CPD peptide, CPD binding partner, or chimeric protein, and (d) isolating the CPD motif, CPD peptide, CPD binding partner, or chimeric protein. The invention further broadly contemplates a recombinant molecule obtained using a method of the invention.

Still further the invention provides an antibody specific for a CPD motif, CPD peptide, CPD binding partner, chimeric protein, or nucleic acid molecule of the invention. Antibodies may be labeled with a detectable substance and used to detect proteins or complexes of the invention in biological samples, tissues, and cells. Antibodies may have particular uses in therapeutic applications, and in conjugates and immunotoxins as target selective carriers of various agents which have therapeutic effects including chemotherapeutic drugs, toxins, immunological response modifiers, enzymes, and radioisotopes.

In accordance with an aspect of the invention there is provided a method of, and products for, diagnosing and monitoring conditions characterized by an abonormality in a signal transduction pathway involving the interaction of a CPD motif and a CPD motif binding partner comprising determining the presence of (a) a nucleic acid molecule encoding a CPD motif or CPD binding partner (b) a CPD motif or CPD motif binding partner, or (c) complexes of the invention.

The invention still further provides a method for identifying a substance which interacts with or binds to a CPD motif, CPD motif containing protein, or a molecule derived from a CPD motif (e.g. CPD peptide) comprising (a) reacting the CPD motif, protein, or molecule with at least one substance which potentially can interact with or bind to the CPD motif, protein, or molecule (i.e. CPD motif binding partner) under conditions which permit the formation of complexes between the substance and CPD motif, protein, or molecule, and (b) detecting binding, wherein detection of binding indicates the substance binds to the CPD motif, protein, or molecule. Binding can be detected by assaying for complexes, for free substance, for non-complexed CPD motif, protein, or molecule, or for activation of the CPD motif, protein, or molecule (e.g. phosphorylation). The invention also contemplates methods for identifying substances that bind to other intracellular proteins that interact with a CPD motif. The invention also encompasses the substances identified using this method of the invention.

Still further the invention provides a method for evaluating a compound for its ability to modulate ubiquitin-dependent proteolysis through the CPD motif. For example, the compound may be a substance which binds to a CPD motif or a molecule derived from a CPD motif (e.g. CPD peptides), or a substance which disrupts or promotes the interaction of molecules in a complex of the invention. In an embodiment, the method comprises providing a known concentration of a CPD motif, a molecule derived from a CPD motif, or a molecule of a complex of the invention, with a substance which binds to the CPD motif or molecule (e.g CPD motif binding partner), and a test compound under conditions which permit the formation of complexes between the substance and CPD motif or molecule, and removing and/or detecting complexes. A substance which binds to a CPD motif, or a molecule derived from a CPD motif may be an F-box Protein, preferably a WD40-repeat protein. The invention also encompasses the compounds identified using this method of the invention.

The invention also provides a method for identifying an agent to be tested for an ability to modulate a signal transduction pathway by testing for the ability of the agent to affect the interaction between a CPD motif and CPD motif binding partner, wherein a complex formed by such interaction is part of the signal transduction pathway. In an embodiment, the method comprises (a) exposing at least one agent to a CPD motif for a time sufficient time to allow binding of the agent to the CPD motif; (b) removing non-bound agents; and (c) determining the presence of agent bound to CPD motif thereby identifying an agent to be tested for an ability to modulate a signal pathway.

The invention provides for the use of a CPD motif to promote degradation of a target protein in a cell by ubiquitin-dependent proteolysis. The invention also contemplates a method for selectively degrading a target protein in a cell by ubiquitin-dependent proteolysis comprising administering to the cell a CPD motif, or molecule derived from a CPD motif, preferably a CPD peptide of the invention, in an amount effective to selectively degrade the target protein in the cell. The CPD motif or molecule may be introduced or incorporated into the target protein in the cell.

In yet another aspect the invention provides a method of treating diseases or conditions where the affected cells have a defective target protein (e.g. mutated target protein or over expressed target protein) comprising administering an effective amount of a CPD motif to promote degradation of the target protein in the cell by ubiquitin-dependent proteolysis. To produce modified cells a nucleic acid molecule of the invention is introduced into selected host cells. This may be accomplished using conventional vectors (various examples of which are commercially available) and techniques.

Still further the invention provides for the use of a CPD motif to disrupt degradation of a CPD motif containing protein.

The CPD motif, molecules derived from a CPD motif, CPD peptides, CPD motif binding partners, antibodies, chimeric proteins, agents, substances, and compounds of the invention may be used to modulate ubiquitin dependent proteolysis, and they may be used to modulate cellular processes of cells (such as proliferation, growth, and/or differentiation, in particular glucose and methionine biosynthesis, gene expression, cell division, and transcription) in which the CPD motif, molecules, CPD peptides, CPD motif binding partners, antibodies, chimeric proteins, agents, compounds or substances are introduced.

Accordingly, the CPD motif, molecules derived from a CPD motif, CPD peptides, antibodies, CPD motif binding partners, chimeric proteins, agents, substances, and compounds of the invention may be formulated into compositions for administration to individuals suffering from a proliferative or differentiative condition. Therefore, the present invention also relates to a composition comprising one or more of a CPD motif, molecules derived from a CPD motif, CPD peptides, CPD motif binding partners, antibodies, chimeric proteins, agents, substances, and compounds of the invention, and a pharmaceutically acceptable carrier, excipient or diluent. A method for modulating proliferation, growth, and/or differentiation of cells is also provided comprising introducing into the cells a CPD motif, molecules derived from a CPD motif, CPD peptides, antibodies, chimeric proteins, agents, substances, and compounds of the invention or a composition containing same. Methods for treating proliferative and/or differentiative conditions or diseases using the compositions of the invention are also provided.

Still further the invention provides the use of a CPD motif, molecule derived from a CPD motif, CPD peptides, CPD motif binding partners, antibodies, chimeric proteins, agents, substances and compounds of the invention in the preparation of a medicament to modulate ubiquitin-dependent proteolysis in cells of an individual. The invention also contemplates the use of a CPD motif, molecule derived from a CPD motif, CPD peptides, CPD motif binding partners, antibodies, chimeric proteins, agents, substances and compounds of the invention in the preparation of a medicament to treat individuals suffering from a proliferative or differentiative condition.

The disruption or promotion of the interaction between the molecules in complexes of the invention is useful in therapeutic procedures. Therefore, the invention features a method for treating a subject or individual having a disease or condition characterized by an abnormality in a signal transduction pathway wherein the signal transduction pathway involves an interaction between a CPD motif and a CPD motif binding partner. The condition may also be characterized by an abnormal level of interaction between a CPD motif and a CPD motif binding partner. The method includes disrupting or promoting the interaction (or signal) in vivo. The method also involves inhibiting or promoting the activity of a complex formed between a CPD motif and a CPD motif binding partner.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DESCRIPTION OF THE DRAWINGS

The invention will be better understood with reference to the drawings in which:

FIG. 1 are blots showing (A) the capture of Cdc4 from baculo-lysates using cycE19P, and (B) Ccdc4 ubiquitinates cyclinE; and (C) SCF^{cdc4} ubiquitination of cyclinE in response to CDK phosphorylation of cyclin E.

FIG. 2 shows (A) a Mmplot for cycEpT, Gcn4, cycEpS and cycET peptides and a Hill plot for cycEpT; (B) the deletion constructs of cdc4 tested for Skp1 binding and cycEpT peptide binding; and (C) blots demonstrating that cycEpT peptide inhibits the interaction of Sic1, cyclinE1, and Far1 with Cdc4.

FIG. 3 shows (A) SPOTS blots with cycEpT peptide variations probed with Cdc4/Skp1; (B) CPD-box consensus; and (C) binding kinetics for various CPD-box peptides.

FIG. 4 shows (A) a blot illustrating Cdc4/Skp1 Flag binding to pSic1 mutants and Sic1-CycE chimera; and (B) in vitro ubiquitination of Sic1-CycE chimera.

FIG. 5 shows the contribution of CDK phosphorylation sites to Sic1 recognition, ubiquitination and degradation. a, Consensus S/T-P CDK phosphorylation sites in Sic1 b, Inhibition of Clb5-Cdc28 kinase activity by purified Sic1 phosphorylation site mutants. Histone H1 (HH1) was used as an exogenous substrate to indicate total kinase activity c, Half-life of individual Sic1 phosphorylation site mutants. Decay of Sic1 signal upon repression of the various GAL1-SIC1^{HA} constructs in G1 phase cells was followed by anti-HA immunoblot. The row labelled α-factor indicates signal for wild type Sic1 isolated from a culture maintained in the continuous presence of α-factor to demonstrate Cln-Cdc28 dependence of Sic1 degradation d, Binding of individual Sic1 phosphorylation site mutants to Cdc4. Sic1 wild type and mutant proteins purified as Gst fusions were phosphorylated by Cln2-Cdc28 and then captured onto Cdc₄^{FLAG} immobilized on anti-FLAG resin and detected with anti-Sic1 antibody. Note that unphosphorylated Sic1 co-migrates with Sic1^{9m} and that the hyperphosphorylated species present in some preparations do not influence binding to Cdc4 e, Re-introduction of up to five phosphorylation sites into Sic1^{9m} fails to restore Cdc4 binding in vitro. Restored sites are indicated as follows: 7m=T45, S76; 6m=T33, T45, S76; 4m=T2, T5, T33, T45, S76 f, Re-introduction of up to five phosphorylation sites into Sic1^{9m} fails to overcome lethality upon overexpression from the GAL1 promoter. Strains indicated as in part e were streaked on glucose or galactose medium and incubated for 2 days at 30° C.

FIG. 6 shows a cyclin E1 derived phosphopeptide defines a single high affinity binding site on Cdc4. a, A phosphopeptide corresponding to residues 371-389 of cyclin E1 (CycE^{19-pT380}) captures recombinant Cdc4 from insect cell lysates, whereas a non-phosphorylated CycE^{19-pT380} peptide does not b, Michaelis-Menton plot, Scatchard plot and (inset) Hill plot for the CycE^{19-pT380} phosphopeptide interaction with Skp1-Cdc4 as measured by fluorescence polarization c, Equilibrium binding constants for the Cdc4-CycE^{19-pT380} phosphopeptide interaction determined by fluorescence polarization for a series of Cdc4 deletion mutants. A qualitative assessment of Skp1 binding to Cdc4 determined by anti-Skp1 immunoblot is also indicated d, Phosphorylation-dependent ubiquitination of cyclin E1 by SCF^{Cdc4} in vitro. Recombinant cyclin E1^{MYC6}-Cdk2 complexes purified from transfected COS7 cells were incubated with ATP prior to in vitro ubiquitination. ET^{380A} indicates a mutant cyclin E1 that lacks the T380 phosphorylation site and K2DN indicates a catalytically inactive version of Cdk2 e, Cyclin E1 degradation in yeast depends on Cdc4 function and on phosphorylation of T380. GAL1-cyclin E1 constructs were expressed in the indicated strains by growth in galactose medium then repressed by addition of glucose and cycloheximide, after which cyclin E1 abundance was followed by anti-cyclin E1 immunoblot f, The CycE^{19-pT380} phosphopeptide out-competes binding of Sic1 and cyclin E1 to Cdc4-Skp1 complexes. Increasing concentrations of the indicated peptides (+, 3 μM, ++, 17 μM, +++68 μM) were incubated with Cdc4^{FLAG}-Skp1 resin. Bound proteins were detected with anti-Sic1 and anti-cyclin E1 antibodies.

FIG. 7 shows the delineation of the Cdc4 phosph-degron (CPD) consensus sequence. A membrane bound array of synthetic peptides in which every position in the CycE^{19-pT380} sequence was systematically substituted with every natural amino acid (shown in one letter code), was incubated with purified Skp1-Cdc4 complex followed by detection with an anti-Skp1 antibody.

FIG. 8 shows that the CPD motif is a portable phospho-degron. a, Sic1^{9m} with a CycE^{19-pT380} insert at T45 or the core CPD motif (LLpTPP) substituted at either T45 or S76, are efficiently captured by Cdc4. The indicated purified Gst-Sic1 fusion proteins were either unmodified or phosphorylated with Cln2-Cdc28 and captured on Skp1^{FLAG}-Cdc₄ resin, or as a control Skp1^{FLAG} resin. Inputs shown are 40% of non-phosphorylated and phosphorylated proteins in the binding reaction. T45PSR indicates a mutant in which the T45 site is converted to an optimal CDK phosphorylation site, while S76S and T45T are single wild type sites reintroduced into Sic1^{9m} b, Sic1^{9m} with a CycE^{19-pT380} insert at T45 or the core CPD motif (LLpTPP) substituted at either T45 or S76 Sic1^{9m} are ubiquitinated by recombinant SCF^{Cdc4}. Detection was with anti-Sic1 antibody. c, Introduction of the CycE^{19-pT380} sequence or the CPD core motif into Sic1^{9m} overcome lethality upon overexpression from the GAL1 promoter. Strains bearing a CEN plasmid with indicated GAL1-SIC1 alleles were streaked on glucose or galactose medium and incubated at 300 for 2 days.

FIG. 9 shows premature DNA replication and genome instability caused by introduction of a single optimal CPD motif. a, Strains bearing integrated wild type or SIC1^{7mS76LLpTPP} alleles were synchronized in G1 phase with α-factor and released into fresh raffinose medium at 25° C. Total DNA content was assessed by FACS analysis b, Compromised G1 cyclin activity uncovers premature replication in a SIC1^{9mS76LLpTPP} strain. Asynchronous cultures of SIC1 or SIC1^{7mS76LLpTPP} strains in a cln1 background were grown to mid-log phase in glucose medium at 30° C. and analyzed for total DNA content c, Genome instability caused by the SIC1^{7mS76LLpTPP} allele. Each of the indicated strains carried a marker chromosome that confers an Ade+ phenotype (white colonies); red sectors indicate a chromosome loss event. Representative regions of each streak are shown. Primary chromosome loss events were determined by scoring 4,000 individual colonies for half or greater red sectors d, Synthetic lethal interaction between cdh1Δ and the SIC1^{7mS76LLpTPP} allele. Representative tetrads from a cdh1::HIS3 and sic1::SIC1^{7mS76LLpTPP}-URA₃ and cdh1::HIS3 and sic1::SIC1-URA3 crosses are shown. In the corresponding schematic, H and U indicate deduced His and Ura prototrophy. Of 66 tetrads from the cdh1::HIS3 and sic1::SIC1^{9mS76LLpTPP}-URA₃ cross, 46 His+ Ura+ spore clones did not form colonies, while 19 formed small colonies that could not be propagated. Of 31 tetrads from the cdh1::HIS3 and sic1:: SIC1-URA3cross, two His⁺ Ura⁺ spore clones did not form colonies, while 20 formed normal sized colonies, all of which could be stably propagated.

FIG. 10 shows SPOTS blot optimization of the CPD derived from a Gcn4 peptide. The seed sequence derived from Gcn4 is shown in the left column, whereas systematic single amino acid substitutions made in the Gcn4 sequence are shown in the top row. The optimized CPD consensus closely matches that derived by beginning with the cyclin E T380 peptide, demonstrating the reliability of the optimization method.

FIG. 11 shows a sequence alignment to identify a CPD binding site in Cdc4 and related F-box proteins from other species. Conserved Arg residues demonstrated to be necessary for CPD interaction in vivo and in vitro are circled. Modelling of Cdc4 WD40 repeat domain structure on the known structure of b-transducin demonstrates that the essential Arg residues converge to form a basic binding pocket for the phosphorylated CPD peptides.

FIG. 12 shows conserved surface Arg residues identified by sequence alignment in FIG. 11 are required for Cdc4 function in vivo, as shown by inability of mutant forms to support viability of yeast lacking endogenous Cdc4 (top). Recombinant mutant proteins are unable to support CPD peptide binding in an in vitro fluorescence polarization assay (bottom). Inset shows equal expression and solubility of mutant proteins compared to wild type.

### DETAILED DESCRIPTION OF THE INVENTION

In accordance with the present invention there may be employed conventional molecular biology, microbiology, and recombinant DNA techniques within the skill of the art. Such techniques are explained fully in the literature. See for example, Sambrook, Fritsch, & Maniatis, Molecular Cloning: A Laboratory Manual, Second Edition (1989) Cold Spring Harbor Laboratory Press, Cold Spring Harbor, N.Y); DNA Cloning: A Practical Approach, Volumes 1 and 11 (D. N. Glover ed. 1985); Oligonucleotide Synthesis (M. J. Gait ed. 1984); Nucleic Acid Hybridization B. D. Hames & S. J. Higgins eds. (1985); Transcription and Translation B. D. Hames & S. J. Higgins eds (1984); Animal Cell Culture R. I. Freshney, ed. (1986); Immobilized Cells and enzymes IRL Press, (1986); and B. Perbal, A Practical Guide to Molecular Cloning (1984).

Glossary

Abbreviations for amino acid residues are the standard 3-letter and/or 1-letter codes used in the art to refer to one of the 20 common L-amino acids. Likewise abbreviations for nucleic acids are the standard codes used in the art.

The term “agonist” of a polypeptide of interest, for example, a CPD motif or CPD motif binding partner, refers to a compound that interacts with the polypeptide and maintains or increases the activity of the polypeptide to which it binds. Agonists may include proteins, peptides, nucleic acids, carbohydrates, or any other molecules that bind to a complex of the invention or molecule of the complex, or CPD motif, or CPD motif binding partner. Agonists also include a molecule derived from a motif, preferably a CPD motif, or derived from a CPD motif binding partner. Peptide mimetics, synthetic molecules with physical structures designed to mimic structural features of particular peptides, may serve as agonists. The stimulation may be direct, or indirect, or by a competitive or non-competitive mechanism.

The term “antagonist”, as used herein, of a polypeptide of interest, for example, a CPD motif or CPD motif binding partner, refers to a compound that binds the polypeptide but does not maintain the activity of the polypeptide to which it binds. Antagonists may include proteins, peptides, nucleic acids, carbohydrates, or any other molecules that bind to a complex, or molecule of a complex, a CPD motif, or a CPD motif binding partner. Antagonists also include a molecule derived from a motif, preferably a CPD motif, or derived from a CPD motif binding partner. Peptide mimetics, synthetic molecules with physical structures designed to mimic structural features of particular peptides, may serve as antagonists. The inhibition may be direct, or indirect, or by a competitive or non-competitive mechanism.

“Regulatory protein” refers to a protein that interacts with an F-box protein targeting it for ubiquitin-dependent proteolysis, or a protein targeted for F-box dependent degradation. Examples of regulatory proteins include CPD motif containing proteins including Gcn4, CyclinE, Far1, Ash1, Sic1, Pc17, and Cdc16; p27^{kip1}; Cln2; and, transcription factors such as β catenin or Iκβα.

“CPD motif containing protein” refers to a protein comprising a CPD motif including but not limited to Gcn4, CyclinE, Far1, Ash1, Sic1, Pc17, and Cdc16. Other proteins containing CPD motif sequences may be identified with a protein homology search, for example by searching available databases such as GenBank or SwissProt and various search algorithms and/or programs may be used including FASTA, BLAST (available as a part of the GCG sequence analysis package, University of Wisconsin, Madison, Wis.), or ENTREZ (National Center for Biotechnology Information, National Library of Medicine, National Institutes of Health, Bethesda, Md.).

A “CPD motif binding partner” refers to an amino acid sequence or any other cellular molecule that interacts with or binds a CPD motif. The term includes ligands and/or substrates for the CPD motif as well as CPD motif agonists or antagonists. In a prefered embodiment the interaction is specific i.e. the binding partner does not interact or interacts to a lesser extent with non-CPD motifs. The K_{d} for the interaction between the CPD motif and CPD motif binding partner is preferably less than 25 μM, and more preferably less than 1 μM, 100 nM or even 10 nM. Preferred binding partners are F-box proteins that interact with a CPD motif, preferably amino acid sequences of F-box proteins that interact with a CPD motif.

“F-box Protein” refers to a protein having a characteristic structural motif called the F-box as described in Bai et al, 1996. Examples of F-box Proteins include, pop1/2, Met30, Scon2/Scon3, β-TRCP, MD6, dactylin, cyclin-F, NFB42, WD40-repeat proteins including Cdc4, leucine rich repeat proteins including Grr1 and Skp2, and several other yeast and mammalian proteins (Bai et al, 1996; Cell 86: 263-274, J. Winston et al, Current Biology Vol. 9: 1180-1182, 1999, C. Cenciarelli, et al Current Biology Vol 9: 1177-1179, 1999), and homologs or portions thereof. An F-box Protein also includes a part of the protein preferably a binding domain of the protein that interacts with a CPD or like motif

“WD40-repeat protein” refers to a family of proteins comprising 7 WD40 repeat sequences forming a characteristic propeller-like structure. Examples of WD-repeat proteins are Cdc4. A WD40-repeat protein also includes a part of the protein, preferably a binding domain of the protein that interacts with a CPD motif or like motif.

By being “derived from” a sequence motif (e.g. CPD motif) or binding partner (e.g. CPD motif binding partner) is meant any molecular entity which is identical or substantially equivalent to the motif or binding partner. A peptide derived from a specific binding domain may encompass the amino acid sequence of a naturally occurring motif (e.g. CPD motif), any portion of that motif, or other molecular entity that functions to bind to an associated or interacting molecule (e.g. CPD motif binding partner such as an F-box Protein). A peptide derived from such a motif will interact directly or indirectly with an associated molecule in such a way as to mimic the native motif or binding partner Such peptides may include competitive inhibitors, peptide mimetics, and the like. The entity will not include a full length sequence of a wild-type molecule. Peptide mimetics, synthetic molecules with physical structures designed to mimic structural features of particular peptides, may serve as inhibitors or enhancers.

“Peptide mimetics” or “peptidomimetics” are structures which serve as substitutes for peptides in interactions between molecules (See Morgan et al (1989), Ann. Reports Med. Chem. 24:243-252 for a review). Peptide mimetics include synthetic structures which may or may not contain amino acids and/or peptide bonds but retain the structural and functional features of a peptide, or agonist or antagonist (i.e. enhancer or inhibitor) of the invention. Peptide mimetics also include peptoids, oligopeptoids (Simon et al (1972) Proc. Natl. Acad, Sci USA 89:9367); and peptide libraries containing peptides of a designed length representing all possible sequences of amino acids corresponding to a motif, peptide, or agonist or antagonist (i.e. enhancer or inhibitor) of the invention.

Sequences are “homologous” or considered “homologues” when at least about 70% (preferably at least about 80 to 90%, and most preferably at least 95%) of the nucleotides or amino acids match over a defined length of the molecule. “Substantially homologous” also includes sequences showing identity to the specified sequence. Percent identity can be determined electronically, e.g., by using the MEGALIGN program (DNASTAR, Inc., Madison Wis.) which can create alignments between two or more sequences according to different methods, e.g., the clustal method. (See, e.g., Higgins, D. G. and P. M. Sharp (1988) Gene 73:237-244.) Percent identity can also be determined by other methods known in the art, (e.g., the Jotun Hein method. (See, e.g., Hein, J. (1990) Methods Enzymol. 183:626-645) or by varying hybridization conditions).

Preferably, the amino acid or nucleic acid sequences have an alignment score of greater than 5 (in standard deviation units) using the program ALIGN with the mutation gap matrix and a gap penalty of 6 or greater (Dayhoff).

The terms “interact”, “interaction”, or “interacting” refer to any physical association between proteins, other molecules such as lipids, carbohydrates, nucleotides, and other cell metabolites. Examples of interactions include protein-protein interactions, protein-lipid interactions, and lipid-lipid interactions. The term preferably refers to a stable association between two molecules due to, for example, electrostatic, hydrophobic, ionic and/or hydrogen-bond interactions under physiological conditions. Certain interacting or associated molecules interact only after one or more of them has been stimulated (e.g. phosphorylated). An interaction between proteins and other cellular molecules may be either direct or indirect. An example of an indirect interaction is the independent production, stimulation, or inhibition of both a CPD motif or a CPD motif binding partner by a regulatory agent. Various methods known in the art can be used to measure the level of an interaction. For example, the strength of covalent bonds may be measured in terms of the energy required to break a certain number of bonds.

The term “isolated CPD motif” refers to a CPD motif substantially free of cellular material, or culture medium when produced by recombinant DNA techniques, or chemical reactants, or other chemicals when chemically synthesized. An isolated CPD motif is also preferably free of sequences which naturally flank the motif or domain.

“Ubiquitin-dependent proteolysis” refers to the degradation of proteins by the proteosome or via the endocytic route through ubiquitin conjugation. Ubiquitin conjugation proceeds via a reaction cascade involving ubiquitin-activating (E1), ubiquitin-conjugating (E2) enzymes, and ubiquitin-protein ligases (E3). (See M. Hochstrasser, Annu. Rev. Genet. 1996, 30: 405-39, 1996 for a review of ubiquitin-dependent proteolysis). The term preferably refers to eukaryotic ubiquitin-dependent proteolysis, more preferably mammalian ubiquitin-dependent proteolysis, most preferably human ubiquitin-dependent proteolysis.

“Signal transduction pathway” refers to the sequence of events that involves the transmission of a message from an extracellular protein to the cytoplasm through the cell membrane. Signal transduction pathways contemplated herein include pathways involving a regulatory protein or motif (e.g. CPD motif) or a complex of the invention or an interacting molecule thereof. The amount and intensity of a given signal in a signal transduction pathway can be measured using conventional methods (See Example herein). For example, the concentration and localization of various proteins and complexes in a signal transduction pathway can be measured, conformational changes that are involved in the transmission of a signal may be observed using circular dichroism and fluorescence studies, and various symptoms of a condition associated with an abnormality in the signal transduction pathway may be detected.

“Disease” or “condition” refers to a state that is recognized as abnormal by the medical community. The disease or condition may be characterized by an abnormality in a signal transduction pathway in a cell wherein one of the components of the signal transduction pathway is a regulatory protein or sequence motif thereof, for example a CPD motif containing protein or CPD motif thereof.

“Abnormality” or “abnormal” refers to a level which is statistically different from the level observed in organisms not suffering from a disease or condition. It may be characterized by an excess amount, intensity or duration of signal, or a deficient amount, intensity or duration of signal. An abnormality may be realized in a cell as an abnormality in cell function, viability, or differentiation state. An abnormal interaction level may be greater or less than a normal level and may impair the performance or function of an organism.

Methods for Identifying Agents

The present invention provides a method for identifying agents to be tested for their ability to modulate ubiquitin-dependent proteolysis of a regulatory protein involving interaction of multiple low affinity binding sites on the protein with an F-box protein comprising:

(a) selecting a sequence motif of a low affinity binding site;

(b) optimizing the sequence motif so that a peptide comprising the sequence motif or a mimetic thereof is capable of interacting with the F-box protein with high affinity; and

(c) synthesizing an agent comprising or consisting essentially of a peptide comprising the optimized sequence motif or peptide mimetic thereof;

(d) optionally testing the agent to ascertain if the agent modulates ubiquitin-dependent proteolysis of the protein.

The method involves selecting a sequence motif of a low affinity binding site of a cell cycle regulatory protein. A low affinity binding site interacts with an F-box protein with a K_{d} of greater than 25 μM. The sequence motif may be selected using methods known in the art and described herein. For example, conventional binding assays and ubiquitination reactions with peptides derived from a putative low affinity binding site can be used to identify low affinity binding sites on cell cycle regulatory proteins. A peptide SPOTS blot technique may also be employed to identify binding of peptides derived from a putative low affinity binding site and an F-box protein, or part or complex thereof.

In this method of the invention, the sequence motif is optimized so that a peptide comprising the motif or peptide mimetic thereof, is capable of binding to an F-box protein with a high affinity. A high affinity interaction between a high affinity motif and an F-box protein typically has a K_{d} of less than 25 μM, and more preferably less than 1 μM, 100 nM or even 10 nM.

The sequence motif is optimized using methods known in the art and described herein. For example, a peptide SPOTS blot technique may be used to identify sequence motifs that bind with high affinity to an F-box protein, or part or complex thereof.

Peptides and peptide mimetics may be synthesized using techniques known to persons skilled in the art (see discussion below re CPD peptides).

An agent can be tested in in vivo or in vitro assays to ascertain if the agent modulates ubiquitin-dependent proteolysis of the protein. In an embodiment, the agent is tested in cellular assays or animal model assays. For example, ubiquitination reactions as described herein may be used to determine if an agent is a modulator.

In an embodiment, an agent is tested for its ability to affect the interaction between an F-box protein and a regulatory protein that interacts with the F-box protein comprising:

(a) exposing an agent to the F-box protein and regulatory protein for a sufficient time to allow the F-box protein and regulatory protein to interact;

(b) removing non-bound agent; and

(c) determining the presence of agent bound to the F-box protein and/or the regulatory protein thereby identifying an agent that affects the interaction.

The invention also contemplates the agents (e.g. motifs, peptides comprising the motifs, and peptide mimetics thereof) identified using this method of the invention. The agents (e.g. motifs, peptides comprising the motifs, and peptide mimetics thereof) may be used to modulate ubiquitin dependent proteolysis, and they may be used to modulate cellular processes of cells (such as proliferation, growth, and/or differentiation, in particular glucose and methionine biosynthesis, gene expression, cell division, and transcription) in which the agents are introduced. An agent may be used to disrupt ubiquitin-dependent proteolysis of a regulatory protein (ie. stabilize a regulatory protein), or to selectively degrade a target protein, for instance by fusing the motif to a binding partner of the target protein.

Accordingly, the agents (e.g. motifs, peptides comprising the motifs, and peptide mimetics thereof) may be formulated into compositions for administration to individuals suffering from a proliferative or differentiative condition. Therefore, the present invention also relates to a composition comprising an agent (e.g. motifs, peptides comprising the motifs, and peptide mimetics thereof), and a pharmaceutically acceptable carrier, excipient or diluent. A method for modulating proliferation, growth, and/or differentiation of cells is also provided comprising introducing into the cells an agent (e.g. motifs, peptides comprising the motifs, and peptide mimetics thereof) or a composition containing same. Methods for treating proliferative and/or differentiative conditions or diseases using the compositions of the invention are also provided.

Still further the invention provides the use of an agent in the preparation of a medicament to modulate ubiquitin-dependent proteolysis in cells of an individual. The invention also contemplates the use of an agent in the preparation of medicament to treat individuals suffering from a proliferative or differentiative condition.

CPD Peptides and Chimeric Proteins

The invention provides molecules derived from a CPD motif, or opitmized from a CPD motif.

In accordance with an embodiment of the invention, the molecules are CPD peptides derived from a CPD motif of cyclin E. In particular the invention provides CPD peptides of the formula:

X¹-X²-X³-pThr-Pro-X4-X⁸

wherein X¹ represents 0 to 100 amino acids, preferably 0 to 50, more preferably 0 to 20, most preferably 0 to 10 amino acids, X² represents Leu, Pro, or Ile, preferably Leu or Ile; X³ represents Leu, Ile, Val, or Pro, preferably Ile, Leu, or Pro; X⁴ represents any amino acid except basic and bulky hydrophobic amino acids, preferably X⁴ is any amino acid except Arg, Lys, or Tyr more preferably X4 is Ile, Val, Pro, or Gin, and X⁸ represents 0 to 100 amino acids, preferably 0 to 50, more preferably 0 to 20, most preferably 0 to 10 amino acids.

In accordance with an embodiment of the invention, the molecules are CPD peptides of the formula:

X¹-X²-X³-pThr-Pro-X⁴-X⁵-X⁶-X⁷-X⁸

wherein X¹ represents 0 to 100 amino acids, preferably 0 to 50, more preferably 0 to 20, most preferably 0 to 10 amino acids; X² represents Leu, Pro, or Ile, preferably Leu or Ile; X³ represents Leu, Ile, Val, or Pro, preferably Ile, Leu, or Pro; X⁴, X⁵ and X⁶ represent any amino acid except basic and bulky hydrophobic amino acids (e.g. Tyr), preferably X⁴ is any amino acid except Arg, Lys, Tyr, or Trp, more preferably X⁴ is Ile, Val, Pro, or GIn, preferably X⁵ and X⁶ are not Arg, Lys, or Tyr and more preferably X⁵ is GIn, Leu, Met, Thr, or Glu, and X⁶ is Gin, Ala, Thr, Glu, or Ser; X⁷ is any amino acid, preferably not a basic or bulky hydrophobic amino acid (e.g. Tyr), more preferably X⁷ is not Arg, Lys, or Tyr, most preferably X⁷ is Leu, Trp, Asp, Pro, or Gly; and X⁸ represents 0 to 100 amino acids, preferably 0 to 50, more preferably 0 to 20, most preferably 0 to 10 amino acids.

In accordance with a further embodiment of the invention the molecules are derived from a CPD motif of Gcn4. In particular, the invention provides CPD peptides of the formula:

X¹-Leu/Gly/Tyr-Pro-pThr-Pro-X⁹

wherein X¹ represents 0 to 100 amino acids, preferably 0 to 50, more preferably 0 to 20, most preferably 0 to 10 amino acids, and X⁹ represents 0 to 100 amino acids, preferably 0 to 50, more preferably 0 to 20, most preferably 0 to 10 amino acids, or represents X¹⁰-X¹¹-X¹²-X¹³-X¹⁴ wherein X¹⁰ is any amino acid except Arg, X¹¹ is any amino acid except Cys, X¹² is any amino acid except Arg, Cys, and Lys, X¹³ is any amino acid except Arg and Cys, and X¹⁴ represents 0 to 100 amino acids, preferably 0 to 50, more preferably 0 to 20, most preferably 0 to 10 amino acids.

In a preferred embodiment, a CPD peptide of the invention binds to a CPD motif binding partner with a K_{d} of less than 25 μM, and more preferably less than 1 μM, 100 nM or even 10 nM.

All of the peptides of the invention, as well as molecules substantially homologous, complementary or otherwise functionally or structurally equivalent to these peptides may be used for purposes of the present invention. In addition to full-length peptides of the invention, truncations of the peptides are contemplated in the present invention. Truncated peptides may comprise peptides of about 5 to 8 amino acid residues

The truncated peptides may have an amino group (—NH2), a hydrophobic group (for example, carbobenzoxyl, dansyl, or T-butyloxycarbonyl), an acetyl group, a 9-fluorenylmethoxy-carbonyl (PMOC) group, or a macromolecule including but not limited to lipid-fatty acid conjugates, polyethylene glycol, or carbohydrates at the amino terminal end. The truncated peptides may have a carboxyl group, an amido group, a T-butyloxycarbonyl group, or a macromolecule including but not limited to lipid-fatty acid conjugates, polyethylene glycol, or carbohydrates at the carboxy terminal end.

The peptides of the invention may also include analogs of a peptide of the invention, and/or truncations of the peptide, which may include, but are not limited to the peptide of the invention containing one or more amino acid insertions, additions, or deletions, or both. Analogs of a peptide of the invention exhibit the activity characteristic of the peptide, and may further possess additional advantageous features such as increased bioavailability, stability, or reduced host immune recognition.

One or more amino acid insertions may be introduced into a peptide of the invention. Amino acid insertions may consist of a single amino acid residue or sequential amino acids. One or more amino acids, preferably one to five amino acids, may be added to the right or left termini of a peptide of the invention. Deletions may consist of the removal of one or more amino acids, or discrete portions from the peptide sequence. The deleted amino acids may or may not be contiguous. The lower limit length of the resulting analog with a deletion mutation is about 7 amino acids.

It is anticipated that if amino acids are inserted or deleted in sequences outside a CPD consensus sequence that the resulting analog of the peptide will function to bind to an interacting or associated molecule such as an F-box Protein.

Preferred peptides of the invention include the following: ASPLPSGLLpTPPQSGKKQS (SEQ ID NO. 1), ASPLPSGLLpTPPQSGK (SEQ ID NO. 2), GLLpTPPQSG (SEQ ID NO. 3), TGEFPQFpTPQEQLI (SEQ ID NO. 4), LSKNLLpTPQEEWD (SEQ ID NO. 5), FLPpTPVLED (SEQ ID NO. 6), L/I-L/I/P-pT-P<RKY>₄ where < > refers to the disfavoured amino acid residues, XₙLLpTPPXₙ (SEQ ID NO. 7), XₙLLpTPILAXₙ (SEQ ID NO. 8), XₙPVpTPPMSPXₙ (SEQ ID NO. 9), XₙILpTPPTTXₙ (SEQ ID NO. 10), and XₙLIpTPPTTXₙ (SEQ ID NO. 11), where X is any amino acid and n may be 0 to 100 amino acids, preferably 0 to 50, more preferably 0 to 20, and most preferably 0 to 10. Additional preferred peptides include TSFLPpTPVLED (SEQ ID NO. 32); XₙLPpTPXₙ (SEQ ID NO 33), XₙGPpTPXₙ (SEQ ID NO. 34), and XₙYPpTPXₙ (SEQ ID NO. 35) where X is any amino acid and n may be 0 to 100 amino acids, preferably 0 to 50, more preferably 0 to 20, and most preferably 0 to 10.

The invention also encompasses molecules derived from CPD peptides of the invention, preferably molecules that interact with or bind to, or alter the function of the SCF complex, preferably a mammalian SCF complex.

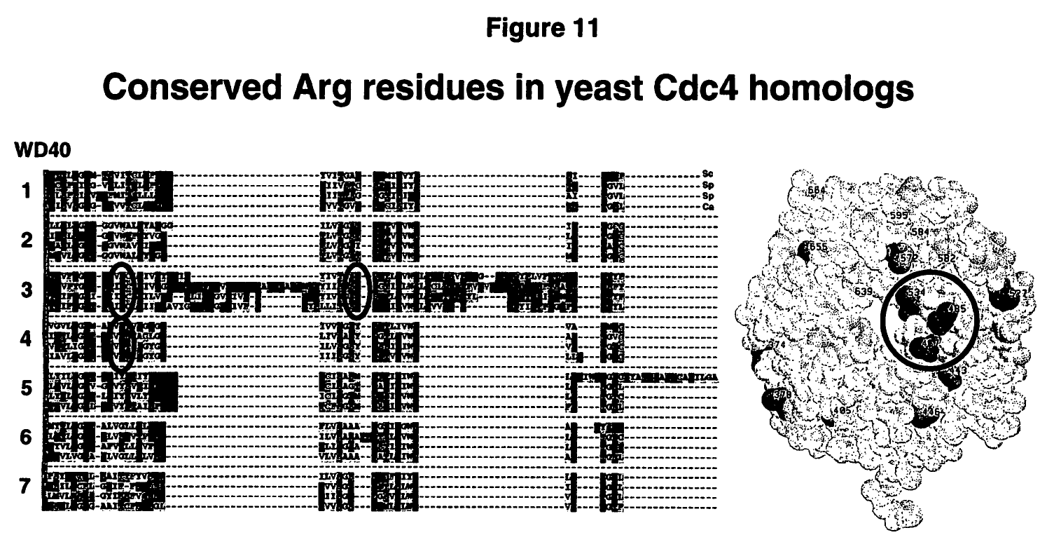

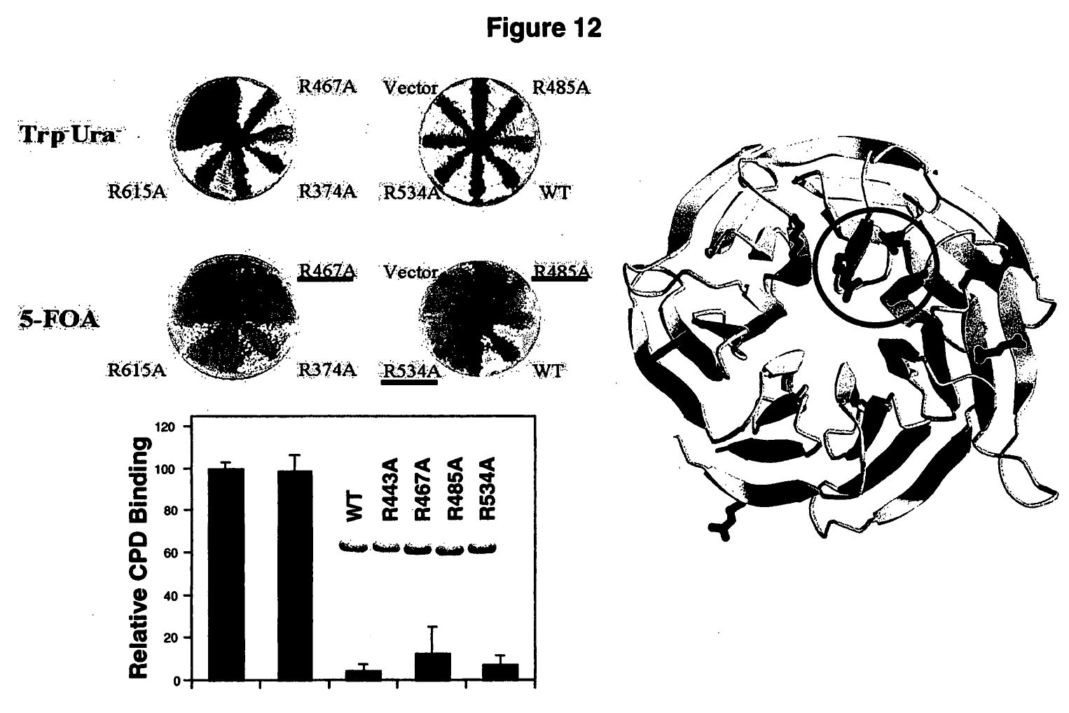

The invention also relates to molecules derived from a CPD motif binding partner, such as a binding domain of an F-box protein that binds a CPD motif. For example, a peptide or peptide mimetic can be prepared based on the binding domain for a CPD peptide of an F-box protein such as Cdc4. FIGS. 11 and 12 show the sequence and structure of a binding domain of Cdc4 which interacts with CPD peptides. Thus, a peptide could be prepared comprising the structure of such a binding domain of Cdc4 (preferably comprising amino acid residues Arg457, Arg485 and Arg534) as shown in FIG. 11 or FIG. 12.

The invention also relates to novel chimeric proteins comprising at least one CPD motif, or CPD peptide of the invention fused to, or integrated into, a target protein, and/or a targeting domain capable of directing the chimeric protein to a desired cellular component or cell type or tissue. The chimeric proteins may also contain additional amino acid sequences or domains. The chimeric proteins are recombinant in the sense that the various components are from different sources, and as such are not found together in nature (i.e. are heterologous).

A target protein is a protein that is selected for degradation and for example may be a protein that is mutated or over expressed in a disease or condition. The targeting domain can be a membrane spanning domain, a membrane binding domain, or a sequence directing the protein to associate with for example vesicles or with the nucleus. The targeting domain can target a CPD motif or CPD peptide to a particular cell type or tissue. For example, the targeting domain can be a cell surface ligand or an antibody against cell surface antigens of a target tissue (e.g. tumor antigens). A targeting domain may target a CPD motif or CPD peptide to a cellular component. For example, a targeting domain may be an SH2 or SH3 domain. Thus, the method of the invention may be used to target proteins that bind to an SH2 or SH3 domain for ubiquitin-dependent proteolysis.

A CPD motif, CPD peptide, CPD motif binding partner, or chimeric protein of the invention may be conjugated with other molecules, such as proteins, to prepare fusion proteins. This may be accomplished, for example, by the synthesis of N-terminal or C-terminal fusion proteins

A CPD motif, CPD peptide, CPD motif binding partner, or chimeric protein of the invention may be prepared using recombinant DNA methods. Accordingly, nucleic acid molecules which encode a CPD motif, CPD peptide, CPD motif bnding partner, or chimeric protein of the invention may be incorporated in a known manner into an appropriate expression vector which ensures good expression of the CPD motif, CPD peptide, CPD motif binding partner, or chimeric protein. Possible expression vectors include but are not limited to cosmids, plasmids, or modified viruses so long as the vector is compatible with the host cell used. The expression vectors contain a nucleic acid molecule encoding a CPD motif, CPD peptide, CPD motif binding partner, or chimeric protein of the invention and the necessary regulatory sequences for the transcription and translation of the inserted protein-sequence. Suitable regulatory sequences may be obtained from a variety of sources, including bacterial, fungal, viral, mammalian, or insect genes (For example, see the regulatory sequences described in Goeddel, Gene Expression Technology: Methods in Enzymology 185, Academic Press, San Diego, Calif. (1990). Selection of appropriate regulatory sequences is dependent on the host cell chosen, and may be readily accomplished by one of ordinary skill in the art. Other sequences, such as an origin of replication, additional DNA restriction sites, enhancers, and sequences conferring inducibility of transcription may also be incorporated into the expression vector.

The recombinant expression vectors may also contain a selectable marker gene which facilitates the selection of transformed or transfected host cells. Suitable selectable marker genes are genes encoding proteins such as G418 and hygromycin which confer resistance to certain drugs, β-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

The recombinant expression vectors may also contain nucleic acid molecules which encode a portion which provides increased expression of the recombinant CPD motif, CPD peptide, or chimeric protein; increased solubility of the recombinant CPD motif, peptide, CPD motif binding partner, or chimeric protein; and/or aid in the purification of the recombinant CPD motif, CPD peptide, CPD motif binding partner, or chimeric protein by acting as a ligand in affinity purification. For example, a proteolytic cleavage site may be inserted in the recombinant peptide to allow separation of the recombinant CPD motif, CPD peptide, CPD motif binding partner, or chimeric protein from the fusion portion after purification of the fusion protein. Examples of fusion expression vectors include pGEX (Amrad Corp., Melbourne, Australia), pMAL (New England Biolabs, Beverly, Mass.) and pRIT5 (Pharmacia, Piscataway, N.J.) which fuse glutathione S-transferase (GST), maltose E binding protein, or protein A, respectively, to the recombinant protein.

Recombinant expression vectors may be introduced into host cells to produce a transformant host cell. Transformant host cells include prokaryotic and eukaryotic cells which have been transformed or transfected with a recombinant expression vector of the invention. The terms “transformed with”, “transfected with”, “transformation” and “transfection” are intended to include the introduction of nucleic acid (e.g. a vector) into a cell by one of many techniques known in the art. For example, prokaryotic cells can be transformed with nucleic acid by electroporation or calcium-chloride mediated transformation. Nucleic acid can be introduced into mammalian cells using conventional techniques such as calcium phosphate or calcium chloride co-precipitation, DEAE-dextran-mediated transfection, lipofectin, electroporation or microinjection. Suitable methods for transforming and transfecting host cells may be found in Sambrook et al. (Molecular Cloning: A Laboratory Manual, 2nd Edition, Cold Spring Harbor Laboratory press (1989)), and other laboratory textbooks.

Suitable host cells include a wide variety of prokaryotic and eukaryotic host cells. For example, a CPD motif, CPD peptide, CPD motif binding partner, or chimeric protein of the invention may be expressed in bacterial cells such as E. coli, insect cells (using baculovirus), yeast cells or mammalian cells. Other suitable host cells can be found in Goeddel, Gene Expression Technology: Methods in Enzymology 185, Academic Press, San Diego, Calif. (1991).

A CPD motif, CPD peptide, CPD motif binding partner, or chimeric protein of the invention may be phosphorylated using conventional methods such as the method described in Reedijk et al. (The EMBO Journal 11(4):1365, 1992).

A CPD motif, CPD peptide, CPD motif binding partner, or chimeric protein of the invention may be synthesized by conventional techniques. For example, the peptides or chimeric proteins may be synthesized by chemical synthesis using solid phase peptide synthesis. These methods employ either solid or solution phase synthesis methods (see for example, J. M. Stewart, and J. D. Young, Solid Phase Peptide Synthesis, 2^{nd} Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis.) By way of example, a CPD motif, CPD peptide, CPD motif binding partner, or chimeric protein may be synthesized using 9-fluorenyl methoxycarbonyl (Fmoc) solid phase chemistry with direct incorporation of phosphothreonine as the N-fluorenylmethoxy-carbonyl-O-benzyl-L-phosphothreonine derivative.

N-terminal or C-terminal fusion proteins comprising a CPD motif, CPD peptide, CPD motif binding partner, or chimeric protein of the invention conjugated with other molecules may be prepared by fusing, through recombinant techniques, the N-terminal or C-terminal of the CPD motif, CPD peptide, CPD motif binding partner, or chimeric protein, and the sequence of a selected protein or selectable marker with a desired biological function. The resultant fusion proteins contain the CPD motif, CPD peptide, CPD motif binding partner, or chimeric protein fused to the selected protein or marker protein as described herein. Examples of proteins which may be used to prepare fusion proteins include immunoglobulins, glutathione-S-transferase (GST), hemagglutinin (HA), and truncated myc.

Cyclic derivatives of the peptides or chimeric proteins of the invention are also part of the present invention. Cyclization may allow the peptide or chimeric protein to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic peptide which is more flexible than the cyclic peptides containing peptide bond linkages as described above. A more flexible peptide may be prepared by introducing cysteines at the right and left position of the peptide and forming a disulphide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

Peptide mimetics may be designed based on information obtained by systematic replacement of L-amino acids by D-amino acids, replacement of side chains with groups having different electronic properties, and by systematic replacement of peptide bonds with amide bond replacements. Local conformational constraints can also be introduced to determine conformational requirements for activity of a candidate peptide mimetic. The mimetics may include isosteric amide bonds, or D-amino acids to stabilize or promote reverse turn conformations and to help stabilize the molecule. Cyclic amino acid analogues may be used to constrain amino acid residues to particular conformational states. The mimetics can also include mimics of inhibitor peptide secondary structures. These structures can model the 3-dimensional orientation of amino acid residues into the known secondary conformations of proteins. Peptoids may also be used which are oligomers of N-substituted amino acids and can be used as motifs for the generation of chemically diverse libraries of novel molecules.

Peptides of the invention may be developed using a biological expression system. The use of these systems allows the production of large libraries of random peptide sequences and the screening of these libraries for peptide sequences that bind to particular proteins. Libraries may be produced by cloning synthetic DNA that encodes random peptide sequences into appropriate expression vectors. (see Christian et al 1992, J. Mol. Biol. 227:711; Devlin et al, 1990 Science 249:404; Cwirla et al 1990, Proc. Natl. Acad, Sci. USA, 87:6378). Libraries may also be constructed by concurrent synthesis of overlapping peptides (see U.S. Pat. No. 4,708,871).

The peptides and chimeric proteins of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

The invention also contemplates antibodies specific for a CPD motif, CPD peptide, CPD motif binding partner, or chimeric protein of the invention. The antibodies may be intact monoclonal or polyclonal antibodies, and immunologically active fragments (e.g. a Fab or (Fab)₂ fragment), an antibody heavy chain, an antibody light chain, humanized antibodies, a genetically engineered single chain F_{V} molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Antibodies can be prepared using intact polypeptides or fragments containing an immunizing antigen of interest. The polypeptide or oligopeptide used to immunize an animal may be obtained from the translation of RNA or synthesized chemically and can be conjugated to a carrier protein, if desired. Suitable carriers that may be chemically coupled to peptides include bovine serum albumin and thyroglobulin, keyhole limpet hemocyanin. The coupled polypeptide may then be used to immunize the animal (e.g., a mouse, a rat, or a rabbit).

A CPD motif, CPD peptide, CPD motif binding partner, or chimeric protein, and antibodies specific for same may be labelled using conventional methods with various enzymes, fluorescent materials, luminescent materials and radioactive materials. Suitable enzymes, fluorescent materials, luminescent materials, and radioactive material are well known to the skilled artisan. Labeled antibodies specific for the peptides of the invention may be used to screen for proteins with a CPD motif, and a labeled CPD motif or peptide of the invention may be used to screen for proteins containing binding sites for a CPD motif (e.g. CPD motif binding partners).

Combined with certain formulations, such peptides can be effective intracellular agents. However, in order to increase the efficacy of such peptides, the CPD peptide can be provided a fusion peptide along with a second peptide which promotes “transcytosis”, e.g., uptake of the peptide by epithelial cells. To illustrate, the CPD peptide of the present invention can be provided as part of a fusion polypeptide with all or a fragment of the N-terminal domain of the HIV protein Tat, e.g., residues 1-72 of Tat or a smaller fragment thereof which can promote transcytosis. In other embodiments, the CPD peptide can be provided a fusion polypeptide with all or a portion of the antenopedia III protein.

To further illustrate, the CPD peptide (or peptidomimetic) can be provided as a chimeric peptide which includes a heterologous peptide sequence (“internalizing peptide”) which drives the translocation of an extracellular form of a CPD peptide sequence across a cell membrane in order to facilitate intracellular localization of the CPD peptide. In this regard, the therapeutic CPD binding sequence is one which is active intracellularly. The internalizing peptide, by itself, is capable of crossing a cellular membrane by, e.g., transcytosis, at a relatively high rate. The internalizing peptide is conjugated, e.g., as a fusion protein, to the CPD peptide. The resulting chimeric peptide is transported into cells at a higher rate relative to the activator polypeptide alone to thereby provide an means for enhancing its introduction into cells to which it is applied, e.g., to enhance topical applications of the CPD peptide.

In one embodiment, the internalizing peptide is derived from the Drosophila antennapedia protein, or homologs thereof. The 60 amino acid long long homeodomain of the homeo-protein antennapedia has been demonstrated to translocate through biological membranes and can facilitate the translocation of heterologous polypeptides to which it is couples. See for example Derossi et al. (1994) J Biol Chem 269:10444-10450; and Perez et al. (1992) J Cell Sci 102:717-722. Recently, it has been demonstrated that fragments as small as 16 amino acids long of this protein are sufficient to drive internalization. See Derossi et al. (1996) J Biol Chem 271:18188-18193.

The present invention contemplates a CPD peptide or peptidomimetic sequence as described herein, and at least a portion of the Antennapedia protein (or homolog thereof) sufficient to increase the transmembrane transport of the chimeric protein, relative to the CPD peptide or peptidomimetic, by a statistically significant amount.

Another example of an internalizing peptide is the HIV transactivator (TAT) protein. This protein appears to be divided into four domains (Kuppuswamy et al. (1989) Nucl. Acids Res. 17:3551-3561). Purified TAT protein is taken up by cells in tissue culture (Frankel and Pabo, (1989) Cell, 55:1189-1193), and peptides, such as the fragment corresponding to residues 37-62 of TAT, are rapidly taken up by cell in vitro (Green and Loewenstein, (1989) Cell 55:1179-1188). The highly basic region mediates internalization and targeting of the internalizing moiety to the nucleus (Ruben et al., (1989) J. Virol. 63:1-8).

Another exemplary transcellular polypeptide can be generated to include a sufficient portion of mastoparan (T. Higashijima et al., (1990) J. Biol. Chem. 265:14176) to increase the transmembrane transport of the chimeric protein.

While not wishing to be bound by any particular theory, it is noted that hydrophilic polypeptides may be also be physiologically transported across the membrane barriers by coupling or conjugating the polypeptide to a transportable peptide which is capable of crossing the membrane by receptor-mediated transcytosis. Suitable internalizing peptides of this type can be generated using all or a portion of, e.g., a histone, insulin, transferrin, basic albumin, prolactin and insulin-like growth factor I (IGF-I), insulin-like growth factor II (IGF-II) or other growth factors. For instance, it has been found that an insulin fragment, showing affinity for the insulin receptor on capillary cells, and being less effective than insulin in blood sugar reduction, is capable of transmembrane transport by receptor-mediated transcytosis and can therefor serve as an internalizing peptide for the subject transcellular peptides and peptidomimetics. Preferred growth factor-derived internalizing peptides include EGF (epidermal growth factor)-derived peptides, such as CMHIESLDSYTC (SEQ ID NO. 36) and CMYIEALDKYAC (SEQ ID NO. 37); TGF-beta (transforming growth factor beta)-derived peptides; peptides derived from PDGF (platelet-derived growth factor) or PDGF-2; peptides derived from IGF-I (insulin-like growth factor) or IGF-II; and FGF (fibroblast growth factor)-derived peptides.

Another class of translocating/internalizing peptides exhibits pH-dependent membrane binding. For an internalizing peptide that assumes a helical conformation at an acidic pH, the internalizing peptide acquires the property of amphiphilicity, e.g., it has both hydrophobic and hydrophilic interfaces. More specifically, within a pH range of approximately 5.0-5.5, an internalizing peptide forms an alpha-helical, amphiphilic structure that facilitates insertion of the moiety into a target membrane. An alpha-helix-inducing acidic pH environment may be found, for example, in the low pH environment present within cellular endosomes. Such internalizing peptides can be used to facilitate transport of CPD peptides and peptidomimetics, taken up by an endocytic mechanism, from endosomal compartments to the cytoplasm.

A preferred pH-dependent membrane-binding internalizing peptide includes a high percentage of helix-forming residues, such as glutamate, methionine, alanine and leucine. In addition, a preferred internalizing peptide sequence includes ionizable residues having pKa's within the range of pH 5-7, so that a sufficient uncharged membrane-binding domain will be present within the peptide at pH 5 to allow insertion into the target cell membrane.

A particularly preferred pH-dependent membrane-binding internalizing peptide in this regard is aa1-aa2-aa3-EAALA(EALA)4-EALEALAA-amide (SEQ ID NO. 38), which represents a modification of the peptide sequence of Subbarao et al. (Biochemistry 26:2964, 1987). Within this peptide sequence, the first amino acid residue (aa1) is preferably a unique residue, such as cysteine or lysine, that facilitates chemical conjugation of the internalizing peptide to a targeting protein conjugate. Amino acid residues 2-3 may be selected to modulate the affinity of the internalizing peptide for different membranes. For instance, if both residues 2 and 3 are lys or arg, the internalizing peptide will have the capacity to bind to membranes or patches of lipids having a negative surface charge. If residues 2-3 are neutral amino acids, the internalizing peptide will insert into neutral membranes.

Yet other preferred internalizing peptides include peptides of apo-lipoprotein A-1 and B; peptide toxins, such as melittin, bombolittin, delta hemolysin and the pardaxins; antibiotic peptides, such as alamethicin; peptide hormones, such as calcitonin, corticotrophin releasing factor, beta endorphin, glucagon, parathyroid hormone, pancreatic polypeptide; and peptides corresponding to signal sequences of numerous secreted proteins. In addition, exemplary internalizing peptides may be modified through attachment of substituents that enhance the alpha-helical character of the internalizing peptide at acidic pH.

Yet another class of internalizing peptides suitable for use within the present invention include hydrophobic domains that are “hidden” at physiological pH, but are exposed in the low pH environment of the target cell endosome. Upon pH-induced unfolding and exposure of the hydrophobic domain, the moiety binds to lipid bilayers and effects translocation of the covalently linked polypeptide into the cell cytoplasm. Such internalizing peptides may be modeled after sequences identified in, e.g., Pseudomonas exotoxin A, clathrin, or Diphtheria toxin.

Pore-forming proteins or peptides may also serve as internalizing peptides herein. Pore-forming proteins or peptides may be obtained or derived from, for example, C9 complement protein, cytolytic T-cell molecules or NK-cell molecules. These moieties are capable of forming ring-like structures in membranes, thereby allowing transport of attached polypeptide through the membrane and into the cell interior.

Mere membrane intercalation of an internalizing peptide may be sufficient for translocation of the CPD peptide or peptidomimetic, across cell membranes. However, translocation may be improved by attaching to the internalizing peptide a substrate for intracellular enzymes (i.e., an “accessory peptide”). It is preferred that an accessory peptide be attached to a portion(s) of the internalizing peptide that protrudes through the cell membrane to the cytoplasmic face. The accessory peptide may be advantageously attached to one terminus of a translocating/internalizing moiety or anchoring peptide. An accessory moiety of the present invention may contain one or more amino acid residues. In one embodiment, an accessory moiety may provide a substrate for cellular phosphorylation (for instance, the accessory peptide may contain a tyrosine residue).

An exemplary accessory moiety in this regard would be a peptide substrate for N-myristoyl transferase, such as GNAAAARR (SEQ ID NO. 39) (Eubanks et al., in: Peptides, Chemistry and Biology, Garland Marshall (ed.), ESCOM, Leiden, 1988, pp. 566-69) In this construct, an internalizing peptide would be attached to the C-terminus of the accessory peptide, since the N-terminal glycine is critical for the accessory moiety's activity. This hybrid peptide, upon attachment to an E2 peptide or peptidomimetic at its C-terminus, is N-myristylated and further anchored to the target cell membrane, e.g., it serves to increase the local concentration of the peptide at the cell membrane.

To further illustrate use of an accessory peptide, a phosphorylatable accessory peptide is first covalently attached to the C-terminus of an internalizing peptide and then incorporated into a fusion protein with a CPD peptide or peptidomimetic. The peptide component of the fusion protein intercalates into the target cell plasma membrane and, as a result, the accessory peptide is translocated across the membrane and protrudes into the cytoplasm of the target cell. On the cytoplasmic side of the plasma membrane, the accessory peptide is phosphorylated by cellular kinases at neutral pH. Once phosphorylated, the accessory peptide acts to irreversibly anchor the fusion protein into the membrane. Localization to the cell surface membrane can enhance the translocation of the polypeptide into the cell cytoplasm.

Suitable accessory peptides include peptides that are kinase substrates, peptides that possess a single positive charge, and peptides that contain sequences which are glycosylated by membrane-bound glycotransferases. Accessory peptides that are glycosylated by membrane-bound glycotransferases may include the sequence x-NLT-x, where “x” may be another peptide, an amino acid, coupling agent or hydrophobic molecule, for example. When this hydrophobic tripeptide is incubated with microsomal vesicles, it crosses vesicular membranes, is glycosylated on the luminal side, and is entrapped within the vesicles due to its hydrophilicity (C. Hirschberg et al., (1987) Ann. Rev. Biochem. 56:63-87). Accessory peptides that contain the sequence x-NLT-x thus will enhance target cell retention of corresponding polypeptide.

In another embodiment of this aspect of the invention, an accessory peptide can be used to enhance interaction of the CPD peptide or peptidomimetic with the target cell. Exemplary accessory peptides in this regard include peptides derived from cell adhesion proteins containing the sequence “RGD”, or peptides derived from laminin containing the sequence CDPGYIGSRC (SEQ ID NO. 40). Extracellular matrix glycoproteins, such as fibronectin and laminin, bind to cell surfaces through receptor-mediated processes. A tripeptide sequence, RGD, has been identified as necessary for binding to cell surface receptors. This sequence is present in fibronectin, vitronectin, C3bi of complement, von-Willebrand factor, EGF receptor, transforming growth factor beta, collagen type I, lambda receptor of E. Coli, fibrinogen and Sindbis coat protein (E. Ruoslahti, Ann. Rev. Biochem. 57:375413, 1988). Cell surface receptors that recognize RGD sequences have been grouped into a superfamily of related proteins designated “integrins”. Binding of “RGD peptides” to cell surface integrins will promote cell-surface retention, and ultimately translocation, of the polypeptide.

As described above, the internalizing and accessory peptides can each, independently, be added to the CPD peptide or peptidomimetic by either chemical cross-linking or in the form of a fusion protein. In the instance of fusion proteins, unstructured polypeptide linkers can be included between each of the peptide moieties.

In general, the internalization peptide will be sufficient to also direct export of the polypeptide. However, where an accessory peptide is provided, such as an RGD sequence, it may be necessary to include a secretion signal sequence to direct export of the fusion protein from its host cell. In preferred embodiments, the secretion signal sequence is located at the extreme N-terminus, and is (optionally) flanked by a proteolytic site between the secretion signal and the rest of the fusion protein.

In an exemplary embodiment, a CPD peptide or peptidomimietic is engineered to include an integrin-binding RGD peptide/SV40 nuclear localization signal (see, for example Hart S L et al., 1994; J. Biol. Chem., 269:12468-12474), such as encoded by the nucleotide sequence provided in the Nde1-EcoR1 fragment: catatgggtggctgccgtggcgatatgttcggttgcggtgctcctccaaaaaagaagagaaag-gtagctggattc (SEQ ID NO. 41), which encodes the RGD/SV40 nucleotide sequence: MGGCRGDMFGCGAPP-KKKRKVAGF (SEQ ID NO. 42). In another embodiment, the protein can be engineered with the HIV-1 tat(1-72) polypeptide, e.g., as provided by the Nde1-EcoR1 fragment: catatggagccagtagatcctagactagagccc-tggaagcatccaggaagtcagcctaaaactgcttgtaccaattgctattgtaaaaagtgttgccattgccaagtttgtttcataacaaaagcccttggcatctcctatggcaggaagaagcggagacagcgacgaagactcctcaaggcagtcagactcatcaagtttctctaagtaagcaaggattc, which encodes the HIV-1 tat(1-72) peptide sequence: MEPVDPRLEPWKHPGSQPKT-ACTNCYCKKCCFHCQVCFITKALGISYGRKKRRQRRRPPQGSQTHQVSLSKQ (SEQ ID NO. 43). In still another embodiment, the fusion protein includes the HSV-1 VP22 polypeptide (Elliott G., O'Hare P (1997) Cell, 88:223-233) provided by the Nde1-EcoR1 fragment:

cat atg acc tct cgc cgc tcc gtg aag tcg ggt ccg cgg gag gtt ccg cgc gat gag tac gag gat ctg tac tac acc ccg tct tca ggt atg gcg agt ccc gat agt ccg cct gac acc tcc cgc cgt ggc gcc cta cag aca cgc tcg cgc cag agg ggc gag gtc cgt ttc gtc cag tac gac gag tcg gat tat gcc ctc tac ggg ggc tcg tca tcc gaa gac gac gaa cac ccg gag gtc ccc cgg acg cgg cgt ccc gtt tcc ggg gcg gtt ttg tcc ggc ccg ggg cct gcg cgg gcg cct ccg cca ccc gct ggg tcc gga ggg gcc gga cgc aca ccc acc acc gcc ccc cgg gcc ccc cga acc cag cgg gtg gcg act aag gcc ccc gcg gcc ccg gcg gcg gag acc acc cgc ggc agg aaa tcg gcc cag cca gaa tcc gcc gca ctc cca gac gcc ccc gcg tcg acg gcg cca acc cga tcc aag aca ccc gcg cag ggg ctg gcc aga aag ctg cac ttt agc acc gcc ccc cca aac ccc gac gcg cca tgg acc ccc cgg gtg gcc ggc ttt aac aag cgc gtc ttc tgc gcc gcg gtc ggg cgc ctg gcg gcc atg cat gcc cgg atg gcg gcg gtc cag ctc tgg gac atg tcg cgt ccg cgc aca gac gaa gac ctc aac gaa ctc ctt ggc atc acc acc atc cgc gtg acg gtc tgc gag ggc aaa aac ctg ctt cag cgc gcc aac gag ttg gtg aat cca gac gtg gtg cag gac gtc gac gcg gcc acg gcg act cga ggg cgt tet gcg gcg tcg cgc ccc acc gag cga cct cga gca cca gcc cgc tcc get tct cgc ccc aga cgg ccc gtc gag gaa ttc (SEQ ID NO. 44)

which encodes the HSV-1 VP22 peptide having the sequence:

MTSRRSVKSGPREVPRDEYEDLYYTPSSGMASPDSPPDTSRRGALQTRSRQRGEVRFVQYDESDYALYGGSSSEDDEHPEVPRTRRPVSGAVLSGPGPARAPPPPAGSGGAGRTPTTAPRAPRTGRVATKAPAAPAAETTRGRKSAQPESAALPDAPASTAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPRRPVE (SEQ ID NO. 45)

In still another embodiment, the fusion protein includes the C-terminal domain of the VP22 protein from, e.g., the nucleotide sequence (Nde1-EcoR1 fragment):

cat atg gac gtc gac gcg gcc acg gcg act cga ggg cgt tct gcg gcg tcg cgc ccc acc gag cga cct cga gcc cca gcc cgc tcc gct tct cgc ccc aga cgg ccc gtc gag gaa ttc (SEQ ID NO. 46)

which encodes the VP22 (C-terminal domain) peptide sequence: MDVDAATATRGRSA-ASRPTERPRAPARSASRPRRPVE (SEQ ID NO.47)

In certain instances, it may also be desirable to include a nuclear localization signal as part of the CPD peptide.

In the generation of fusion polypeptides including the subject CPD peptides, it may be necessary to include unstructured linkers in order to ensure proper folding of the various peptide domains. Many synthetic and natural linkers are known in the art and can be adapted for use in the present invention, including the (Gly₃Ser)₄ linker.

CPD Mimetics

In other embodiments, the subject CPD therapeutics are peptidomimetics of the CPD peptide. Peptidomimetics are compounds based on, or derived from, peptides and proteins. The CPD peptidomimetics of the present invention typically can be obtained by structural modification of a known CPD peptide sequence using unnatural amino acids, conformational restraints, isosteric replacement, and the like. The subject peptidomimetics constitute the continum of structural space between peptides and non-peptide synthetic structures; CPD peptidomimetics may be useful, therefore, in delineating pharmacophores and in helping to translate peptides into nonpeptide compounds with the activity of the parent CPD peptides.

Moreover, as is apparent from the present disclosure, mimetopes of the subject CPD peptides can be provided. Such peptidomimetics can have such attributes as being non-hydrolyzable (e.g., increased stability against proteases or other physiological conditions which degrade the corresponding peptide), increased specificity and/or potency, and increased cell permeability for intracellular localization of the peptidomimetic. For illustrative purposes, peptide analogs of the present invention can be generated using, for example, benzodiazepines (e.g., see Freidinger et al. in Peptides: Chemistry and Biology, G. R. Marshall ed., ESCOM Publisher: Leiden, Netherlands, 1988), substituted gama lactam rings (Garvey et al. in Peptides: Chemistry and Biology, G. R. Marshall ed., ESCOM Publisher: Leiden, Netherlands, 1988, p123), C-7 mimics (Huffman et al. in Peptides: Chemistry and Biologyy, G. R. Marshall ed., ESCOM Publisher: Leiden, Netherlands, 1988, p. 105), keto-methylene pseudopeptides (Ewenson et al. (1986) J Med Chem 29:295; and Ewenson et al. in Peptides: Structure and Function (Proceedings of the 9th American Peptide Symposium) Pierce Chemical Co. Rockland, Ill., 1985), β-turn dipeptide cores (Nagai et al. (1985) Tetrahedron Lett 26:647; and Sato et al. (1986) J Chem Soc Perkin Trans 1:1231), β-aminoalcohols (Gordon et al. (1985) Biochem Biophys Res Commun 126:419; and Dann et al. (1986) Biochem Biophys Res Commun 134:71), diaminoketones (Natarajan et al. (1984) Biochem Biophys Res Commun 124:141), and methyleneamino-modifed (Roark et al. in Peptides: Chemistry and Biology, G. R. Marshall ed., ESCOM Publisher: Leiden, Netherlands, 1988, p134). Also, see generally, Session III: Analytic and synthetic methods, in in Peptides: Chemistry and Biology, G. R. Marshall ed., ESCOM Publisher: Leiden, Netherlands, 1988)

In addition to a variety of sidechain replacements which can be carried out to generate the subject CPD peptidomimetics, the present invention specifically contemplates the use of conformationally restrained mimics of peptide secondary structure. Numerous surrogates have been developed for the amide bond of peptides. Frequently exploited surrogates for the amide bond include the following groups (i) trans-olefins, (ii) fluoroalkene, (iii) methyleneamino, (iv) phosphonamides, and (v) sulfonamides.

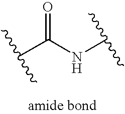

Examples of Surrogates

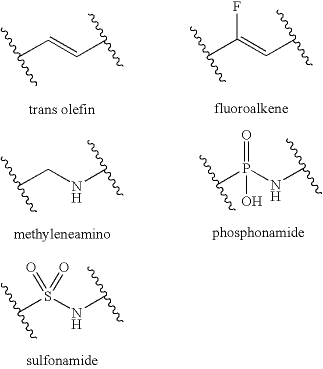

Additionally, peptidomimietics based on more substantial modifications of the backbone of the CPD peptide can be used. Peptidomimetics which fall in this category include (i) retro-inverso analogs, and (ii) N-alkyl glycine analogs (so-called peptoids).

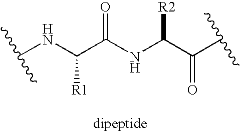

Examples of Analogs

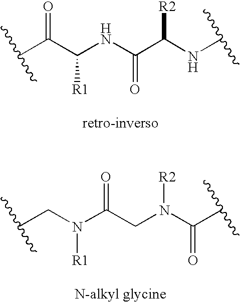

Furthermore, the methods of combinatorial chemistry are being brought to bear, c.f. Verdine et al. PCT publication WO9948897, on the development of new peptidomimetics. For example, one embodiment of a so-called “peptide morphing” strategy focuses on the random generation of a library of peptide analogs that comprise a wide range of peptide bond substitutes.

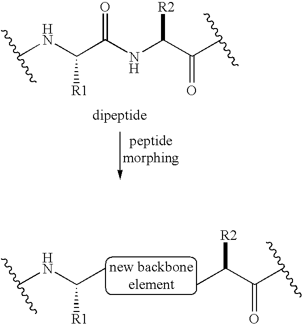

In an exemplary embodiment, the peptidomimetic can be derived as a retro-inverso analog of the peptide.

Retro-inverso analogs can be made according to the methods known in the art, such as that described by the Sisto et al. U.S. Pat. No. 4,522,752. As a general guide, sites which are most susceptible to proteolysis are typically altered, with less susceptible amide linkages being optional for mimetic switching. The final product, or intermediates thereof, can be purified by HPLC.

In another illustrative embodiment, the peptidomimetic can be derived as a retro-enatio analog of the peptide, such as the exemplary retro-enatio peptide analog derived for the illustrative LLpTPP peptide:

NH₂-Pro-Pro-(d)phosphoTyrosine-d)Leu-(d)Leu-COOH

Retro-enantio analogs such as this can be synthesized commercially available D-amino acids (or analogs thereof) and standard solid- or solution-phase peptide-synthesis techniques. For example, in a preferred solid-phase synthesis method, a suitably amino-protected (t-butyloxycarbonyl, Boc) D-phosphotyrosine residue (or analog thereof) is covalently bound to a solid support such as chloromethyl resin. The resin is washed with dichloromethane (DCM), and the BOC protecting group removed by treatment with TFA in DCM. The resin is washed and neutralized, and the next Boc-protected D-amino acid (D-Leu) is introduced by coupling with diisopropylcarbodiimide. The resin is again washed, and the cycle repeated for each of the remaining amino acids in turn. When synthesis of the protected retro-enantio peptide is complete, the protecting groups are removed and the peptide cleaved from the solid support by treatment with hydrofluoric acid/anisole/dimethyl sulfide/thioanisole. The final product is purified by HPLC to yield the pure retro-enantio analog.

In still another illustrative embodiment, trans-olefin derivatives can be made for any of the subject polypeptides. A trans olefin analog of CPD peptide can be synthesized according to the method of Y. K. Shue et al. (1987) Tetrahedron Letters 28:3225 and also according to other methods known in the art. It will be appreciated that variations in the cited procedure, or other procedures available, may be necessary according to the nature of the reagent used. It is further possible couple the pseudodipeptides synthesized by the above method to other pseudodipeptides, to make peptide analogs with several olefinic functionalities in place of amide functionalities.

Still another class of peptidomimetic derivatives include phosphonate derivatives. The synthesis of such phosphonate derivatives can be adapted from known synthesis schemes. See, for example, Loots et al. in Peptides: Chemistry and Biology, (Escom Science Publishers, Leiden, 1988, p. 118); Petrillo et al. in Peptides: Structure and Function (Proceedings of the 9th American Peptide Symposium, Pierce Chemical Co. Rockland, Ill., 1985).

Many other peptidomimetic structures are known in the art and can be readily adapted for use in the the subject CPD peptidomimetics. To illustrate, the CPD peptidomimetic may incorporate the 1-azabicyclo[4.3.0]nonane surrogate (see Kim et al. (1997) J. Org. Chem. 62:2847), or an N-acyl piperazic acid (see Xi et al. (1998) J. Am. Chem. Soc. 120:80), or a 2-substituted piperazine moiety as a constrained amino acid analogue (see Williams et al. (1996) J. Med. Chem. 39:1345-1348). In still other embodiments, certain amino acid residues can be replaced with aryl and bi-aryl moieties, e.g., monocyclic or bicyclic aromatic or heteroaromatic nucleus, or a biaromatic, aromatic-heteroaromatic, or biheteroaromatic nucleus.

The subject CPD peptidomimetics can be optimized by, e.g., combinatorial synthesis techniques combined with such high throughput screening as described herein.

Moreover, the phosphotyrosine can be replaced with analog, e.g., which is resistant to hydrolysis. Exemplary phosphotyrosine analogs include sidechains represented by the general formula:

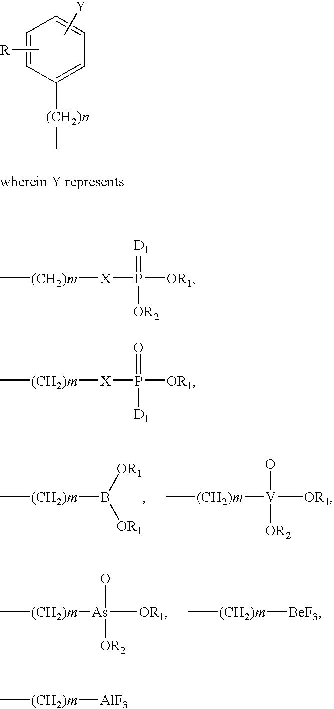

R1 and R2, independently for each occurrence, represent hydrogen, a lower alkyl, or a pharmaceutically acceptable salt; taken together with the atoms to which they are attached complete a heterocyclic ring having from 5 to 8 atoms in the ring structure;

D₁ represents O or S;

D₂ represents N₃, SH₂, NH₂, or NO₂;

m is 1,2, 3 or 4; and

n is 1, 1, 2 or 3.

Moreover, other examples of mimetopes include, but are not limited to, protein-based compounds, carbohydrate-based compounds, lipid-based compounds, nucleic acid-based compounds, natural organic compounds, synthetically derived organic compounds, anti-idiotypic antibodies and/or catalytic antibodies, or fragments thereof. A mimetope can be obtained by, for example, screening libraries of natural and synthetic compounds for compounds capable of binding to the CPD domain or inhibiting the interaction between the CPD domain and the natural ligand. A mimetope can also be obtained, for example, from libraries of natural and synthetic compounds, in particular, chemical or combinatorial libraries (i.e., libraries of compounds that differ in sequence or size but that have the same building blocks). A mimetope can also be obtained by, for example, rational drug design. In a rational drug design procedure, the three-dimensional structure of a compound of the present invention can be analyzed by, for example, nuclear magnetic resonance (NMR) or x-ray crystallography. The three-dimensional structure can then be used to predict structures of potential mimetopes by, for example, computer modelling, the predicted mimetope structures can then be produced by, for example, chemical synthesis, recombinant DNA technology, or by isolating a mimetope from a natural source (e.g., plants, animals, bacteria and fungi).

Complexes of the Invention

The invention contemplates a complex comprising a CPD motif and a CPD motif binding partner or substance that binds to a CPD motif, including an F-box Protein. It will be appreciated that the complex may comprise only the binding domains of the interacting molecules and such other flanking sequences as are necessary to maintain the activity of the complex. In an embodiment of the invention a complex is provided comprising a CPD motif of CyclinE and a CPD motif binding partner, preferably Cdc4.

The invention also contemplates antibodies specific for a complex of the invention. The antibodies may be intact monoclonal or polyclonal antibodies, and immunologically active fragments (e.g. a Fab or (Fab)₂ fragment), humanized antibodies, an antibody heavy chain, and antibody light chain, a genetically engineered single chain F_{V} molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Antibodies specific for a complex of the invention may be used to detect the complex in tissues and to determine their tissue distribution. In vitro and in situ detection methods using the antibodies of the invention may be used to assist in the prognostic and/or diagnostic evaluation of proliferative and/or differentiative disorders. Antibodies specific for a complex of the invention may also be used therapeutically to decrease the degradation of proteins that interact with CPD motif containing proteins, including F-box Proteins preferably WD40-repeat proteins.

A complex of the invention plays a role in ubiquitin-dependent proteolysis and some genetic diseases may include mutations at the binding domain regions of the interacting molecules in a complex of the invention. Therefore, if a complex of the invention is implicated in a genetic disorder, it may be possible to use PCR to amplify DNA from the binding domains to quickly check if a mutation is contained within one of the domains, in particular a CPD motif. Primers can be made corresponding to the flanking regions of the domains and standard sequencing methods can be employed to determine whether a mutation is present. This method does not require prior chromosome mapping of the affected gene and can save time by obviating sequencing the entire gene encoding a defective protein.

Methods for Identifying or Evaluating Substances/Compounds

The methods described herein are designed to screen or identify substances that modulate the activity of a CPD motif, CPD motif containing protein, CPD motif binding partner (e.g. F-box Protein), SCF complex, or complex of the invention, thus affecting ubquitin dependent proteolysis. Novel substances are therefore contemplated that interact with or bind to a CPD motif, a CPD motif binding partner, or complex of the invention, or bind to other proteins that interact with the molecules or complex, to compounds that interfere with, or enhance the interaction of molecules through a CPD motif or CPD motif binding partner, or other proteins that interact with the molecules.

The substances and compounds identified using the methods of the invention include but are not limited to peptides such as soluble peptides including Ig-tailed fusion peptides, members of random peptide libraries and combinatorial chemistry-derived molecular libraries made of D- and/or L-configuration amino acids, polysaccharides, oligosaccharides, monosaccharides, phosphopeptides (including members of random or partially degenerate, directed phosphopeptide libraries), antibodies [e.g. polyclonal, monoclonal, humanized, anti-idiotypic, chimeric, single chain antibodies, fragments, (e.g. Fab, F(ab)₂, and Fab expression library fragments, and epitope-binding fragments thereof)], and small organic or inorganic molecules. The substance or compound may be an endogenous physiological compound or it may be a natural or synthetic compound.

Substances which modulate the activity of a CPD motif, CPD motif containing protein, CPD motif binding partner, molecule derived from a CPD motif, or complex of the invention can be identified based on their ability to interact with or bind to a CPD motif, CPD motif containing protein, CPD motif binding partner, molecule derived from a CPD motif, or complex of the invention. Therefore, the invention also provides methods for identifying novel substances which bind a CPD motif, CPD motif containing protein, CPD motif binding partner, molecule derived from a CPD motif, or complex of the invention. Substances identified using the methods of the invention may be isolated, cloned and sequenced using conventional techniques.

Novel substances which can bind with a CPD motif (including a CPD motif in a CPD motif containing protein), CPD motif binding partner (preferably a sequence that interacts with a CPD motif), or a molecule in a complex of the invention may be identified by reacting a CPD motif, CPD motif binding partner, or molecule with at least one test substance which potentially interacts with or binds to a CPD motif, CPD motif binding partner, or molecule under conditions which permit the formation of complexes between the substance and CPD motif, CPD motif binding partner, or molecule, and removing and/or detecting the complexes. The detection of complexes indicates the substance binds to the CPD motif, CPD motif binding partner, or molecule. The complexes can be detected by assaying for substance-molecule complexes, for free substance, or for non-complexed CPD motif, CPD motif binding partner, or molecules. Conditions which permit the formation of complexes may be selected having regard to factors such as the nature and amounts of the substance and the CPD motif, CPD motif binding partner, or molecule.

The complexes, free substance, or non-complexed molecules may be isolated by conventional isolation techniques, for example, salting out, chromatography, electrophoresis, gel filtration, fractionation, absorption, polyacrylamide gel electrophoresis, agglutination, or combinations thereof. To facilitate the assay of the components, antibody against the CPD motif, CPD motif binding partner, molecule or the substance, or labelled CPD motif, CPD motif binding partner, or molecule, or a labelled substance may be utilized. The antibodies, motifs, binding partners, molecules, or substances may be labelled with a detectable substance as described above.

A CPD motif, CPD motif binding partner, molecule, or complex of the invention, or the substance used in the method of the invention may be insolubilized. For example, a motif, binding partner, molecule, or substance may be bound to a suitable carrier such as agarose, cellulose, dextran, Sephadex, Sepharose, carboxymethyl cellulose polystyrene, filter paper, ion-exchange resin, plastic film, plastic tube, glass beads, polyamine-methyl vinyl-ether-maleic acid copolymer, amino acid copolymer, ethylene-maleic acid copolymer, nylon, silk, etc. The carrier may be in the shape of, for example, a tube, test plate, beads, disc, sphere etc. The insolubilized protein or substance may be prepared by reacting the material with a suitable insoluble carrier using known chemical or physical methods, for example, cyanogen bromide coupling. It is possible to screen for agents that can be tested for their ability to treat a disease or condition characterized by an abnormality in a signal transduction pathway by testing compounds for their ability to affect the interaction between a CPD motif and a CPD motif binding partner, wherein the complex formed by such interaction is part of the signal transduction pathway.

The association or interaction between a CPD motif and a CPD motif binding partner may be promoted or enhanced either by increasing production of a CPD motif or CPD motif binding partner, or by increasing expression of a CPD motif or CPD motif binding partner, or by promoting interaction of a CPD motif and a CPD motif binding partner or by prolonging the duration of the association or interaction. The association or interaction between a CPD motif and a CPD motif binding partner, may be disrupted or reduced by preventing production of a CPD motif or CPD motif binding partner, or by preventing expression of a CPD motif or CPD motif binding partner, or by preventing interaction of a CPD motif and a CPD motif binding partner, or interfering with the interaction. A method may include measuring or detecting various properties including the level of signal transduction and the level of interaction between a CPD motif and a CPD motif binding partner. Depending upon the type of interaction present, various methods may be used to measure the level of interaction. For example, the strengths of covalent bonds are often measured in terms of the energy required to break a certain number of bonds (i.e., kcal/mol). Non-covalent interactions are often described as above, and also in terms of the distance between the interacting molecules. Indirect interactions may be described in a number of ways, including the number of intermediary agents involved, or the degree of control exercised over the CPD motif relative to the control exercised over the CPD motif binding partner.

The invention contemplates a method for evaluating a compound for its ability to modulate the biological activity of a complex of the invention (e.g. CPD motif and CPD motif binding protein preferably an F-box Protein; more preferably a CPD motif of cyclinE1 and a WD40-repeat protein such as cdc4), by assaying for an agonist or antagonist (i.e. enhancer or inhibitor) of the binding of molecules in the complex through the CPD motif. A basic method for evaluating if a compound is an agonist or antagonist of the binding of molecules in a complex of the invention, is to prepare a reaction mixture containing molecules and the substance under conditions which permit the formation of complexes, in the presence of a test compound. The test compound may be initially added to the mixture, or may be added subsequent to the addition of molecules. Control reaction mixtures without the test compound or with a placebo are also prepared. The formation of complexes is detected and the formation of complexes in the control reaction but not in the reaction mixture indicates that the test compound interferes with the interaction of the molecules. The reactions may be carried out in the liquid phase or the molecules, or test compound may be immobilized as described herein. The ability of a compound to modulate the biological activity of a CPD motif, CPD motif binding partner, or complex of the invention may be tested by determining the biological effects on cells or organisms using techniques known in the art.

It will be understood that the agonists and antagonists i.e. inhibitors and enhancers, that can be assayed using the methods of the invention may act on one or more of the binding sites on the interacting molecules in a complex including agonist binding sites, competitive antagonist binding sites, non-competitive antagonist binding sites or allosteric sites.

The invention also makes it possible to screen for antagonists that inhibit the effects of an agonist of the interaction of molecules in a complex of the invention. Thus, the invention may be used to assay for a compound that competes for the same binding site of a molecule in a complex of the invention.

The invention also contemplates methods for identifying novel compounds that interact with or bind to proteins that interact with a molecule of a complex of the invention. Protein-protein interactions may be identified using conventional methods such as co-immunoprecipitation, crosslinking and co-purification through gradients or chromatographic columns. Methods may also be employed that result in the simultaneous identification of genes which encode proteins interacting with a molecule. These methods include probing expression libraries with labeled molecules. Additionally, x-ray crystallographic studies may be used as a means of evaluating interactions with substances and molecules. For example, purified recombinant molecules in a complex of the invention when crystallized in a suitable form are amenable to detection of intra-molecular interactions by x-ray crystallography. Spectroscopy may also be used to detect interactions and in particular, Q-TOF instrumentation may be used. Two-hybrid systems may also be used to detect protein interactions in vivo.

It will be appreciated that fusion proteins and recombinant fusion proteins may be used in the above-described methods. For example, a CPD motif fused to a glutathione-S-transferase may be used in the methods.

It will also be appreciated that the complexes of the invention may be reconstituted in vitro using recombinant molecules and the effect of a test substance may be evaluated in the reconstituted system.

The reagents suitable for applying the methods of the invention to evaluate substances and compounds that modulate ubiquitin dependent proteolysis may be packaged into convenient kits providing the necessary materials packaged into suitable containers. The kits may also include suitable supports useful in performing the methods of the invention.

Peptides of the invention may be used to identify lead compounds for drug development. The structure of the peptides of the invention can be readily determined by a number of methods such as NMR and X-ray crystallography. A comparison of the structures of peptides similar in sequence, but differing in the biological activities they elicit in target molecules can provide information about the structure-activity relationship of the target. Information obtained from the examination of structure-activity relationships can be used to design either modified peptides, or other small molecules or lead compounds that can be tested for predicted properties as related to the target molecule. The activity of the lead compounds can be evaluated using assays similar to those described herein.

Information about structure-activity relationships may also be obtained from co-crystallization studies. In these studies, a peptide with a desired activity is crystallized in association with a target molecule, and the X-ray structure of the complex is determined. The structure can then be compared to the structure of the target molecule in its native state, and information from such a comparison may be used to design compounds expected to possess desired activities.

The invention features a method using a CPD motif, or a CPD motif binding partner, to design small molecule mimetics, agonists, or antagonists comprising determining the three dimensional structure of a CPD motif or CPD motif binding partner and providing a small molecule or peptide capable of binding to the CPD motif or CPD motif binding partner. Those skilled in the art will be able to produce small molecules or peptides that mimic the effect of the CPD motif or CPD motif binding partner and that are capable of easily entering the cell. Once a molecule is identified, the molecule can be assayed for its ability to bind a CPD motif or CPD motif binding partner, and the strength of the interaction may be optimized by making amino acid deletions, additions, or substitutions of by adding, deleting, or substituting a functional group. The additions, deletions, or modifications can be made at random or may be based on knowledge of the size, shape, and three-dimensional structure of the CPD motif or CPD motif binding partner.

Computer modelling techniques known in the art may also be used to observe the interaction of a CPD motif, CPD peptide, or peptide mimetic of the invention, and truncations and analogs thereof with an interacting molecule e.g. CPD motif binding partner, preferably an F-box Protein (for example, Homology Insight 11 and Discovery available from BioSym/Molecular Simulations, San Diego, Calif., U.S.A.). If computer modelling indicates a strong interaction, a CPD motif, CPD peptide, or peptide mimetic can be synthesized and tested for its ability to interfere with the binding of a motif, peptide, or mimetic with an interacting molecule.

Compositions and Treatments

A CPD motif, a molecule in a complex of the invention, a CPD motif binding partner, chimeric protein, antibody, complex, and CPD peptide of the invention, and agents, substances and compounds identified using the methods of the invention may be used to modulate ubiquitin dependent proteolysis, and they may be used to modulate signal transduction pathways which control cellular processes such as proliferation, growth, and/or differentiation of cells.

The disruption or promotion of the interaction between the molecules in complexes of the invention is also useful in therapeutic procedures. Therefore, the invention features a method for treating a subject having a condition characterized by an abnormality in a signal transduction pathway involving the interaction of a CPD motif and a CPD motif binding partner. The abnormality may be characterized by an abnormal level of interaction between the interacting molecules in a complex of the invention. An abnormality may be characterized by an excess amount, intensity, or duration of signal or a deficient amount, intensity, or duration of signal. An abnormality in signal transduction may be realized as an abnormality in cell function, viability, or differentiation state. The method involves disrupting or promoting the interaction (or signal) in vivo, or the activity of a complex of the invention. A compound that will be useful for treating a disease or condition characterized by an abnormality in a signal transduction pathway involving a complex of the invention can be identified by testing the ability of the compound to affect (i.e disrupt or promote) the interaction between the molecules in a complex. The compound may promote the interaction by increasing the production of a CPD motif containing protein, or by increasing expression of a CPD motif, or by promoting the interaction of the molecules in the complex. The compound may disrupt the interaction by reducing the production of a CPD motif containing protein, preventing expression of a CDP motif, or by specifically preventing interaction of the molecules in the complex.

A CPD motif, molecule, chimeric protein, CPD motif binding partner, antibody, or peptide of the invention, or agents, substances or compounds identified by a method of the invention may be used for the treatment of proliferative disorders including various forms of cancer such as leukemias, lymphomas (Hodgkins and non-Hodgkins), sarcomas, melanomas, adenomas, carcinomas of solid tissue, hypoxic tumors, squamous cell carcinomas of the mouth, throat, larynx, and lung, genitourinary cancers such as cervical and bladder cancer, breast, ovarian, colon, hematopoietic cancers, head and neck cancers, and nervous system cancers, benign lesions such as papillomas, arthrosclerosis, angiogenesis, and viral infections, in particular HIV infections, psoriasis, bone diseases, fibroproliferative disorders such as involving connective tissue, atherosclerosis and other smooth muscle proliferative disorders, chronic inflammation, and arthropathies such as arthritis. In addition to proliferative disorders, the treatment of differentiative disorders which result from, for example, de-differentiation of tissue which may be accompanied by abnormal reentry into mitosis. Such degenerative disorders that may be treated using the peptides and compositions of the invention include neurodegenerative disorders such as chronic neurodegenerative diseases of the nervous system, including Alzheimer's disease, Parkinson's disease, Huntington's chorea, amylotrophic lateral sclerosis and the like, as well as spinocerebellar degeneration.

A CPD motif, molecule, CPD peptide, CPD motif binding partner, antibody, substance, compound, agent, composition, and chimeric protein described herein can be administered to a subject either by themselves, or they can be formulated into pharmaceutical compositions for administration to subjects in a biologically compatible form suitable for administration in vivo. By “biologically compatible form suitable for administration in vivo” is meant a form of the substance to be administered in which any toxic effects are outweighed by the therapeutic effects.

The substances may be administered to living organisms including humans, and animals (e.g. dogs, cats, cows, sheep, horses, rabbits, and monkeys). Preferably the substances are administered to human and veterinary patients.

Administration of a “therapeutically active amount” is defined as an amount of a substance, at dosages and for periods of time necessary to achieve the desired result. For example, a therapeutically active amount of a substance may vary according to factors such as the disease state, age, sex, and weight of the individual, and the ability of the substance to elicit a desired response in the individual. Dosage regima may be adjusted to provide the optimum therapeutic response. For example, several divided doses may be administered daily or the dose may be proportionally reduced as indicated by the exigencies of the therapeutic situation. A therapeutically active amount can be estimated initially either in cell culture assays e.g. of neoplastic cells, or in animal models such as mice, rats, rabbits, dogs, or pigs. Animal models may be used to determine the appropriate concentration range and route of administration for administration to humans.

The active substance may be administered in a convenient manner by any of a number of routes including but not limited to oral, subcutaneous, intravenous, intraperitoneal, intranasal, enteral, topical, sublingual, intramuscular, intra-arterial, intramedullary, intrathecal, inhalation, transdermal, or rectal means. The active substance may also be administered to cells in ex vivo treatment protocols. Depending on the route of administration, the active substance may be coated in a material to protect the substance from the action of enzymes, acids and other natural conditions that may inactivate the substance.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the substances or compounds in association with one or more pharmaceutically acceptable vehicles or diluents, and contained in buffered solutions with a suitable pH and iso-osmotic with the physiological fluids.

A CPD motif, peptide, CPD motif binding partner, substance, compound, agent, antibody, or chimeric protein of the invention can be in a composition which aids in delivery into the cytosol of a cell. The substance may be conjugated with a carrier moiety such as a liposome that is capable of delivering the substance into the cytosol of a cell (See for example Amselem et al., Chem. Phys. Lipids 64:219-237, 1993 which is incorporated by reference). Alternatively, a substance may be modified to include specific transit peptides or fused to such transit peptides which are capable of delivering the substance into a cell. The substances can also be delivered directly into a cell by microinjection.

A CPD motif, molecule, peptide, CPD motif binding partner, substance, compound, agent, or chimeric protein of the invention may be therapeutically administered by implanting into a subject, vectors or cells capable of producing the CPD motif, molecule, peptide, CPD motif binding partner, agent, substance, or compound, or chimeric protein. In one approach cells that secrete a CPD motif, peptide, compound, substance, agent, or chimeric protein may be encapsulated into semipermeable membranes for implantation into a subject. The cells can be cells that have been engineered to express a CPD motif, molecule, peptide, agent, compound, substance, or chimeric protein. It is preferred that the cell be of human origin and the CPD motif, molecule, peptide, or chimeric protein be derived from a human CPD motif, molecule, peptide, or chimeric protein when the subject is a human.

A nucleic acid molecule encoding a CPD motif, peptide, CPD motif binding partner, compound, substance, agent, or chimeric protein of the invention may be used for therapeutic purposes. Viral gene delivery systems may be derived from retroviruses, adenoviruses, herpes or vaccinia viruses or from various bacterial plasmids for delivery of nucleic acid sequences to the target organ, tissue, or cells. Vectors that express the CPD motif, peptides, substances, compounds, agents, or chimeric proteins can be constructed using techniques well known to those skilled in the art (see for example, Sambrook et al.). Non-viral methods can also be used to cause expression of a CPD motif, peptide, compound, substance, agent, or chimeric protein of the invention in tissues or cells of a subject. Most non-viral methods of gene transfer rely on normal mechanisms used by mammalian cells for the uptake and transport of macromolecules. Examples of non-viral delivery methods include liposomal derived systems, poly-lysine conjugates, and artificial viral envelopes.

In viral delivery methods, vectors may be administered to a subject by injection, e.g. intravascularly or intramuscularly, by inhalation, or other parenteral modes. Non-viral delivery methods include administration of the nucleic acid molecules using complexes with liposomes or by injection; a catheter or biolistics may also be used.

The activity of a CPD motif, molecule, CPD motif binding partner, peptides, chimeric proteins, substances, compounds, agents, antibodies, and compositions of the invention may be confirmed in animal experimental model systems. The therapeutic efficacy and safety of a CPD motif, molecule, CPD motif binding partner, peptide, chimeric proteins, compounds, agents, substances, and compositions of the invention can be determined by standard pharmaceutical procedures in cell cultures or animal models. Therapeutic efficacy and toxicity may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The therapeutic index is the dose ratio of therapeutic to toxic effects and it can be expressed as the ED₅₀/LD₅₀ ratio. Pharmaceutical compositions which exhibit large therapeutic indices are preferred.

Antibodies that specifically bind the therapeutically active ingredient may be used to measure the amount of the therapeutic active ingredient in a sample taken from a patient for the purposes of monitoring the course of therapy.

The invention contemplates a method for evaluating a condition or disease of a patient suspected of exhibiting a condition or disease involving a CPD motif or complex of the invention. For example, biological samples from patients suspected of exhibiting a disease or condition may be assayed for the presence of CPD motifs or complexes of the invention. If a CPD motif or complex is normally present, and the development of the disease or condition is caused by an abnormal quantity of the CPD motif or complex, the assay should compare complex levels in the biological sample to the range expected in normal tissue of the same type.

Assays which may be undertaken include isolation of the CPD motif or complex, or assaying for the presence of a CPD motif or complex by exposing the sample to antibody specific for the CPD motif or complex, and detecting whether antibody has specifically bound. An assessment of the levels of a CPD motif or complex or nucleic acids encoding a CPD motif or a molecule of a complex of the invention in diseased tissue cells may provide valuable clues as to the course of action to be undertaken in treatment of the disease. Assays of this type are well known to those skilled in the art, and may include Northern blot analysis, RNAse protection assays, and PCR for determining nucleic acid levels. Assays for determining protein levels include Western blot analysis, immunoprecipitation, and ELISA analysis.

The invention also provides methods for studying the function of a CPD motif, or complex of the invention. Cells, tissues, and non-human animals lacking in the CPD motif, or complexes, or partially lacking in molecules in the complexes may be developed using recombinant expression vectors of the invention having specific deletion or insertion mutations in the molecules. A recombinant expression vector may be used to inactivate or alter the endogenous gene by homologous recombination, and thereby create CPD motif or complex deficient cells, tissues or animals. Null alleles may be generated in cells and may then be used to generate transgenic non-human animals.

The following non-limiting examples are illustrative of the present invention:

### EXAMPLE 1

Identification of a cdc4 WD40 Substrate Binding Motif

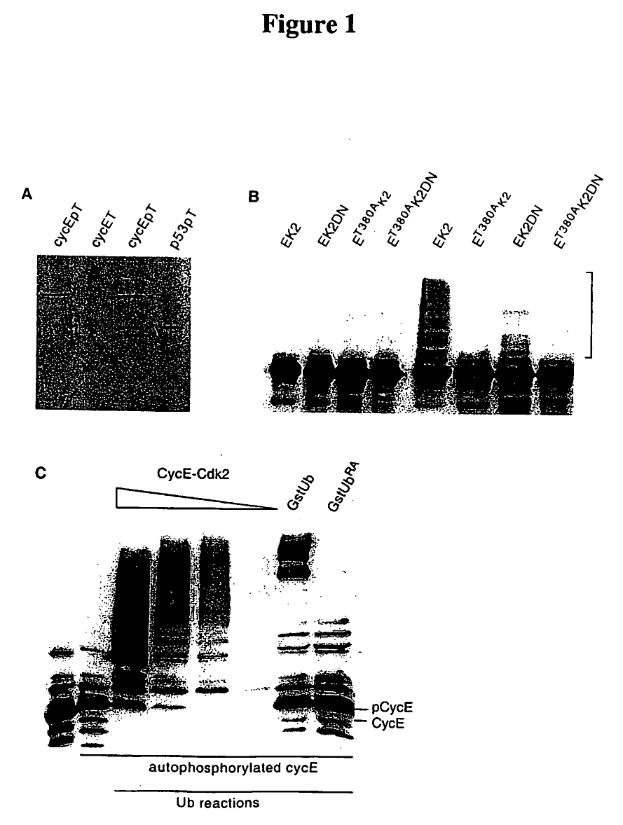

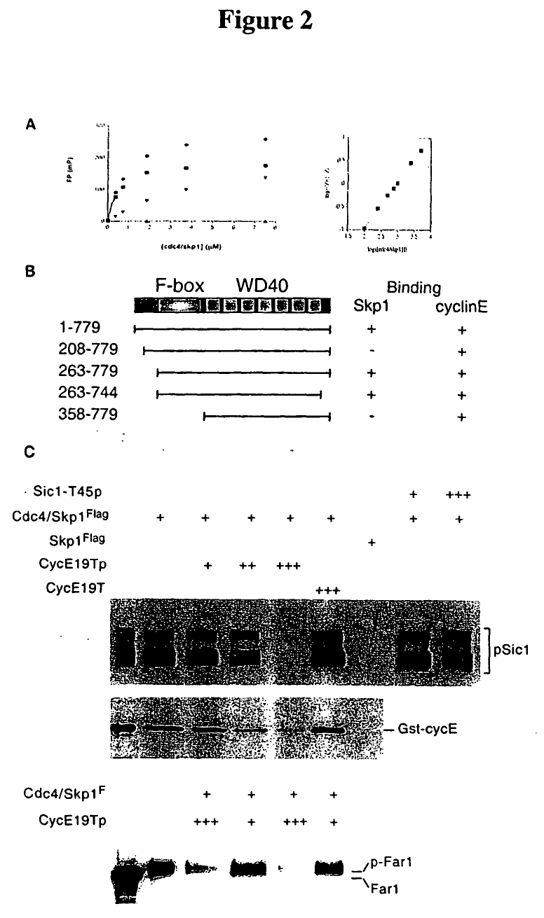

A mammalian cyclin E1 phosphopeptide (cycEpT19mer) corresponding to the region around Thr380 was found to bind to yeast Cdc4 and a biotinylated version of this peptide was able to precipitate cdc4 from baculovirus lysates expressing Cdc4. This interaction was found to be entirely dependent upon the phosphorylation of Thr380, as an unphosphorylated version of the peptide failed to interact with cdc4 (FIG. 1A). This interaction occurs with full-length cyclinE1, which was found to interact with the SCFcdc4 in vitro, and this interaction allows ubiquitination of cyclinE1. This interaction also requires phosphorylation of the cyclin E1 substrate, in this case by Cdc28-Clb2 kinase, and a phosphorylation site mutant (T380A) fails to be ubiquitinated by the SCFcdc4 complex (FIGS. 1A, B, and C). The cyclin E1 phosphopeptide then, serves as a reagent to probe cdc4 substrate binding. Equilibrium binding of Cdc4 to cyclin E pT380 peptide in solution was measured by fluorescence polarization. A dissociation constant Kd of 0.82 μM±0.08 μM was determined for the cycEpT19mer peptide binding the Cdc4 (FIGS. 1A, B, and C). A Hill plot of the data reveals a Hill coefficient of β=0.99, indicating that a single class of binding sites exists for this peptide on Cdc4 (FIG. 2A). Deletion analysis of Cdc4 suggests that the cyclin E1 binding site exists within the WD40 repeat region of Cdc4 (FIG. 2B). Deletion mutant cdc4AN failed to bind to Skp1, indicating that the F-box is non-functional, and yet it binds to cycE19P with affinity equivalent to the wild-type protein. Likewise, deletion of the region C-terminal to the WD40 domains does not alter cycE19P binding. A deletion of the F-box containing only the WD40 domains and C-terminal sequence retains binding to the cycEpT19mer. This serves to indicate that the WD40 domain repeat of cdc4 can act to bind peptides in a phosphorylation-dependent manner. Since WD40 repeats have not previously been shown to act in phosphorylation regulated interactions, this represents a novel paradigm. At least certain WD40 domains can thus be employed in protein-protein interactions regulated by S/T kinases, and join an emerging theme of pS/pT recognition modules that includes the FHA domain and certain WW domains.

The cyclin E1 binding site appears to be the same as that used by endogenous substrates of cdc4. Consistent with the Hill plot prediction of a single class of binding sites for cycE19P on cdc4, the cycE19P peptide was able to compete for the binding and ubiquitination of SCFcdc4 substrates in vitro. Ubiquitination of cyclinE1, Sic1, Far1 and Ash1 was blocked in the presence of excess cycE19P peptide (FIG. 2C). Thus it appears that Cdc4 contains a single substrate binding site within it's WD40 repeat region that recognizes phospho-peptide binding sites typified by the cycE19P peptide.

The PD-Box Targeting to SCFcdc4 is Phosphothreonine Dependent

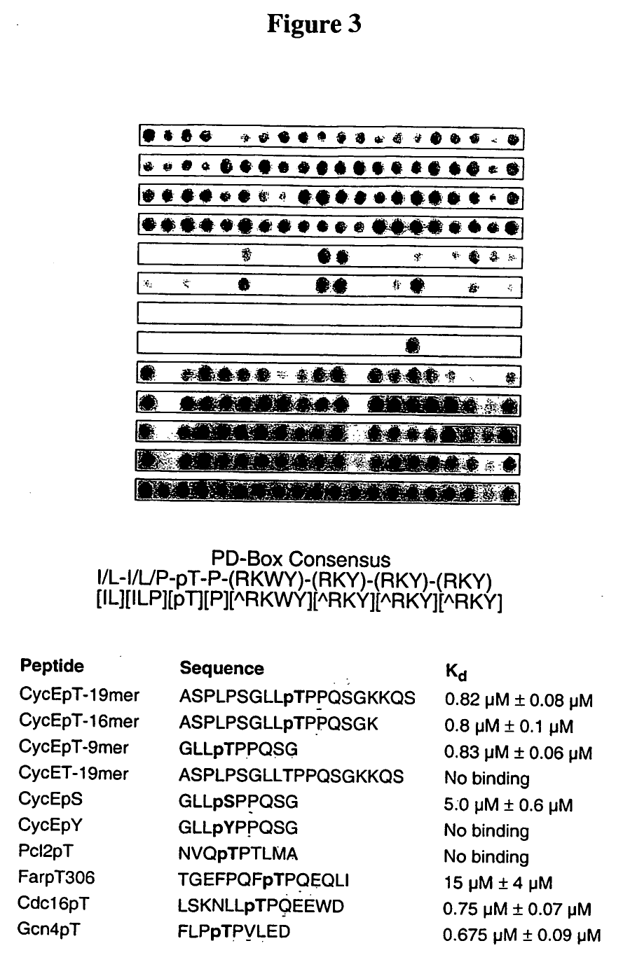

In order to ascertain the key peptide determinants for cdc4 recognition, a peptide SPOTS blot technique was employed. By varying each position of the cyclin E peptide to each of the 20 amino acids, a filter-based array was established to probe cdc4 binding specificity. Purified cdc4/skp1 complex binding to the solid-phase peptides was ascertained by probing for bound complex with antibody and subsequent chemiluminescent detection. The relative intensity of the spots within the array indicates the presence or absence of Cdc4/Skp1 binding (FIGS. 3A and 3B). The absolute requirement for a phosphothreonine residue is immediately apparent, and no other charged residue can act as a substitute. There is also an absolute requirement for proline at the +1 position, which is not surprising since this corresponds to the same absolute requirement for the relevant kinases that target substrates for degradation. The −1 position will accept only leucine, isoleucine, or proline, while the −2 position accepts only leucine or isoleucine. Positions beyond −2 do not appear to specify binding. At the +2 to +5 positions, most residues are tolerated, with the exception of basic or bulky hydrophobic amino acids (Lys, Arg, Tyr). The resulting consensus was termed a PD-box for Phosphorylation-dependent Degradation signal. A PD-box consensus was observed to be present in a number of other proteins, including Gcn4, Pcl7, and Cdc16. Gcn4 is a yeast transcriptional activator involved in regulating biosynthesis pathways that is known to be targeted for SCFcdc4 degradation by Pho85 phosphorylation on T165 (Meimoun MBC 2000). Not surprisingly, this site is part of a PD-box, and a peptide corresponding to the Gcn4 PD-box binds to cdc4 with high affinity (Kd=0.675 μM) (Table in FIG. 3). Pc17 is a member of a family of cyclin-like proteins that act as regulatory subunits for Pho85 kinase. Finally, Cdc16 is a component of the anaphase promoting complex (APC), and interacts with Cdc23p and Cdc27p. A peptide corresponding to the Cdc16 PD-box has a high affinity interaction with Cdc4, characterized by an equilibrium binding Kd=0.75 μM±0.07 μM. This is particularly interesting as it implies possible cross-talk between the SCF and APC complexes. Since Cdc4 has been found to play a role in G2/M transition and certain Cdc4 mutants can compensate for mutations in the APC component Cdc20 (Goh & Surana MCB 1999), this may indicate a regulated, direct interaction between these distinct E3 complexes.

The requirement for a phospho-threonine residue was further investigated using peptides corresponding to the cyclinE PD-box, but in which phospho-threonine was replaced by phospho-serine or phospho-tyrosine. The cycEpY peptide failed to bind, supporting the evidence that peptide recognition is not based merely upon the presence of a charge amino acid at the 0 position. By contrast, the cycEpS peptide was capable of interacting with cdc4, but with a with 6 fold lower affinity (Kd=6.0 μM±0.9 μM). Remarkably, the WD40 phospho-threonine recognition motif contained in cdc4 is capable of distinguishing threonine from serine. This suggests an additional level of complexity. While a given S/T kinase may readily phosphorylate both serine and threonine sites within a PD-box consensus motif, a WD40 binding partner such as cdc4 would be able to distinguish between the serine and threonine sites and bind with high affinity only to the phosphothreonine PD-box. PD-box sites containing serine at the 0 position would be sub-optimal and likely require multiple sites to allow efficient cdc4 binding.

The PD-Box Represents a Portable Tag for SCFcdc4 Ubiquitination/Degradation

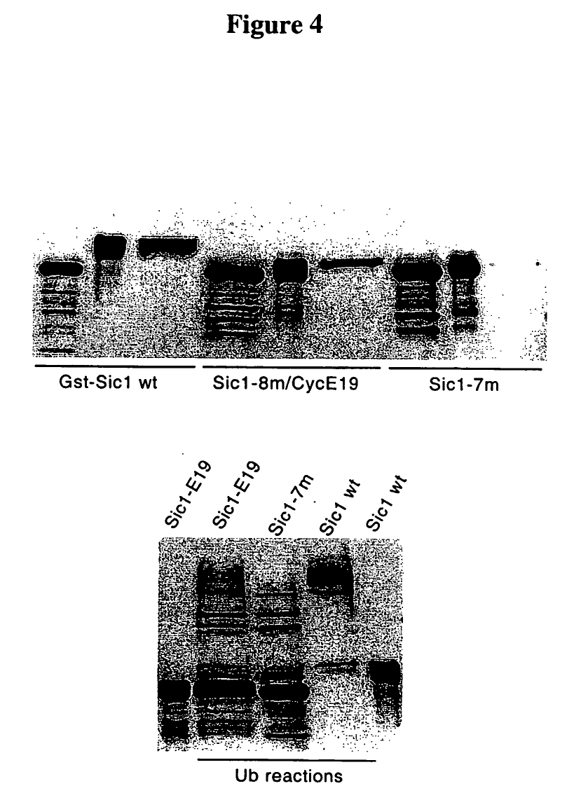

In order to test whether the PD-box of cyclin E1 could function to target protein degradation in the context of another protein, a chimeric Sic1 was constructed in which all endogenous Cdc4 recognition site had been abolished (Sic1-9mut) and into which the cyclin E1 peptide was inserted. In addition, it was observed that the nine sites shown by genetic evidence to be required for Cdc4 binding to Sic1 are all non-optimal PD-boxes in which either the +2 to +5 positions contain a basic residue, have threonine replaced with a lower affinity serine, or the −1 and −2 positions are sub-optimal. A version of sic1 was constructed in which the S69 and T4S sites were repaired to conform to the cyclinE1 PD-box consensus. Both the insertion of a cyclinE1 peptide into Sic1-8m, in which the 8 most important sites for Cdc4 interaction had been removed by mutation, served to allow mutant Sic1 interaction with, and ubiquitination by, SCFCdc4 in vitro (FIG. 4B).

The PD-box consensus does not permit basic residues at positions +2 to +5, which is in contrast to the specificity of Cdc28 kinase, suggesting that high affinity Cdc4 binding occurs on sites that are non-optimal for kinase recognition. Furthermore, certain SCFCdc4 substrates such as Sic1 contain multiple non-optimal PD-boxes indicating that there is a further degree of regulation.

### Example 2

Methods

Yeast Strains and Culture

Yeast strain construction, culture growth, FACS analysis and plasmid mutagenesis was performed as described (48). Strains, plasmids and oligonucleotides used are listed in Tables 2, 3 and 4. All mutated genes were sequenced in their entirety. A colony colour sectoring assay was used to monitor rates of chromosome loss as described (37). For Sic1 half-life experiments, cells bearing wild type and mutant alleles of SIC1^{HA} under control of the GAL1 promoter and integrated at the URA3 locus were arrested in G1 phase with α-factor, induced with galactose for 4 h shifted to repressive glucose medium and timepoints processed for immunoblot analysis with an anti-HA antibody as described (48). For expression of mutant SIC1 alleles at wild type levels, mutations were introduced into a plasmid based on MDM143 (14), in which the URA3 gene was inserted at a BglII site 769 nucleotides downstream from the SIC1 stop codon to create pMT2702. For each mutant, a SpeI to HpaI fragment encompassing nucleotides +65 to 792 of the SIC1 reading frame was cloned into pMT2702 and integrated at the chromosomal locus. The presence of mutant sequences was confirmed by synthetic restriction sites introduced with each mutation.

Recombinant Proteins, Binding Reactions and Kinase Assays

SCF complexes were purified from SF9 cells infected with recombinant baculoviruses and used in binding assays and ubiquitination reactions essentially as described (7). Gst-Skp1 expressed in BL21 codon plus cells (Stratagene) then purified on glutathione resin was used to capture full Cdc4 from insect cell lysates. Truncated forms of Cdc4 were enriched prior to Gst-Skp1 capture by affinity purification of hexahistidine fusion proteins on a metal chelate column. The Skp1-Cdc4 complexes were released from the Gst moiety by cleavage with TEV protease and further purified by size exclusion chromatography on a Superdex S75 or S200 column. Biotin labeled ASPLPSGLLpTPPQSGKKQS (SEQ ID NO. 1), ASPLPSGLLTPPQSGKKQS (SEQ ID NO. 12), and APPLSQEpTFSDLWK (SEQ ID NO. 13) were synthesized by addition of d-biotin (Sigma) with an Fmoc-e-aminocaproic acid (Bachem) spacer to carboxy-terminal peptides. Biotinylated peptides were purified by reverse-phase HPLC and confirmed by mass spectroscopy. Streptavidin-agarose beads (Sigma) were incubated in the presence of biotinylated peptide for 90 min. at 4° C. Beads were washed 3 times and then incubated with lysates from Cdc4 expressing baculovirus infected Sf9 cells. Beads were washed 4 times, after which SDS-PAGE gel loading buffer was added and the beads were boiled for 5 min. Proteins were separated by SDS-PAGE and visualized by silver stain. Peptide out-competition of phosphorylated substrates was carried out with immunopurified Skp1^{FLAG}-Cdc4 from insect cells, which were incubated with phosphopeptides prior to addition of phospho-Sic1 or phospho-Cyclin E-Cdk2. For kinase inhibition assays, purified recombinant Clb5-Cdc28 complex (30 ng) was incubated with mutant forms of Sic1 (40 ng) and histone H1 (HH1, 2 μg) for 1 h on ice prior to addition of [³²P]-γ-ATP and incubation at 25° C. for 30 min. Samples were separated by SDS-PAGE and visualized by autoradiography.

Peptide Synthesis

The peptides ASPLPSGLLpTPPQSGKKQS (SEQ ID NO. 1), ASPLPSGLLpTPPQSGK (SEQ ID NO. 2), GLLpTPPQSG (SEQ ID NO. 3), LLpTPP (SEQ ID NO. 14), GLLpSPPQSG (SEQ ID NO. 15) GLLpYPPQSG ((SEQ ID NO. 16), GLLTPPQSG (SEQ ID NO. 17), GKLpTPPQSG (SEQ ID NO. 18), GLKpTPPQSG (SEQ ID NO. 19), GLLpTAPQSG (SEQ ID NO. 20), GLLpTPKQSG (SEQ ID NO. 21), GLLpTPPKSG (SEQ ID NO. 22), GLLpTPPQKG (SEQ ID NO. 23), GLLpTPPQSK (SEQ ID NO. 24), GLLpTPPK(Ac)SG (SEQ ID NO. 25), FLPpTPVLED (SEQ ID NO 26), PKPLNLSKPIpSPPPSLKKTA (SEQ ID NO. 27), PPVpTPPMSP (SEQ ID NO. 28), VPVpTPSTTK (SEQ ID NO. 29), TGEFPQFpTPQEQLI (SEQ ID NO. 30), and VEQpTPKKPG (SEQ ID NO. 31) were synthesized as described previously (49).

Fluorescence Polarization Analysis

Equilibrium binding constant determination was carried out using fluorescence polarization on a Beacon 2000 Fluorescence Polarization System (Pan Vera, Wis.) equipped with a 100 μL sample chamber. Fluorescein-labeled probes were prepared through the reaction of carboxyterminal-peptides with 5-(and-6)-carboxyfluorescein succinimidyl ester (Molecular Probes), purified by reverse-phase HPLC, and confirmed by mass spectrometry. Binding studies were conducted with 5 nM fluorescein-labeled probe dissolved in PBS containing 100 μg/ml BSA and 1 mM dithiothreitol. Reaction mixtures were allowed to equilibrate for 10 minutes at room temperature prior to each measurement. All fluorescence polarization measurements were conducted at 22° C.

SPOTS Array Synthesis

Peptide arrays were constructed according to the spots-synthesis method (30). Acid-hardened cellulose membranes pre-derivatized with polyethylene glycol (AbiMed-Langfield, Germany) were spotted with a grid of Fmoc β-alanine (Bachem) prior to peptide synthesis. Standard Fmoc chemistry was used throughout (50). Fmoc protected and activated amino acids were spotted in high density 24×18 spot arrays on 130×90 mm membranes using an AbiMed ASP422 robot. All washing, Fmoc and side chain deprotection steps were done manually in polypropylene containers. The amino acids were at a concentration of 0.25M and were spotted at a volume of 0.2 μL, twice for each coupling reaction. Following peptide synthesis and side chain deprotection, membranes were blocked overnight in 5% skim milk. Purified Cdc4/Skp1 was added at 1 μM in TBS and incubated for 1 hour at 4° C. Membranes were wash d three times in TBS and incubated with anti-Skp1 polyclonal antiserum for 30 min, followed by anti-rabbit HRP secondary antibody (Sigma) in TBS. Detection was by SuperSignal enhanced chemiluminescence (Pierce).

Phosphorylation Requirements in Sic1 Recognition by Cdc4

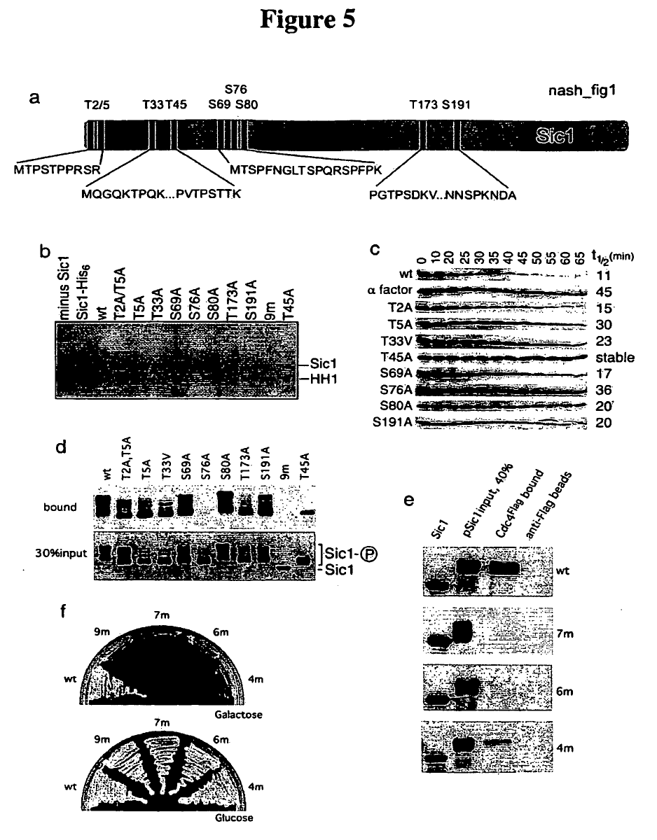

Previous analysis has shown that multiple phosphorylation sites contribute to Sic1 ubiquitination in vitro and degradation in vivo (13). To systematically determine the relative contributions of each of the 9 CDK consensus sites in Sic1, each individual site was mutated and the mutant proteins were assessed for stability in vivo, binding to Cdc4 and ubiquitination by SCF^{Cdc4} in vitro (FIG. 5). All of the mutant proteins inhibited Clb5-Cdc28 kinase activity to the same extent as wild type Sic1 (FIG. 5b). This was not unexpected since the region of Sic1 from residues 210-284, which is carboxyl-terminal to all potential CDK phosphorylation sites, exhibits full inhibitory activity in vivo (24,25). Note that the overall level of CDK-dependent phosphorylation of Sic1 was not significantly affected by mutation at individual phosphorylation sites but was completely abolished in a mutant lacking all nine CDK consensus sites (referred to as Sic1^{9m}). None of the individual mutants caused a G1 arrest when expressed at wild type levels, although the SIC1^{T45A} mutant did cause hyperpolarized bud growth in a fraction of the population, consistent with a delay in onset of Clb5-Cdc28 activity. In contrast, overexpression of the SIC1^{T45A} mutant or other mutants lacking multiple CDK phosphorylation sites from the GAL1 promoter permanently arrested cells in G1 phase (13,26). Repression of the various GAL1-SIC1 constructs in cells arrested at Start by mating pheromone allowed an estimate of Sic1 half-life in vivo (FIG. 5c). By this measure, Sic1T45A had a half-life of greater than 180 min, compared to a half-life of 13 min for wild type Sic1. Mutation of several other phosphorylation sites also had a detectable effect on Sic1 stability, consistent with the requirement for multiple phosphorylation events in Sic1 degradation. The rank order requirement for each site was T45, S76, T5, T33, followed by less significant contributions from other sites. Analysis of binding of the panel of Sic1 mutants to Cdc4 and the ability of the mutants to be ubiquitinated by SCF^{Cdc4} indicated that loss of either T45 or S76 sites severely compromises recognition of Sic1 by Cdc4 (FIG. 5d).

While it appears that many sites on Sic1 are necessary for degradation in vivo, it has not been determined which individual sites, or combinations thereof, are sufficient for degradation. Beginning with a fully mutant version of Sic1 that lacks all 9 CDK sites (Sic1^{9m}), increasing numbers of phosphorylation sites were restored and effects on viability and the Cdc4-Sic1 interaction were assessed. Surprisingly, serial re-introduction of the top ranked four sites failed to restore Sic1 binding to Cdc4 or degradation in vivo (FIG. 5e, f). Re-introduction of the top five ranked sites resulted in a modest Cdc4 binding, but was insufficient to restore degradation in vivo (FIGS. 5e, f). Thus, the phosphorylation dependent recognition mechanism involves an interaction between Cdc4 and most if not all CDK phosphorylation sites in Sic1.

Identification of a Phospho-Degron for Cdc4

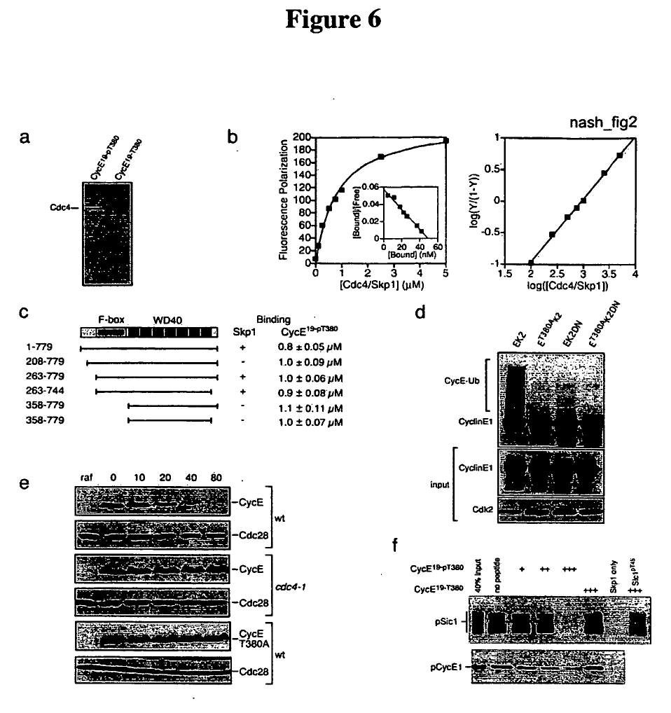

At least three possible modes of phospho-Sic1 binding to Cdc4 could be imagined: (i) a phosphorylation-dependent conformational change that exposes a cryptic binding epitope on Sic1; (ii) direct binding of multiple phosphorylated residues to multiple, distinct binding sites on Cdc4; (iii) equilibrium binding of multiple phosphorylated residues on Sic1 with a single high affinity recognition site on Cdc4. To investigate these different possibilities, the ability of various synthetic phosphopeptides to bind to Cdc4 was examined in vitro by fluorescence polarization and by their ability to capture Cdc4 from solution. Phosphopeptides corresponding to sequences centered on pT45 of Sic1 or another candidate interaction site in Far1 centered on pS87 (27), could neither bind stably to Cdc4, nor block the interaction between full length phosphorylated substrates and Cdc4 (Table 1). Peptides were next surveyed that correspond to other known sequences implicated in phosphorylation-dependent recognition by SCF complexes and a 19 residue phosphopeptide centered on T380 of mammalian cyclin E1 (CycE19-pT380) was discovered to bind to Cdc4 with high affinity. A biotinylated version of this phosphorylated peptide, but not an unphosphorylated peptide, was able to capture Cdc4 from crude lysates of insect cells infected with a recombinant baculovirus that expresses Cdc4 (FIG. 6a). Fluorescence polarization measurements performed with CycE19-pT380 and purified recombinant Cdc4 indicated a K_{d} of 1.0±0.05 μM and a Hill coefficient of 0.99 for the interaction, indicating a single class of high affinity binding site on Cdc4 (FIG. 6b). Deletion analysis of Cdc4 demonstrated that the CycE19-pT380 binding site is located within the WD40 repeat domain (FIG. 6c).

The pT380 site in cyclin E1 also functions within the context of the intact protein since full-length cyclin E1 could be bound and ubiquitinated by SCFCdc4 in vitro, in a phosphorylation dependent manner (FIG. 6d). Cyclin E1 degradation in yeast depends on Cdc4 function (FIG. 6e) and, as shown previously, on phosphorylation at T380 (28,29). Consistent with the Hill plot prediction of a single class of binding sites for CycE19-pT380 on Cdc4, the peptide was able to out-compete both binding and ubiquitination of cyclin E1, Sic1, and Far1 (FIG. 6f). The observation that the CycE19-pT380 peptide is able to out-compete cognate protein substrates of Cdc4 obviates the need to invoke the cryptic binding site model. Furthermore, since the Sic1 15-pT45 peptide does not measurably bind Cdc4 or compete with substrates, it is highly likely that all substrate interactions are dictated by the single high affinity site detected with the CycE19-pT380 peptide.

The Cdc4 Phospho-Degron Consensus Sequence

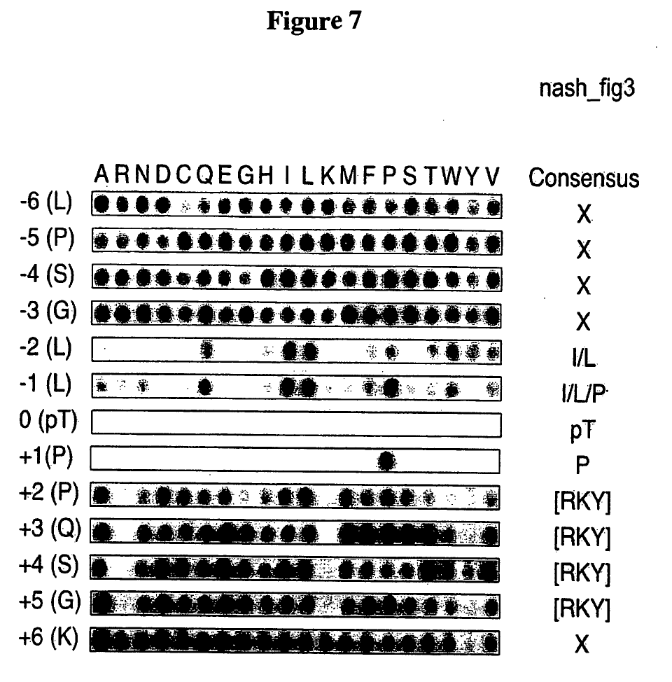

In order to identify the key peptide determinants for Cdc4 recognition, a peptide Spot blot technique was employed (30). By varying each position of the CycE19-pT380 peptide to each of the 20 natural amino acids, a filter-based array was constructed to probe Cdc4 binding specificity. Interaction of a purified Skp1-Cdc4 complex with peptides on the membrane was detected with an anti-Skp1 antibody (FIG. 7). Several characteristics of the binding site were revealed by the peptide Spots analysis. First, phosphorylation of the threonine residue and the presence of a proline residue at the +1 position are strictly required, consistent with the specificity of the cognate targeting CDK kinases. Second, binding specificity is contributed by sequences amino terminal to the phosphorylation site since there is a strong selection for leucine, isoleucine, or proline at the −1 position, while only leucine or isoleucine are accepted at the −2 position. Third, and quite unexpectedly, basic residues appear to be disfavored at the +2 to +5 positions, as is tyrosine. The optimal substrate selectivity of Cdc4 is therefore at odds with that of the cognate kinase Cdc28, which strongly prefers to phosphorylate S/T-P sequences followed by C-terminal basic residues (31).

A detailed quantitative analysis of the Cdc4 recognition motif was undertaken by fluorescence polarization measurements of defined variants of the CycE19-pT380 phosphopeptide (Table 1). The minimal peptide sequence required for binding was delimited to a core recognition sequence, LLpTPP, which bound Cdc4 with a K_{d} of 0.85±0.1 μM. As predicted by the peptide Spots analysis, introduction of basic residues in the +2 to +5 positions caused a decrease in binding affinity. Notably, the most severe decreases in binding occurred when a lysine was placed at the at the +2 and +3 positions in precise conformity to the optimal consensus sequence for CDK-directed phosphorylation. The detrimental effect of a positively charged residue at the +3 position was underscored by the finding that an acetylated lysine at this site did not diminish binding to Cdc4. The requirement for phosphorylation on threonine, as opposed to serine or tyrosine residues was also investigated. Not surprisingly, a phosphotyrosine-containing peptide (CycE19-pY380) failed to bind Cdc4. The equivalent phosphoserine peptide, CycE19-pS380, did interact with Cdc4, but with approximately 6-fold lower affinity (Table 1), indicating that the WD40 domain of Cdc4 partially discriminates phosphothreonine from phosphoserine, suggesting an additional level of complexity in substrate recognition. For brevity, the consensus binding sequence, L/1-L/1/P-pT-P<RKY>4 is referred to as the Cdc4 Phospho-Degron (CPD) motif, where <X> refers to disfavored residues.

CPD Motifs in Other Candidate Cdc4 Substrates.

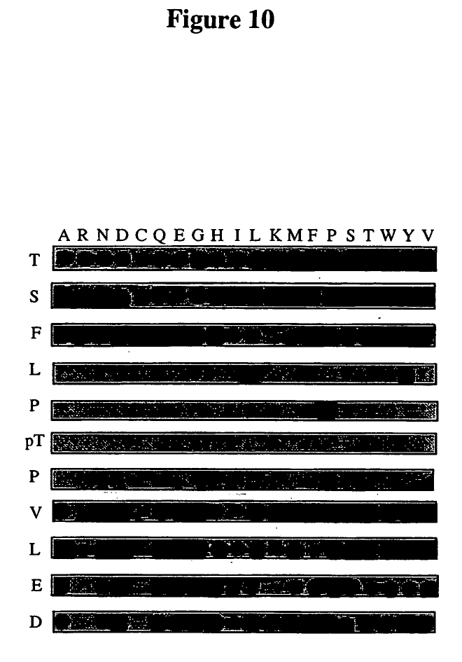

Database searches revealed that the CPD motif is present in many yeast proteins, including a recently characterized SCF^{Cdc4} substrate, the yeast transcriptional activator Gcn4 (32). The relevant targeting phosphorylation site on Gcn4, T165, is embedded in a CPD-like sequence (LPpTP) and indeed a phosphopeptide centered on Gcn4-pT165 bound to Cdc4 with a K_{d} of 0.88±0.1 μM (Table 1). FIG. 10 shows the SPOTS blot optimization of a CPD motif derived from a Gcn4 peptide.

Another known Cdc4 substrate, Far1, also contained two reasonable matches to the CPD motif, but not within regions previously implicated in Far1 stability (27,33). Sequences centered on T63 and T306 matched the CPD, and indeed a phosphopeptide corresponding to the region around T306 bound weakly to Cdc4 (Table 1). Phosphorylation of this site appears to contribute to activation of Far1 by the MAP kinase Fus3 (34), raising the possibility that Far1 activation is directly coupled to its recognition by SCF^{Cdc4}.

Several unanticipated candidate substrates emerged in database searches that, if phosphorylated, would be expected to bind to Cdc4. Intriguingly, a component of the APC/C, Cdc16, contained a CPD sequence that bound to Cdc4 with a K_{d}=0.9±0.1 μM (Table 1). This result implies possible regulation of the APC/C by SCF^{CdC4}, and may underlie the enigmatic role that Cdc4 appears to play in the G2/M transition (35). Despite the potential success of the database search, candidate substrates with more degenerate matches to the CPD, such as the Pho85 cyclin subunits Pc12 and Pc17 do not bind tightly to Cdc4 (Table 1). Numerous other candidate substrates appear to be ruled out based on non-overlapping subcellular localization with Cdc4, which is predominantly found in the nucleus (36).

The CPD is a Portable Degradation Signal

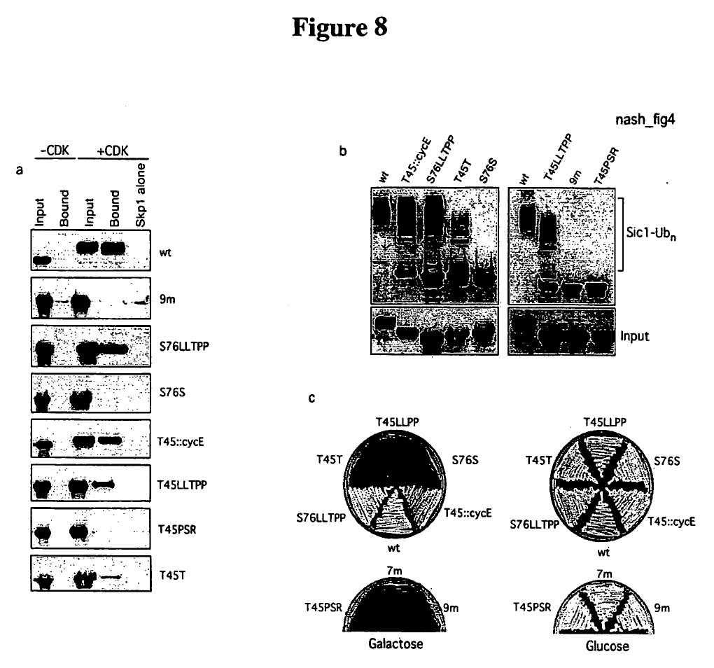

In order to test whether the CPD motif of cyclin E1 could target a heterologous protein for SCF^{Cdc4} dependent ubiquitination, the cyclin E1 peptide motif or derivatives thereof were inserted into the Sic1^{9m} variant that lacks endogenous phosphorylation sites. The full CycE19-pT380 sequence was placed at the T45 site of Sic19m (Sic19m-T45::CycE) where it was indeed able to confer both recognition and ubiquitination by SCFCdc4 in vitro (FIGS. 8a, b). Furthermore, the CycE19-pT380 sequence allowed elimination of Sic19m in vivo, as judged by cell viability when SIC19m-T45::CycE was overexpressed (FIG. 8c). To rule out the possibility that insertion of the full 19 residue cyclin E derived sequence might unduly contort Sic1 and allow recognition in an unnatural context, the core CPD motif was substituted around the T45 and S76 sites. Substitution of LLpTPP at the S76 site of Sic19m conferred effective phosphorylation-dependent ubiquitination in vitro and degradation in vivo, indicating that this single minimal motif is sufficient to confer Cdc4 recognition in the context of full-length Sic1 (FIGS. 8b, c). In contrast, substitution of LLpTPP at the T45 site allowed only poor recognition by Cdc4 and incomplete ubiquitination in vitro. Correspondingly, overexpression of SIC1^{9m-T45LLTPP} caused growth arrest. As suggested by the failure of some degenerate CPD peptides to bind Cdc4 (Table 1), additional local context effects may influence recognition of the CPD motif. As expected from the CPD consensus sequence, mutations that convert a single endogenous CDK site in Sic1 into an optimal CDK recognition sequence failed to allow Sic1 degradation in vivo (FIG. 8c).

Multiple Sub-Optimal CPD Motifs Set a Threshold for Sic1 Degradation

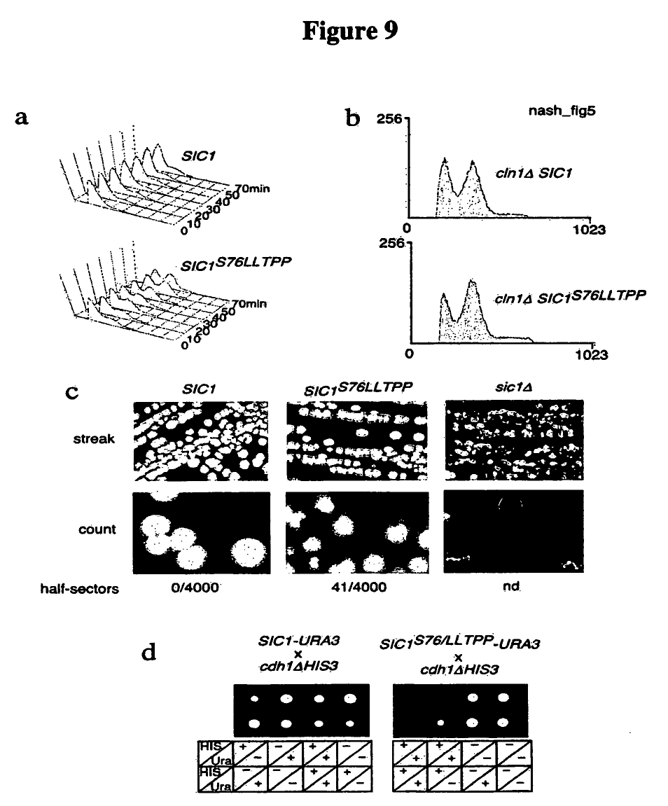

The importance of the presence of multiple weak CPD sites, as opposed to a single high affinity CPD motif, on the biological function of Sic1 in vivo was examined. To this end, a mutant version of Sic1 (Sic1^{7mS76LLTPP}) lacking seven of the endogenous CDK phosphorylation sites, but incorporating a single high affinity LLTPP motif was introduced in place of S76, at the chromosomal SIC1 locus under the control of the endogenous SIC1 promoter (endogenous T2/5 phosphorylation sites were not eliminated due to cloning constraints). Because Sic1 is needed to link DNA replication to other events at Start (10), the onset of DNA replication in wild type and SIC1^{7mS76LLTPP} strains was compared upon release from a mating pheromone-induced G1 phase arrest into sub-optimal nutrient conditions. Unlike wild type cells that delay all events at Start under these conditions, SIC1^{7mS76LLTPP} cells are unable to restrain DNA replication (FIG. 5a). Precocious DNA replication is also evident in rich media when Cln-Cdc28 activity is compromised, consistent with the notion that Sic1 degradation is determined by a balance of CDK activity and recognition by Cdc4 (FIG. 9b). A dire consequence of premature DNA replication in yeast and mammalian cells is genome instability (14,18), presumed to occur because of incomplete origin assembly in early G1 phase, which leads to incomplete genome replication in S phase. Genome stability was measured in wild type and Sic1 mutant strains by determining rates of chromosome loss in a sensitive colony sectoring assay (FIG. 9c). Based on this assay, it is estimated that the rate of chromosome loss is increased over 100-fold in the SIC¹^{7mS76LLTPP} strain compared to a wild type strain, an effect comparable to that observed for other mutants defective in chromosome transmission (37). The pivotal role of appropriately timed Sic1 elimination is illustrated by the rampant rate of chromosome loss in a sic1Δ strain (ref. 14; FIG. 9c). Sic1 also plays a crucial function at the end of mitosis, where it facilitates elimination of Clb-Cdc28 activity in order to re-establish G1 phase 8. Loss of Sic1 function in this context is manifest as sensitivity to perturbations in either the mitotic exit network or the APC/C activator Cdh1, both of which are necessary for cyclin destruction in late mitosis 38-40. To determine if an optimal CPD sequence compromises the mitotic exit function of Sic1, the progeny of a cross between SIC1^{7mS76LLTPP} and cdh1Δ double mutant strains were examined. As predicted, a single optimal CPD motif compromises Sic1 activity at the end of mitosis, as judged by the inability to recover SIC1^{7mS76LLTPP} cdh1Δ double mutants (FIG. 9d). This result also demonstrates that mitotic forms of CDK activity are competent to target Sic1 for degradation via the optimal CPD motif.

Single Optimal Versus Multiple Sub-Optimal CPD Motifs

The consensus binding site for the WD40 repeats of the F-box protein Cdc4 contains three main determinants: (i) an absolute requirement for phosphothreonine/serine followed by a proline residue; (ii) a strong preference for aliphatic leucine and isoleucine residues in the −2 and −1 positions; and (iii) a bias against basic residues in the +2 to +5 positions. Given the minimal experimentally determined CPD, LLpTPP, it is apparent why inspection of many known phosphorylation sites implicated in targeting various substrates to Cdc4 has failed to yield an obvious consensus sequence. Indeed, none of the phosphorylation sites necessary for degradation of Sic1 and Cdc6 conform to the ideal CPD consensus. For instance, the nine CDK sites in Sic1 are all non-optimal CPD motifs in that either a basic residue is present in the +2 to +5 positions, or a threonine phosphorylation site is replaced with a lower affinity serine site, or the −1 and −2 positions lack the preferred hydrophobic residues. Similarly, the eight CDK phosphorylation sites that influence Cdc6 recognition by Cdc4 lack one or more features of the ideal CPD (41-43). The apparent low affinity of each individual site in Sic1 for Cdc4 explains the requirement for multi-site phosphorylation. Stable binding of phospho-Sic1 to Cdc4 may therefore be achieved through a high local concentration of low affinity motifs, which drive equilibrium binding by increasing the overall avidity for a single high affinity site. Although other models could be considered, the presence of only a single class of high affinity binding site on Cdc4 for the CycE-pT380 phosphopeptide affords the simplest interpretation of the data. It appears that there is no absolute mechanistic requirement for multiple phosphorylation sites in substrate recognition by Cdc4, since Cdc4 is capable of efficiently capturing substrates that bear a single high affinity site, as in the case of Gcn4 (32), or when a single optimal CPD is introduced into a version of Sic1 that lacks all other phosphorylation sites.

Structural analysis indicates that WD40 repeat domains fold into a β-propeller structure (44). Interestingly, the N-terminal domain of clathrin forms a β-propeller that binds with low affinity to a single specific peptide motif from β-arrestin and the AP-3 adaptor complex via a peptide-in-groove interaction (45). The interaction of Cdc4 with the CPD might therefore resemble the association of clathrin with endocytic adaptors.

Other F-box proteins may bind their targets through the recognition of multiple low affinity sites. The LRR containing F-box protein Grr1 appears to target one of its substrates, Cln2, for degradation in a multi-site phosphorylation-dependent manner (4). Although phospho-degrons have been described for two other the F-box proteins, β-TrCP and Skp2, in neither case have the binding affinities been quantified nor have the motifs been optimized for binding. Intriguingly though, β-TrCP binds the sequence DSGΨXS in a fashion that requires phosphorylation on both serines (20), whereas Skp2 binds to a defined site on p27^{Kip1} with evidently weak affinity (21,22). Therefore, other phosphorylation sites may contribute to substrate recognition by these SCF complexes.

Discordance Between Kinase and Ubiquitin Ligase Substrate Recognition

Identification of the CPD sequence has uncovered an unexpected theme in phosphorylation-dependent protein recognition and degradation. That is, rather than a precise coincidence of kinase substrate specificity and modular phosphopeptide recognition, as commonly observed for the tyrosine kinases and SH2 domain binding (23), it appears that these two forces are partially at odds in SCFCdc4 substrate recognition. A dynamic balance between phosphorylation and recognition by the ubiquitination machinery could provide flexibility in substrate degradation to allow fine-tuning of irreversible regulatory switches, such as occur in cell cycle transitions. In principle, potential non-coincidence between kinase consensus sequences and phosphoprotein binding domains may have been unappreciated in other contexts, particularly in the numerous kinase-dependent signal transduction cascades that regulate cellular behavior in metazoans (23).

Multi-Site Phosphorylation and Biological Thresholds

In late G1 phase, it appears that a threshold level of Cln1/2-Cdc28 activity is required to activate events associated with Start, including elimination of Sic1 (10,11,13). If a single phosphorylation event were to determine the fate of Sic1, only a minimal level of G1 CDK activity would be required, a situation that would render the control of DNA replication susceptible to small fluctuations in CDK activity. Although a single, optimal CPD motif in Sic1 results in recognition by Cdc4 and consequent ubiquitination and degradation, it does not allow precise control of Start, resulting instead in precocious S-phase onset and chromosomal instability. That is, the single CPD motif fails to act as an appropriate biological switch for S-phase because it is recognized too efficiently. In contrast, degradation of wild-type Sic1 demands phosphorylation of at least 6 of 9 CDK sites, thereby imposing a much higher threshold of CDK activity. In one sense then, Sic1 acts as an integrator of CDK activity.

Multi-site phosphorylation is a common feature of many protein kinase substrates, and may promote regulation of events such as multi-site docking interactions, substrate dephosphorylation, subcellular localization, and protein activity (46). The requirement for multi-site phosphorylation that was observed for Sic1 within a cellular milieu in which kinases and phosphatases act in dynamic equilibrium can create an extraordinarily sharp biological switch (47). The targeting of Sic1 to Cdc4 by multiple sub-optimal phospho-degrons provides a model through which to understand how biological thresholds are set at the molecular level.

Having illustrated and described the principles of the invention in a preferred embodiment, it should be appreciated to those skilled in the art that the invention can be modified in arrangement and detail without departure from such principles. All modifications coming within the scope of the following claims are claimed.

All publications, patents and patent applications referred to herein are incorporated by reference in their entirety to the same extent as if each individual publication, patent or patent application was specifically and individually indicated to be incorporated by reference in its entirety.

TABLE 1
Measured affinities of peptides for Cdc4. Results are the average of at
least 3 individual sets of readings by fluorescence polarization. Values
for which saturation binding could not be achieved are indicated as
approximate (˜). ND indicates no binding detected by fluorescence
polarization up to [Cdc4/Skp1] of 10 μM. Errors are standard error
of the mean of all measurements (SEM).

| | | |
|---|---|---|
| Peptide Name | Peptide Sequence | K, (μM) |

Effect of peptide length

| | | |
|---|---|---|
| CycE-19mer | ASPLPSGLLpTPPQSGKKQS (SEQ ID | 1.0 ± 0.08 |
| | NO. 1) | |
| CycE-16mer | ASPLPSGLLpTPPQSGK (SEQ ID | 0.9 ± 0.1 |
| | NO. 2) | |
| CycE-9mer | GLLpTPPQSG (SEQ ID NO. 3) | 1.0 ± 0.05 |
| CycE-5mer | LLpTPP (SEQ ID NO. 48) | 0.85 ± 0.1 |
| Effect of pT | | |
| CycE-9mer | GLLpTPPQSG (SEQ ID NO. 3) | 1.0 ± 0.05 |
| CycEpS-9mer | GLLpSPPQSG (SEQ ID NO. 49) | 6.0 ± 0.9 |
| CycEpY-9mer | GLLpYPPQSG (SEQ ID NO. 50) | ND |
| CycEdeP-9mer | GLLTPPQSG (SEQ ID NO. 51) | ND |

Cost of basic residues

| | | |
|---|---|---|
| CycE-9mer | GLLpTPPQSG (SEQ ID NO. 3) | 1.0 ± 0.05 |
| CycEK₋₂-9mer | GKLpTPPQSG (SEQ ID NO. 52) | 12 ± 2 |
| CycEK₋₁-9mer | GLKpTPPQSG (SEQ ID NO. 53) | 6.0 ± 1.2 |
| CycEA₁-9mer | GLLpTAPQSG (SEQ ID NO. 54) | ND |
| CycEK₂-9mer | GLLpTPKQSG (SEQ ID NO. 55) | 6.3 ± 1.2 |
| CycEK₃-9mer | GLLpTPPKSG (SEQ ID NO. 56) | 5.1 ± 1.4 |
| CycEK₃Ac-9mer | GLLpTPPK(Ac)SG (SEQ ID NO. 57) | 1.0 ± 0.1 |
| CycEK₄-9mer | GLLpTPPQKG (SEQ ID NO. 58) | 4.3 ± 1.3 |
| CycEK₅-9mer | GLLpTPPQSK (SEQ ID NO. 59) | 2.4 ± 0.8 |

Potential cdc4 substrates

| | | |
|---|---|---|
| CycE-9mer | GLLpTPPQSG (SEQ ID NO.3) | 1.0 ± 0.1 |
| Gcn4-9mer | FLPpTPVLED (SEQ ID NO. 6) | 0.88 ± 0.1 |
| Far 1 pS₈₇ | PKPLNLSKPIpSPPPSLKKTA | ND |
| | (SEQ ID NO. 60) | |
| Ash 1 pT₂₉₀ | PPVpTPPMSP (SEQ ID NO. 61) | ˜25 ± 6 |
| SicpT₄₅ | VPVpTPSTTK (SEQ ID NO. 62) | ND |
| FarpT₃₀₆ | TGEFPQFpTPQEQLI (SEQ ID | ˜25 ± 6 |
| | NO. 63) | |
| p27pT₁₈₇ | VEQpTPKKPG (SEQ ID NO. 64) | ND |
| Cdc 16 | LSKNLLpTPQEEWD (SEQ ID | 0.9 ± 0.1 |
| | NO. 65) | |
| Pc17 | ELLpTPILAF (SEQ ID NO. 66) | ˜40 ± 11 |
| Pc12 | NVQpTPTLMA (SEQ ID NO. 67) | ND |

TABLE 2
List of plasmids employed in this study with relevant characteristics
and source.

| Plasmid | Relevant characteristic | Source |
|---|---|---|
| pMT1571 | PET16b-SIC1 | this study |
| pMT751 | PET16b-CDC34 | this study |
| pMT857 | PGAL1-SIC1 HIS3 CEN | this study |
| pMT2728 | PGAL1-SIC1^{9m} HIS3 CEN | this study |
| pMT2667 | PGAL1-SIC1^{7m} HIS3 CEN | this study |
| pMT2724 | PGAL1-SIC1^{T45LLTPP} HIS3 CEN | this study |
| pMT2732 | PGAL1-SIC1^{T45PSR} HIS3 CEN | this study |
| pMT2648 | pGAL1-SIC1^{T45::cycE}19 HIS3 CEN | this study |
| pMT2652 | PGAL1-SIC1^{S76LLTPP} HIS3 CEN | this study |
| pMT2656 | PGAL1-SIC1^{T45T} HIS3 CEN | this study |
| pMT2660 | PGAL1-SIC1^{S76S} HIS3 CEN | this study |
| pMT2668 | PGAL1-cycE1 HIS3 CEN | this study |
| pMT2669 | PGAL1-cycE1^{T380A} HIS3 CEN | this study |
| pMT919 | PGEX-3x-SIC1 | this study |
| pMT2726 | PGEX-3x-SIC1^{9m} | this study |
| pMT2666 | PGEX-3x-SIC1^{7m} | this study |
| pMT2722 | PGEX-3x-SIC1^{T45LLTPP} | this study |
| pMT2730 | PGEX-3x-SIC1^{T45PSR} | this study |
| pMT2646 | PGEX-3x-SIC1^{T45::cycE}19 | this study |
| pMT2650 | PGEX-3x-SIC1^{S76LLTPP} | this study |
| pMT2654 | PGEX-3x-SIC1^{T45T} | this study |
| pMT2658 | PGEX-3x-SIC1^{S76S} | this study |
| pMT2666 | PGEX-3x-SIC1^{7m} | this study |
| pMT2702 | SIC1-URA3 | this study |
| pMT2736 | SIC1^{S76LLTPP}-URA3 | this study |
| pMT2818 | Sic1ΔURA3 | this study |
| pMT2191 | PCS2-MT-cycE | J. Roberts |
| pMT2192 | PCS2-MT-cycE^{T380A} | J. Roberts |
| pMT2197 | PCMV-Cdk2 | E. Harlow |
| pMT2198 | PCMV-Cdk2DN | E. Harlow |

TABLE 3
List of yeast strains employed in the current study with relevant
genotype and source information.

| Strain | Relevant genotype | Source |
|---|---|---|
| KN699 | MATa, ade2-1, can1-100, his3-11, 15leu2-3, | K. Nasmyth |
| | 112, trp1-1, ura3, GAL1, psi+ | |
| MTY1996 | MATa, SIC1-URA3 | this study |
| MTY2052 | MATα, SIC1-URA3 | this study |
| MTY1998 | MATa, SIC1^{S76LLTPP}-URA3 | this study |
| MTY2060 | MATα, SIC1^{S76LLTPP}-URA3 | this study |
| MTY2067 | SIC-URA3, cln1ΔTRP1 | this study |
| MTY2069 | SIC1^{S76LLTPP}-URA3, cln1ΔTRP1 | this study |
| MTY2053 | MATa, SIC1-URA3, CFIII-HIS3-SUP11 | this study |
| MTY2054 | MATα, SIC1-URA3, CFIII-HIS3-SUP11 | this study |
| MTY2058 | MATa, SIC1^{S76LLTPP}-URA3, CFIII-HIS3- | this study |
| | SUP11 | |
| MTY2059 | MATα, SIC1^{S76LLTPP}-URA3, CFIII-HIS3- | this study |
| | SUP11 | |
| MTY2056 | MATa, sic1ΔURA3 | this study |
| MTY2057 | MATα, sic1ΔURA3 | this study |
| MTY2062 | MATa, sic1ΔURA3, CFIII-HIS3-SUP11 | this study |
| MTY2063 | MATα, sic1ΔURA3, CFIII-HIS3-SUP11 | this study |
| AA1120 | MATa, PDS1-HA-LEU2::pds1, cdh1::HIS3, | A. Amon |
| | ade2-1, leu2-3, ura3, trp1-1, his3-11, 15, | |
| | can1-100, GAL, psi+ | |

TABLE 4
Sequence of oligonucleotides used for mutagenesis of SIC1

| MTO | OLIGO | |
|---|---|---|
| # | NAME | SEQUENCE |
| 653 | T2A/T5A | c cgg atc cat atg gct cct agc gcg cca cca agg tcc aga |
| | | (SEQ ID NO. 68) |
| 654 | T33A | Atg caa ggt caa aag gcg ccc caa aag cct |
| | | (SEQ ID NO. 69) |
| 655 | S69A/S80A | Atg ggt atg acc gct cca ttt aat ggg ctt acg tct |
| | | Cct caa cgg gcc ccg ttt cca aaa tct (SEQ ID NO. 70) |
| 656 | T173A | tt aaa gat gta cct ggc gcc ccc agc gac aag |
| | | (SEQ ID NO. 71) |
| 657 | S191V | Aat tgg aac aac aac gtt ccg aaa aat gac |
| | | (SEQ ID NO. 72) |
| 795 | T45A | Cag aac cta gtc ccg cta gct ccc tca aca ac |
| | | (SEQ ID NO. 73) |
| 796 | S69A/S76A/ | Atg ggt atg acc gct cca ttt aat ggg ctt acg gct |
| | S80A | Cct caa cgg gcc ccg ttt cca aaa tct (SEQ ID NO. 74) |
| 797 | T45::cycE₁₉ | Ca cag aac cta gtc gct agc cct ctc ccc tca ggc |
| | | Ctc ctc acc ccg cca cag agc ggt aag aag cag |
| | | Agc aag tct ttt aaa aat gc (SEQ ID NO. 75) |
| 812 | S69A/ | Atg ggt atg acc gct cca ttt aat ggg ctt ctg act |
| | S76LLTPP/ | Cct cca ggg gcc ccg ttt cca aaa tct (SEQ ID NO. 76) |
| | S80A | |
| 813 | T45LLTPP | Cag aac cta gtc ctt ctc act ccc cca aca acc ggt |
| | | Tcc ttt aaa aat gcg (SEQ ID NO. 77) |
| 845 | T45PSR | Gtc act ccc tcg aga act aag tct (SEQ ID NO. 78) |

### REFERENCES

Yochem, J., and B. Byers. 1987. Structural comparison of the yeast cell division cycle gene CDC4 and a related pseudogene. J. Mol. Biol. 195: 233-245.

Zhang, H., R. Kobayashi, K. Galaktionov, and D. Beach. 1995. p19Skp1 and p45Skp2 are essential elements of the cyclin A-CDK2 S phase kinase. Cell 82: 915-925.

Dulic et al, Science, 1992, 25; 257(5078): 1958-61.

Koffet al, Science, 1992, 18; 257(5077): 1689-94.

Spruck, et al, Nature, 1999, 16; 401 (6750):297-300.

Winston et al, Curr Biol 1999,21; 9(20):1180-2.

Meimoun A, et al, 2000, Mol. Cell. Biol. 11 (3) 915-27.

Goh and Surana, Mol Cell Biol. 1999 19(8): 5512-22.

Cenciarelli, C. et al, Curr. Biol. 199, 21;9(20): 1177-9.

### REFERENCES (REFERENCED AS NUMBERS IN THE SPECIFICATION)

1. Hershko, A. & Ciechanover, A. The ubiquitin system. Annu Rev Biochem 67,425-79 (1998).

2. Varshavsky, A. Naming a targeting signal. Cell 64, 13-5 (1991).

3. Zachariae, W. & Nasmyth, K. Whose end is destruction: cell division and the anaphase-promoting complex. Genes Dev 13, 2039-58 (1999).

4. Patton, E. E., Willems, A. R. & Tyers, M. Combinatorial control in ubiquitin-dependent proteolysis: don't Skp the F-box hypothesis. Trends Genet 14, 236-43 (1998).

5. Koepp, D. M., Harper, J. W. & Elledge, S. J. How the cyclin became a cyclin: regulated proteolysis in the cell cycle. Cell 97, 4314 (1999).

6. Bai, C. et al. SKP1 connects cell cycle regulators to the ubiquitin proteolysis machinery through a novel motif, the F-box. Cell 86, 263-74 (1996).

7. Skowyra, D., Craig, K. L., Tyers, M., Elledge, S. J. & Harper, J. W. F-box proteins are receptors that recruit phosphorylated substrates to the SCF ubiquitin-ligase complex. Cell 91, 209-19 (1997).

8. Tyers, M. & Jorgensen, P. Proteolysis and the cell cycle: with this RING I do thee destroy. Curr Opin Genet Dev 10, 54-64 (2000).

9. Schwob, E., Bohm, T., Mendenhall, M. D. & Nasmyth, K. The B-type cyclin kinase inhibitor p40SIC1 controls the G1 to S transition in S. cerevisiae. Cell 79, 233-44 (1994).

10. Schneider, B. L., Yang, Q. H. & Futcher, A. B. Linkage of replication to Start by the Cdk inhibitor Sic1. Science 272, 560-2 (1996).

11. Tyers, M. The cyclin-dependent kinase inhibitor p40SIC1 imposes the requirement for CLN G1 cyclin function at Start. Proc. Natl. Acad. Sci. USA 93, 7772-7776 (1996).

12. Feldman, R. M., Correll, C. C., Kaplan, K. B. & Deshaies, R. J. A complex of Cdc4p, Skp1p, and Cdc53p/cullin catalyzes ubiquitination of the phosphorylated CDK inhibitor Siclp. Cell 91, 221-30 (1997).

13. Verma, R. et al. Phosphorylation of Sic1p by G1 Cdk required for its degradation and entry into S phase. Science 278, 455460 (1997).

14. Nugroho, T. T. & Mendenhall, M. D. An inhibitor of yeast cyclin-dependent protein kinase plays an important role in ensuring the genomic integrity of daughter cells. Mot. Cell. Biol. 14, 3320-3328 (1994).

15. Amati, B. & Vlach, J. Kipl meets SKP2: new links in cell-cycle control. Nat Cell Biol 1, E91-3 (1999).

16. Winston, J. T., Chu, C. & Harper, J. W. Culprits in the degradation of cyclin E apprehended. Genes Dev 13, 2751-7 (1999).

17. Sheaff, R. J., Groudine, M., Gordon, M., Roberts, J. M. & Clurman, B. E. Cyclin E-CDK2 is a regulator of p27Kip1. Genes Dev 11, 1464-1478 (1997).

18. Spruck, C. H., Won, K. A. & Reed, S. I. Deregulated cyclin E induces chromosome instability. Nature 401, 297-300 (1999).

19. Maniatis, T. A ubiquitin ligase complex essential for the NF-kB, Wnt/Wingless, and hedgehog signaling pathways. Genes Dev 13, 505-510 (1999).

20. Yaron, A. et al. Identification of the receptor component of the IkBa-ubiquitin ligase. Nature 396, 590-594 (1998).

21. Carrano, A. C., Eytan, E.; Hershko, A. & Pagano, M. SKP2 is required for ubiquitin-mediated degradation of the CDK inhibitor p27. Nat Cell Biol 1, 193-9 (1999).

22. Tsvetkov, L. M., Yeh, K. H., Lee, S. J., Sun, H. & Zhang, H. p27Kip1 ubiquitination and degradation is regulated by the SCFSkp2 complex through phosphorylated Thr187 in p27. Curr Biol 9, 661-4 (1999).

23. Pawson, T. & Nash, P. Protein-protein interactions define specificity in signal transduction. Genes Dev. 14, 102747(2000).

24. Verma, R., Feldman, R. M. & Deshaies, R. J. SIC1 is ubiquitinated in vitro by a pathway that requires CDC4, CDC34, and cyclin/CDK activities. Mol Biol Cell 8, 1427-37 (1997).

25. Hodge, A. & Mendenhall, M. D. The cyclin-dependent kinase inhibitory domain of the yeast Sic1 protein is contained within the C-terminal 70 amino acids. Mol. Gen. Genet. 262, 55-64 (1999).

26. Schneider, B. L. et al. Yeast G1 cyclins are unstable in G1 phase. Nature 395, 86-9 (1998).

27. Henchoz, S. et al. Phosphorylation- and ubiquitin-dependent degradation of the cyclin-dependent kinase inhibitor Far1p in budding yeast. Genes Dev 11, 3046-60 (1997).

28. Clurman, B. E., Sheaff, R. J., Thress, K., Groudine, M. & Roberts, J. M. Turnover of cyclin E by the ubiquitin-proteasome pathway is regulated by cdk2 binding and cyclin phosphorylation. Genes Dev 10, 1979-90 (1996).

29. Won, K. A. & Reed, S. 1. Activation of cyclin E/CDK2 is coupled to site-specific autophosphorylation and ubiquitin-dependent degradation of cyclin E. EMBO J. 15,4182-4193 (1996).

30. Frank, R. Spot-synthesis: an easy technique for positionally addressable, parallel chemical synthesis on a membrane support. Tetrahedron 48, 9217-9232 (1992).

31. Songyang, Z. et al. Use of an oriented peptide library to determine the optimal substrates of protein kinases. Curr Biol 4, 973-82 (1994).

32. Meimoun, A. et al. Degradation of the transcription factor Gcn4 requires the kinase Pho85 and the SCFCDC4 ubiquitin-ligase complex. Mol Biol Cell 11, 915-27 (2000).

33. McKinney, J. D. & Cross, F. R. FAR1 and the G1 phase specificity of cell cycle arrest by mating factor in Saccharomyces cerevisiae. Mol. Cell. Biol. 15, 2509-2516 (1995).

34. Gartner, A. et al. Pheromone-dependent G1 cell cycle arrest requires Far1 phosphorylation, but may not involve inhibition of Cdc28-Cln2 kinase, in vivo. Mol Cell Biol 18, 3681-91 (1998).

35. Goh, P. Y. & Surana, U. Cdc4, a protein required for the onset of S phase, serves an essential function during G2/M transition in Saccharomyces cerevisiae. Mol Cell Biol 19, 5512-22 (1999).

36. Blondel, M. et al. Nuclear-specific degradation of Far] is controlled by the localization of the F-box protein Cdc4. EMBO J. 19, 6085-97 (2000).

37. Spencer, F., Gerring, S. L., Connelly, C. & Hieter, P. Mitotic chromosome transmission fidelity mutants in Saccharomyces cerevisiae. Genetics 124,237-49 (1990).

38. Schwab, M., Lutum, A. S. & Seufert, W. Yeast Hctl is a regulator of Clb2 cyclin proteolysis. Cell 90, 683-93 (1997).

39. Visintin, R., Prinz, S. & A., A. Cdc20 and Cdh1, a familiy of substrate-specific activators of APC-dependent proteolysis. Science 278, 460-463 (1997).

40. Visintin, R. et al. The phosphatase Cdc14 triggers mitotic exit by reversal of Cdk-dependent phosphorylation. Mol Cell 2, 709-18 (1998).

41. Drury, L. S., Perkins, G. & Diffley, J. F. The Cdc4/34/53 pathway targets Cdc6p for proteolysis in budding yeast. EMBO J. 16, 5966-76 (1997).

42. Elsasser, S., Chi, Y., Yang, P. & Campbell, J. L. Phosphorylation controls timing of Cdc6p destruction: A biochemical analysis. Mol Biol Cell 10, 3263-77 (1999).

43. Drury, L. S., Perkins, G. & Diffley, J. F. The cyclin-dependent kinase Cdc28p regulates distinct modes of Cdc6p proteolysis during the budding yeast cell cycle. Curr Biol 10, 23140 (2000).

44. Fulop, V. & Jones, D. T. Beta propellers: structural rigidity and functional diversity. Curr Opin Struct Biol 9, 715-21 (1999).

45. ter Haar, E., Harrison, S. C. & Kirchhausen, T. Peptide-in-groove interactions link target proteins to the beta-propeller of clathrin. Proc Natl Acad Sci USA 97, 1096-100 (2000).

46. Cohen, P. The regulation of protein function by multisite phosphorylation: a 25 year update. Trends Biochem. Sci. (in press)

47. Ferrell, J. E. Tripping the switch fantastic: how a protein kinase cascade can convert graded inputs into switch-like outputs. Trends Biochem. Sci. 21, 460-466 (1996).

48. Willems, A. R. et al. Cdc53 targets phosphorylated G1 cyclins for degradation by the ubiquitin proteolytic pathway. Cell 86, 453-463 (1996).

49. van der Geer, P., Wiley, S., Gish, G. D. & Pawson, T. The Shc adaptor protein is highly phosphorylated at conserved, twin tyrosine residues (Y239/240) that mediate protein-protein interactions. Curr Biol 6, 1435-44 (1996).

50. Fields, G. B. & Noble, R. L. Solid phase peptide synthesis utilizing fluorenylmethoxycarbonyl amino acids. Int. J. Pept. Protein Res 35, 161-214 (1990).

We claim:

1. An isolated Cdc4 Phospho-Degron motif (“CPD motif”) that targets molecules for ubiquitin-dependent proteolysis.

2. A CPD motif as claimed in claim 1 comprising consensus sequence X²-X³-pThr-Pro-X4 or X²-X³-pThr-Pro-X⁴-X⁵-X⁶-X⁷ wherein X² represents Leu, Pro, or Ile, preferably Leu or Ile; X³ represents Leu, Ile, Val, or Pro, preferably Ile, Leu, or Pro; X⁴, X⁵ and X⁶ represent any amino acid except basic and bulky hydrophobic amino acids, preferably X⁴ is any amino acid except Arg, Lys, Tyr, or Trp, more preferably X⁴ is Ile, Val, Pro, or Gin, preferably X⁵ and X⁶ are any amino acid except Arg, Lys, or Tyr and more preferably X⁵ is Gin, Leu, Met, Thr, or Glu, and X⁶ is Gin, Ala, Thr, Glu, or Ser; X⁷ is any amino acid, preferably not a basic or bulky hydrophobic amino acid, more preferably X⁷ is any amino acid except Arg, Lys, or Tyr, most preferably X⁷ is Leu, Trp, Asp, Pro, or Gly.

3. A CPD motif as claimed in claim 1 comprising consensus sequence X¹-Leu/Gly/Tyr-Pro-pThr-Pro-X⁹wherein X¹ represents 0 to 100 amino acids, preferably 0 to 50, more preferably 0 to 20, most preferably 0 to 10 amino acids, and X⁹ represents 0 to 100 amino acids, preferably 0 to 50, more preferably 0 to 20, most preferably 0 to 10 amino acids, or represents X¹⁰-X¹¹-X¹²-X¹³-X¹⁴ wherein X¹⁰ ia any amino acid except Arg, X¹¹ is any amino acid except Cys, X¹² is any amino acid except Arg, Cys, and Lys, X¹³ is any amino acid except Arg and Cys, and X¹⁴ represents 0 to 100 amino acids, preferably 0 to 50, more preferably 0 to 20, most preferably 0 to 10 amino acids.

4. A cyclinE1, Gcn4, Far1, Ash1, Sic1, Cdcl6, or Pc17 CPD motif.

5. A molecule derived from a CPD motif as claimed in claim 1, 2, or 3.

6. A peptide of the formula:
X¹-X²-X³-pThr-Pro-X⁴-X⁸
wherein X¹ represents 0 to 100 amino acids, preferably 0 to 50, more preferably 0 to 20, most preferably 0 to 10 amino acids, X² represents Leu, Pro, or Ile, preferably Leu or Ile; X³ represents Leu, Ile, Val, or Pro, preferably Ile, Leu, or Pro; X⁴ represents any amino acid except basic and bulky hydrophobic amino acids, preferably X⁴ is any amino acid except Arg, Lys, or Tyr more preferably X⁴ is Ile, Val, Pro, or Gin, and X⁸ represents 0 to 100 amino acids, preferably 0 to 50, more preferably 0 to 20, most preferably 0 to 10 amino acids.

7. A peptide of the formula:
X¹-X²-X³-pThr-Pro-X4-X⁵-X⁶-X⁷-X⁸
wherein X¹ represents 0 to 100 amino acids, preferably 0 to 50, more preferably 0 to 20, most preferably 0 to 10 amino acids; X² represents Leu, Pro, or Ile, preferably Leu or Ile; X³ represents Leu, Ile, Val, or Pro, preferably Tie, Leu, or Pro; X⁴, X⁵ and X⁶ represent any amino acid except basic and bulky hydrophobic amino acids, preferably X⁴ is any amino acid except Arg, Lys, Tyr, or Trp, more preferably X⁴ is Ile, Val, Pro, or Gin, preferably X⁵ and X⁶ are any amino acid except Arg, Lys, or Tyr and more preferably X⁵ is Gin, Leu, Met, Thr, or Glu, and X⁶ is Gin, Ala, Thr, Glu, or Ser; X⁷ is any amino acid, preferably not a basic or bulky hydrophobic amino acid, more preferably X⁷ is any amino acid except Arg, Lys, or Tyr, most preferably X⁷ is Leu, Trp, Asp, Pro, or Gly; and X⁸ represents 0 to 100 amino acids, preferably 0 to 50, more preferably 0 to 20, most preferably 0 to 10 amino acids.

8. A peptide as claimed in claim 6 or 7 which binds to Cdc4 with a K_{d} less than 25 μM and which is capable of mediating ubiquitin-dependent proteolysis.

9. A peptide of the formula:
X¹-Leu/Gly/Tyr-Pro-pThr-Pro-X⁹
wherein X¹ represents 0 to 100 amino acids, preferably 0 to 50, more preferably 0 to 20, most preferably 0 to 10 amino acids, and X⁹ represents 0 to 100 amino acids, preferably 0 to 50, more preferably 0 to 20, most preferably 0 to 10 amino acids, or represents X¹⁰-X¹¹-X¹²-X¹³-X¹⁴ wherein X¹⁰ is any amino acid except Arg, X¹¹ is any amino acid except Cys, X¹² is any amino acid except Arg, Cys, and Lys, X¹³ is any amino acid except Arg and Cys, and X¹⁴ represents 0 to 100 amino acids, preferably 0 to 50, more preferably 0 to 20, most preferably 0 to 10 amino acids.

10. A peptide according to any preceding claim or a molecule derived therefrom that interacts with or alters the function of a SCF complex.

11. A chimeric protein comprising at least one CPD motif as claimed in any preceding claim.

12. A chimeric protein comprising a peptide according to any preceding claim fused to a target protein and/or a targeting domain that is capable of directing the protein to a desired cellular component or specific cell type or tissue.

13. A nucleic acid molecule that encodes a CPD motif as claimed in claim 1, 2, 3, or 4.

14. A nucleic acid molecule that encodes a peptide as claimed in any preceding claim.

15. A vector comprising a nucleic acid molecule as claimed in claim 13 or 14.

16. A host cell containing a nucleic acid molecule as claimed in claim 13 or 14.

17. An antibody specific for a CPD motif as claimed in claim 1, 2, 3, or 4 or a peptide as claimed in any preceding claim.

18. A method for identifying a substance which interacts with a CPD motif as claimed in claim 1, 2, 3, or 4 comprising (a) reacting the CPD motif with at least one test substance which potentially can interact with the CPD motif under conditions which permit the formation of complexes between the substance and CPD motif, and (b) detecting binding, wherein detection of binding indicates the substance interacts with the CPD motif.

19. A method as claimed in claim 18 wherein binding is detected by assaying for complexes, for free substance, for non-complexed CPD motif, or for activation of the CPD motif.

20. A substance identified using a method as claimed in claim 18 or 19.

21. A complex comprising a CPD motif as claimed in claim 1, 2, 3, or 4 and a CPD motif binding partner.

22. A method for evaluating a compound for its ability to modulate ubiquitin-dependent proteolysis through a CPD motif as claimed in claim 1, 2, 3, or 4 comprising providing a CPD motif, a CPD motif binding partner, and a test compound under conditions which permit the formation of complexes between the CPD motif and CPD motif binding partner, and removing and/or detecting complexes.

23. A method as claimed in claim 22 wherein the CPD motif binding partner is an F-box Protein.

24. A method as claimed in claim 23 wherein the CPD motif binding partner is a WD40-repeat protein.

25. A method for identifying agents to be tested for an ability to modulate a signal transduction pathway by testing for their ability to affect the interaction between a CPD motif and a CPD motif binding partner, wherein a complex formed by such interaction is part of the signal transduction pathway.

26. A method as claimed in claim 25 wherein the compound promotes the interaction between the CPD motif and CPD motif binding partner by increasing production of a CPD motif, increasing expression of a CPD motif, or by promoting the interaction.

27. A method as claimed in claim 25 wherein the compound disrupts the interaction between the CPD motif and CPD motif binding partner by preventing expression of the CPD motif, or by preventing or interfering with the interaction.

28. A method for identifying an agent to be tested for its ability to modulate ubiquitin-dependent proteolysis of a regulatory protein involving interaction of multiple low affinity binding sites on the protein with an F-box protein comprising:
(a) selecting a sequence motif of a low affinity binding site;
(b) optimizing the sequence motif so that a peptide comprising the sequence motif or a peptide mimetic thereof is capable of interacting with the F-box protein with high affinity; and
(c) synthesizing an agent comprising a peptide comprising the optimized sequence motif or peptide mimetic thereof;
(d) optionally testing the agent in in vivo or in vitro assays to ascertain if the agent modulates ubiquitin-dependent proteolysis of the F-box protein.

29. A method according to claim 28 further comprising preparing a quantity of the agent.

30. An agent identified by a method of claim 28.

31. A method for selectively degrading a target protein in a cell by ubiquitin-dependent proteolysis comprising administering to the cell a CPD motif as claimed in claim 1, 2, 3, or 4, or a peptide or chimeric protein as claimed in any preceding claim in an amount effective to selectively degrade the target protein in the cell.

32. A method of treating a disease or condition where affected cells have a defective target protein comprising administering an effective amount of a CPD motif as claimed in claim 1, 2, 3, or 4 to promote degradation of the target protein in cells by ubiquitin-dependent proteolysis.

33. A method as claimed in claim 32 wherein the CPD motif is administered by introducing into the cells a nucleic acid molecule encoding the CPD motif.

34. A method as claimed in claim 32, wherein the target protein is a mutated target protein or over expressed target protein.

35. A composition comprising a CPD motif, a peptide, or an agent as claimed in any preceding claim, and a pharmaceutically acceptable carrier, excipient or diluent.

36. A method for modulating proliferation, growth, and/or differentiation of cells comprising introducing into the cells a CPD motif, a peptide, or a composition as claimed in any preceding claim.

37. Use of a CPD motif as claimed in claim 1, 2, 3, or 4 or a peptide as claimed in any preceding claim to modulate ubiquitin dependent proteolysis.

38. Use of a CPD motif as claimed in claim 1, 2, 3, or 4 or a peptide as claimed in any preceding claim to modulate cell proliferation, growth, and/or differentiation in cells.

39. Use of a CPD motif as claimed in claim 1, 2, 3, or 4, or a peptide or agent as claimed in any preceding claim to manufacture a medicament to modulate proliferation, growth, and/or differentiation of cells. A method of conducting a drug discovery business comprising:
(a) providing one or more assay systems for identifying agents by their ability to inhibit or potentiate the interaction of a regulatory protein and an F-box protein;
(b) conducting therapeutic profiling of agents identified in step (a), or further analogs thereof, for efficacy and toxicity in animals; and
(c) formulating a pharmaceutical preparation including one or more agents identified in step (b) as having an acceptable therapeutic profile.

40. The method of claim 39, including a step of establishing a distribution system for distributing the pharmaceutical preparation for sale

41. The method of claim 39, including establishing a sales group for marketing the pharmaceutical preparation.

42. A method of conducting a target discovery business comprising:
(a) providing one or more assay systems for identifying agents by their ability to inhibit or potentiate the interaction of a regulatory protein and an F-box protein;
(b) (optionally) conducting therapeutic profiling of agents identified in step (a) for efficacy and toxicity in animals; and
(c) licensing, to a third party, the rights for further drug development and/or sales for agents identified in step (a), or analogs thereof.


SEQUENCE LISTING (78 sequences)

SEQ ID NO: 1 - protein, 19 aa, Artificial Sequence
  CPD Peptide
ASPLPSGLLTPPQSGKKQS

SEQ ID NO: 2 - protein, 16 aa, Artificial Sequence
  CDP Peptide
ASPLPSGLLTPPQSGK

SEQ ID NO: 3 - protein, 9 aa, Artificial Sequence
  CDP Peptide
GLLTPPQSG

SEQ ID NO: 4 - protein, 14 aa, Artificial Sequence
  CDP Peptide
TGEFPQFTPQEQLI

SEQ ID NO: 5 - protein, 13 aa, Artificial Sequence
  CDP Peptide
LSKNLLTPQEEWD

SEQ ID NO: 6 - protein, 9 aa, Artificial Sequence
  CDP Peptide
FLPTPVLED

SEQ ID NO: 7 - protein, 5 aa, Artificial Sequence
  CDP Peptide
LLTPP

SEQ ID NO: 8 - protein, 7 aa, Artificial Sequence
  CDP Peptide
LLTPILA

SEQ ID NO: 9 - protein, 8 aa, Artificial Sequence
  CDP Peptide
PVTPPMSP

SEQ ID NO: 10 - protein, 7 aa, Artificial Sequence
  CDP Peptide
ILTPPTT

SEQ ID NO: 11 - protein, 7 aa, Artificial Sequence
  CDP Peptide
LITPPTT

SEQ ID NO: 12 - protein, 19 aa, Artificial Sequence
  CDP Peptide
ASPLPSGLLTPPQSGKKQS

SEQ ID NO: 13 - protein, 14 aa, Artificial Sequence
  CDP Peptide
APPLSQETFSDLWK

SEQ ID NO: 14 - protein, 5 aa, Artificial Sequence
  CDP Peptide
LLTPP

SEQ ID NO: 15 - protein, 9 aa, Artificial Sequence
  CDP Peptide
GLLSPPQSG

SEQ ID NO: 16 - protein, 9 aa, Artificial Sequence
  CDP Peptide
GLLYPPQSG

SEQ ID NO: 17 - protein, 9 aa, Artificial Sequence
  CDP Peptide
GLLTPPQSG

SEQ ID NO: 18 - protein, 9 aa, Artificial Sequence
  CDP Peptide
GKLTPPQSG

SEQ ID NO: 19 - protein, 9 aa, Artificial Sequence
  CDP Peptide
GLKTPPQSG

SEQ ID NO: 20 - protein, 9 aa, Artificial Sequence
  CDP Peptide
GLLTAPQSG

SEQ ID NO: 21 - protein, 9 aa, Artificial Sequence
  CDP Peptide
GLLTPKQSG

SEQ ID NO: 22 - protein, 9 aa, Artificial Sequence
  CDP Peptide
GLLTPPKSG

SEQ ID NO: 23 - protein, 9 aa, Artificial Sequence
  CDP Peptide
GLLTPPQKG

SEQ ID NO: 24 - protein, 9 aa, Artificial Sequence
  CDP Peptide
GLLTPPQSK

SEQ ID NO: 25 - protein, 9 aa, Artificial Sequence
  CDP Peptide
GLLTPPKSG

SEQ ID NO: 26 - protein, 9 aa, Artificial Sequence
  CDP Peptide
FLPTPVLED

SEQ ID NO: 27 - protein, 20 aa, Artificial Sequence
  CDP Peptide
PKPLNLSKPISPPPSLKKTA

SEQ ID NO: 28 - protein, 9 aa, Artificial Sequence
  CDP Peptide
PPVTPPMSP

SEQ ID NO: 29 - protein, 9 aa, Artificial Sequence
  CDP Peptide
VPVTPSTTK

SEQ ID NO: 30 - protein, 14 aa, Artificial Sequence
  CDP Peptide
TGEFPQFTPQEQLI

SEQ ID NO: 31 - protein, 9 aa, Artificial Sequence
  CDP Peptide
VEQTPKKPG

SEQ ID NO: 32 - protein, 11 aa, Artificial Sequence
  CDP Peptide
TSFLPTPVLED

SEQ ID NO: 33 - protein, 4 aa, Artificial Sequence
  CDP Peptide
LPTP

SEQ ID NO: 34 - protein, 4 aa, Artificial Sequence
  CDP Peptide
GPTP

SEQ ID NO: 35 - protein, 4 aa, Artificial Sequence
  CDP Peptide
YPTP

SEQ ID NO: 36 - protein, 12 aa, Artificial Sequence
  TGF - beta peptide
CMHIESLDSYTC

SEQ ID NO: 37 - protein, 12 aa, Artificial Sequence
  TGF - beta peptide
CMYIEALDKYAC

SEQ ID NO: 38 - protein, 32 aa, Artificial Sequence
  PH-dependent internalizing peptide
XXXEAALAEALAEALAEALAEALAEALEALAA

SEQ ID NO: 39 - protein, 8 aa, Artificial Sequence
  substrate for N-myristoyl transferase
GNAAAARR

SEQ ID NO: 40 - protein, 10 aa, Artificial Sequence
  laminin peptide
CDPGYIGSRC

SEQ ID NO: 41 - DNA, 75 bp, Artificial Sequence
  RGD/SV40
catatgggtggctgccgtggcgatatgttcggttgcggtgctcctccaaaaaagaagagaaaggtagctggattc

SEQ ID NO: 42 - protein, 24 aa, Artificial Sequence
  RGD/SV40
MGGCRGDMFGCGAPPKKKRKVAGF

SEQ ID NO: 43 - protein, 72 aa, Artificial Sequence
  HIV-1 tat (1-72)
MEPVDPRLEPWKHPGSQPKTACTNCYCKKCCFHCQVCFITKALGISYGRKKRRQRRRPPQGSQTHQVSLSKQ

SEQ ID NO: 44 - DNA, 912 bp, Artificial Sequence
  HIV-1 VP22
catatgacctctcgccgctccgtgaagtcgggtccgcgggaggttccgcgcgatgagtacgaggatctgtactacaccccgtcttcaggtatggcgagtcccgatagtccgcctgacacctcccgccgtggcgccctacagacacgctcgcgccagaggggcgaggtccgtttcgtccagtacgacgagtcggattatgccctctacgggggctcgtcatccgaagacgacgaacacccggaggtcccccggacgcggcgtcccgtttccggggcggttttgtccggcccggggcctgcgcgggcgcctccgccacccgctgggtccggaggggccggacgcacacccaccaccgccccccgggccccccgaacccagcgggtggcgactaaggcccccgcggccccggcggcggagaccacccgcggcaggaaatcggcccagccagaatccgccgcactcccagacgcccccgcgtcgacggcgccaacccgatccaagacacccgcgcaggggctggccagaaagctgcactttagcaccgcccccccaaaccccgacgcgccatggaccccccgggtggccggctttaacaagcgcgtcttctgcgccgcggtcgggcgcctggcggccatgcatgcccggatggcggcggtccagctctgggacatgtcgcgtccgcgcacagacgaagacctcaacgaactccttggcatcaccaccatccgcgtgacggtctgcgagggcaaaaacctgcttcagcgcgccaacgagttggtgaatccagacgtggtgcaggacgtcgacgcggccacggcgactcgagggcgttctgcggcgtcgcgccccaccgagcgacctcgagccccagcccgctccgcttctcgccccagacggcccgtcgaggaattc

SEQ ID NO: 45 - protein, 301 aa, Artificial Sequence
  HIV-1 VP22
MTSRRSVKSGPREVPRDEYEDLYYTPSSGMASPDSPPDTSRRGALQTRSRQRGEVRFVQYDESDYALYGGSSSEDDEHPEVPRTRRPVSGAVLSGPGPARAPPPPAGSGGAGRTPTTAPRAPRTGRVATKAPAAPAAETTRGRKSAQPESAALPDAPASTAPTRSKTPAQGLARKLHFSTAPPNPDAPWTPRVAGFNKRVFCAAVGRLAAMHARMAAVQLWDMSRPRTDEDLNELLGITTIRVTVCEGKNLLQRANELVNPDVVQDVDAATATRGRSAASRPTERPRAPARSASRPRRPVE

SEQ ID NO: 46 - DNA, 120 bp, Artificial Sequence
  c-terminus of VP22
catatggacgtcgacgcggccacggcgactcgagggcgttctgcggcgtcgcgccccaccgagcgacctcgagccccagcccgctccgcttctcgccccagacggcccgtcgaggaattc

SEQ ID NO: 47 - protein, 37 aa, Artificial Sequence
  c-terminus of VP22
MDVDAATATRGRSAASRPTERPRAPARSASRPRRPVE

SEQ ID NO: 48 - protein, 5 aa, Artificial Sequence
  CycE-5mer
LLTPP

SEQ ID NO: 49 - protein, 9 aa, Artificial Sequence
  CycEpS-9mer
GLLSPPQSG

SEQ ID NO: 50 - protein, 9 aa, Artificial Sequence
  CycEpY-9mer
GLLYPPQSG

SEQ ID NO: 51 - protein, 9 aa, Artificial Sequence
  CycEdeP-9mer
GLLTPPQSG

SEQ ID NO: 52 - protein, 9 aa, Artificial Sequence
  CycEK-2-9mer
GKLTPPQSG

SEQ ID NO: 53 - protein, 9 aa, Artificial Sequence
  CycEK-1-9mer
GLKTPPQSG

SEQ ID NO: 54 - protein, 9 aa, Artificial Sequence
  CycEA1-9mer
GLLTAPQSG

SEQ ID NO: 55 - protein, 9 aa, Artificial Sequence
  CycEK2-9mer
GLLTPKQSG

SEQ ID NO: 56 - protein, 9 aa, Artificial Sequence
  CycEK3-9mer
GLLTPPKSG

SEQ ID NO: 57 - protein, 9 aa, Artificial Sequence
  CycEK3Ac-9mer
GLLTPPKSG

SEQ ID NO: 58 - protein, 9 aa, Artificial Sequence
  CycEK4-9mer
GLLTPPQKG

SEQ ID NO: 59 - protein, 9 aa, Artificial Sequence
  CycEK5-9mer
GLLTPPQSK

SEQ ID NO: 60 - protein, 20 aa, Artificial Sequence
  Far1pS87
PKPLNLSKPISPPPSLKKTA

SEQ ID NO: 61 - protein, 9 aa, Artificial Sequence
  Ash1pT290
PPVTPPMSP

SEQ ID NO: 62 - protein, 9 aa, Artificial Sequence
  SicpT45
VPVTPSTTK

SEQ ID NO: 63 - protein, 14 aa, Artificial Sequence
  FarpT306
TGEFPQFTPQEQLI

SEQ ID NO: 64 - protein, 9 aa, Artificial Sequence
  p27pT187
VEQTPKKPG

SEQ ID NO: 65 - protein, 13 aa, Artificial Sequence
  Cdc16
LSKNLLTPQEEWD

SEQ ID NO: 66 - protein, 9 aa, Artificial Sequence
  Pcl7
ELLTPILAF

SEQ ID NO: 67 - protein, 9 aa, Artificial Sequence
  Pcl2
NVQTPTLMA

SEQ ID NO: 68 - DNA, 40 bp, Artificial Sequence
  T2A/T5A
ccggatccatatggctcctagcgcgccaccaaggtccaga

SEQ ID NO: 69 - DNA, 30 bp, Artificial Sequence
  T33A
atgcaaggtcaaaaggcgccccaaaagcct

SEQ ID NO: 70 - DNA, 63 bp, Artificial Sequence
  S69A/S80A
atgggtatgaccgctccatttaatgggcttacgtctcctcaacgggccccgtttccaaaatct

SEQ ID NO: 71 - DNA, 32 bp, Artificial Sequence
  T173A
ttaaagatgtacctggcgcccccagcgacaag

SEQ ID NO: 72 - DNA, 30 bp, Artificial Sequence
  S191V
aattggaacaacaacgttccgaaaaatgac

SEQ ID NO: 73 - DNA, 32 bp, Artificial Sequence
  T45A
cagaacctagtcccgctagctccctcaacaac

SEQ ID NO: 74 - DNA, 63 bp, Artificial Sequence
  S69A/S76A/S80A
atgggtatgaccgctccatttaatgggcttacggctcctcaacgggccccgtttccaaaatct

SEQ ID NO: 75 - DNA, 88 bp, Artificial Sequence
  T45::cycE19
cacagaacctagtcgctagccctctcccctcaggcctcctcaccccgccacagagcggtaagaagcagagcaagtcttttaaaaatgc

SEQ ID NO: 76 - DNA, 63 bp, Artificial Sequence
  S69A/S76LLTPP/S80A
atgggtatgaccgctccatttaatgggcttctgactcctccaggggccccgtttccaaaatct

SEQ ID NO: 77 - DNA, 51 bp, Artificial Sequence
  T45LLTPP
cagaacctagtccttctcactcccccaacaaccggttcctttaaaaatgcg

SEQ ID NO: 78 - DNA, 24 bp, Artificial Sequence
  T45PSR
gtcactccctcgagaactaagtct